SEQUENCE LISTING (275 sequences)

SEQ ID NO: 1 - DNA, 473 bp, synthetic construct
  source 1..473: mol_type = other DNA
tcccggggtttccgccattttttggtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggaaacctgatcatgtagatcgaacggactctaaatccgttcagccgggttagattcccggggtttccgccattttttcctagacccagctttcttgtacaaagttgg

SEQ ID NO: 2 - DNA, 473 bp, synthetic construct
  source 1..473: mol_type = other DNA
tcccggggtttccgccattttttggtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggaaacctgatcatgtagatcgaacggactttaaatccgttcagccgggttagattcccggggtttccgccattttttcctagacccagctttcttgtacaaagttgg

SEQ ID NO: 3 - DNA, 473 bp, synthetic construct
  source 1..473: mol_type = other DNA
tcccggggtttccgccattttttggtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggaaacctgatcatgtagatcgaacggacttcaaatccgttcagccgggttagattcccggggtttccgccattttttcctagacccagctttcttgtacaaagttgg

SEQ ID NO: 4 - DNA, 1449 bp, synthetic construct
  source 1..1449: mol_type = other DNA
cagcctccggactctagaggatcgaacccttaaggccaccatggataagaaacctttgaacactctcattagtgcgacagggctctggatgtcccgaacggggactatacacaagataaaacaccatgaggtctcaaggagcaaaatctatatcgagatggcatgcggcgaccatcttgtggtaaataatagtaggtcctccaggacggcaagagcactccgacatcacaagtacagaaaaacctgcaaacggtgtagggtatccgacgaagacttgaacaaatttttgactaaggccaacgaggatcaaacttctgtcaaagtgaaagtggtttctgctcctacccgaactaagaaggccatgcccaagtccgtggcaagggcacccaagccactcgaaaatactgaggccgctcaggcccaaccatccggtagtaagttcagtccagccatacccgtaagtacccaagaatctgtcagtgtgccggcctcagtttccacatctataagttcaatttctacaggagcgacggcctccgccctcgtcaagggtaacacaaacccgataacttctatgagtgcccccgtacaggcatccgcaccagcactgacgaagtctcaaactgataggctggaagtgctcttgaatccgaaggacgagatatctcttaactccggtaaacctttccgggagctggaaagtgaacttctcagccggcgaaaaaaagacctccagcaaatttacgcagaggaaagggagaactatctggggaagttggaacgagagatcacccgattctttgtcgatcgcggatttttggagattaaaagcccaattctcatcccccttgaatatatcgaacgaatgggaatcgacaatgatacggagttgtccaagcagattttccgcgtagacaagaacttttgtcttcgacccatgctcgctccgaacctctacaattacttgagaaagttggacagagcgctcccggacccgatcaagatatttgagatcggtccttgttatagaaaggagagtgatggaaaagaacacctcgaagagttcacgatgctgaacttctgccaaatgggttctggctgcacacgggagaatctcgaaagcatcattacagatttccttaaccatctggggatagactttaaaatagtgggtgacagctgtatggtatacggagataccttggacgtaatgcacggggatcttgagctttcctccgccgtggttggacctataccgttggaccgggagtggggaatcgacaaaccgtggataggcgccggtttcggccttgaaagactcctcaaagtcaagcatgatttcaaaaacataaaacgggctgctcgctccgaatcttattacaacggtataagtacgaacctgtgataatagcttaagggttcgatccctactggttagtaatgagttta

SEQ ID NO: 5 - DNA, 72 bp, synthetic construct
  source 1..72: mol_type = other DNA
ggaaacctgatcatgtagatcgaatggactctaaatccgttcagccgggttagattcccggggtttccgcca

SEQ ID NO: 6 - DNA, 72 bp, synthetic construct
  source 1..72: mol_type = other DNA
ggggggtggatcgaatagatcacacggactctaaattcgtgcaggcgggtgaaactcccgtactccccgcca

SEQ ID NO: 7 - DNA, 72 bp, synthetic construct
  source 1..72: mol_type = other DNA
ggaaacctgatcatgtagatcgaatggactttaaatccgttcagccgggttagattcccggggtttccgcca

SEQ ID NO: 8 - DNA, 72 bp, synthetic construct
  source 1..72: mol_type = other DNA
ggaaacctgatcatgtagatcgaatggacttcaaatccgttcagccgggttagattcccggggtttccgcca

SEQ ID NO: 9 - DNA, 1260 bp, synthetic construct
  source 1..1260: mol_type = other DNA
atggataaaaaaccattagatgttttaatatctgcgaccgggctctggatgtccaggactggcacgctccacaaaatcaagcaccatgaggtctcaagaagtaaaatatacattgaaatggcgtgtggagaccatcttgttgtgaataattccaggagttgtagaacagccagagcattcagacatcataagtacagaaaaacctgcaaacgatgtagggtttcggacgaggatatcaataattttctcacaagatcaaccgaaagcaaaaacagtgtgaaagttagggtagtttctgctccaaaggtcaaaaaagctatgccgaaatcagtttcaagggctccgaagcctctggaaaattctgtttctgcaaaggcatcaacgaacacatccagatctgtaccttcgcctgcaaaatcaactccaaattcgtctgttcccgcatcggctcctgctccttcacttacaagaagccagcttgatagggttgaggctctcttaagtccagaggataaaatttctctaaatatggcaaagcctttcagggaacttgagcctgaacttgtgacaagaagaaaaaacgattttcagcggctctataccaatgatagagaagactacctcggtaaactcgaacgtgatattacgaaatttttcgtagaccggggttttctggagataaagtctcctatccttattccggcggaatacgtggagagaatgggtattaataatgatactgaactttcaaaacagatcttccgggtggataaaaatctctgcttgaggccaatgcttgccccgactctttacaactatctgcgaaaactcgataggattttaccaggcccaataaaaattttcgaagtcggaccttgttaccggaaagagtctgacggcaaagagcacctggaagaatttactatggtgaacttctgtcagatgggttcgggatgtactcgggaaaatcttgaagctctcatcaaagagtttctggactatctggaaatcgacttcgaaatcgtaggagattcctgtatggtctttggggatactcttgatataatgcacggggacctggagctttcttcggcagtcgtcgggccagtttctcttgatagagaatggggtattgacaaaccatggataggtgcaggttttggtcttgaacgcttgctcaaggttatgcacggctttaaaaacattaagagggcatcaaggtccgaatcttactataatgggatttcaaccaatctgtaa

SEQ ID NO: 10 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 11 - DNA, 723 bp, synthetic construct
  source 1..723: mol_type = other DNA
atggtgagcaagggcgaggagctgttcaccggggtggtgcccatcctggtcgagctggacggcgacgtaaacggccacaagttcagcgtgtccggcgagggcgagggcgatgccacctagggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatga

SEQ ID NO: 12 - DNA, 723 bp, synthetic construct
  source 1..723: mol_type = other DNA
atggtgagcaagggcgaggagctgttcaccggggtggtgcccatcctggtcgagctggacggcgacgtaaacggccacaagttcagcgtgtccggcgagggcgagggcgatgccacctaaggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatga

SEQ ID NO: 13 - DNA, 723 bp, synthetic construct
  source 1..723: mol_type = other DNA
atggtgagcaagggcgaggagctgttcaccggggtggtgcccatcctggtcgagctggacggcgacgtaaacggccacaagttcagcgtgtccggcgagggcgagggcgatgccacctgaggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatga

SEQ ID NO: 14 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 15 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 16 - DNA, 78 bp, synthetic construct
  source 1..78: mol_type = other DNA
ggaaacctgatcatgtagatcgaacggactctaaatccgttcagccgggttagattcccggggtttccgccatttttt

SEQ ID NO: 17 - DNA, 78 bp, synthetic construct
  source 1..78: mol_type = other DNA
ggaaacctgatcatgtagatcgaacggactttaaatccgttcagccgggttagattcccggggtttccgccatttttt

SEQ ID NO: 18 - DNA, 78 bp, synthetic construct
  source 1..78: mol_type = other DNA
ggaaacctgatcatgtagatcgaacggacttcaaatccgttcagccgggttagattcccggggtttccgccatttttt

SEQ ID NO: 19 - DNA, 48 bp, synthetic construct
  source 1..48: mol_type = other DNA
tcccggggtttccgccattttttggtactgagtcgcccagtctcagat

SEQ ID NO: 20 - DNA, 27 bp, synthetic construct
  source 1..27: mol_type = other DNA
caaacaaggcttttctccaagggatat

SEQ ID NO: 21 - DNA, 71 bp, synthetic construct
  source 1..71: mol_type = other DNA
ccttggagaaaagccttgtttgggaaacctgatcatgtagatcgaacggactctaaatccgttcagccggg

SEQ ID NO: 22 - DNA, 72 bp, synthetic construct; identical to SEQ ID NO: 5
  source 1..72: mol_type = other DNA

SEQ ID NO: 23 - DNA, 72 bp, synthetic construct
  source 1..72: mol_type = other DNA
ggaaacctgatcatgtagatcgaacggactctaaatccgttcagccgggttagattcccggggtttccgcca

SEQ ID NO: 24 - DNA, 73 bp, synthetic construct
  source 1..73: mol_type = other DNA
ggccgcgtggcctaatggataaggcgtctgacttcagatcagaagattgcaggttcgagtcctgccgcggtcg

SEQ ID NO: 25 - DNA, 73 bp, synthetic construct
  source 1..73: mol_type = other DNA
gaccacgtggcctaatggataaggcgtctgacttcagatcagaagattgagggttcgaatcccttcgtggtta

SEQ ID NO: 26 - DNA, 82 bp, synthetic construct
  source 1..82: mol_type = other DNA
gtagtcgtggccgagtggttaaggcgatggactctaaatccattggggtttccccgcgcaggttcgaatcctgccgactacg

SEQ ID NO: 27 - DNA, 83 bp, synthetic construct
  source 1..83: mol_type = other DNA
gtcaggatggccgagtggtctaaggcgccagactctagttctggtctccaatggaggcgtgggttcgaatcccacttctgaca

SEQ ID NO: 28 - DNA, 21 bp, synthetic construct
  source 1..21: mol_type = other DNA
ttgtggaaaggacgaaacacc

SEQ ID NO: 29 - DNA, 31 bp, synthetic construct
  source 1..31: mol_type = other DNA
acaagaaagctgggtctaggctagcaaaaaa

SEQ ID NO: 30 - DNA, 76 bp, synthetic construct
  source 1..76: mol_type = other DNA
ttgtggaaaggacgaaacaccggtcaggatggccgagtggtctaaggcgccagactctagttctggtctccaatgg

SEQ ID NO: 31 - DNA, 76 bp, synthetic construct
  source 1..76: mol_type = other DNA
ttgtggaaaggacgaaacaccggtcaggatggccgagtggtctaaggcgccagactttagttctggtctccaatgg

SEQ ID NO: 32 - DNA, 76 bp, synthetic construct
  source 1..76: mol_type = other DNA
ttgtggaaaggacgaaacaccggtcaggatggccgagtggtctaaggcgccagacttcagttctggtctccaatgg

SEQ ID NO: 33 - DNA, 77 bp, synthetic construct
  source 1..77: mol_type = other DNA
acaagaaagctgggtctaggctagcaaaaaatgtcagaagtgggattcgaacccacgcctccattggagaccagaac

SEQ ID NO: 34 - DNA, 75 bp, synthetic construct
  source 1..75: mol_type = other DNA
ttgtggaaaggacgaaacaccggtagtcgtggccgagtggttaaggcgatggactctaaatccattggggtttcc

SEQ ID NO: 35 - DNA, 75 bp, synthetic construct
  source 1..75: mol_type = other DNA
ttgtggaaaggacgaaacaccggtagtcgtggccgagtggttaaggcgatggactttaaatccattggggtttcc

SEQ ID NO: 36 - DNA, 75 bp, synthetic construct
  source 1..75: mol_type = other DNA
ttgtggaaaggacgaaacaccggtagtcgtggccgagtggttaaggcgatggacttcaaatccattggggtttcc

SEQ ID NO: 37 - DNA, 77 bp, synthetic construct
  source 1..77: mol_type = other DNA
acaagaaagctgggtctaggctagcaaaaaacgtagtcggcaggattcgaacctgcgcggggaaaccccaatggatt

SEQ ID NO: 38 - DNA, 77 bp, synthetic construct
  source 1..77: mol_type = other DNA
ttgtggaaaggacgaaacaccggaccacgtggcctaatggataaggcgtctgacttcagatcagaagattgagggtt

SEQ ID NO: 39 - DNA, 77 bp, synthetic construct
  source 1..77: mol_type = other DNA
ttgtggaaaggacgaaacaccggaccacgtggcctaatggataaggcgtctgactttagatcagaagattgagggtt

SEQ ID NO: 40 - DNA, 77 bp, synthetic construct; identical to SEQ ID NO: 38
  source 1..77: mol_type = other DNA

SEQ ID NO: 41 - DNA, 68 bp, synthetic construct
  source 1..68: mol_type = other DNA
acaagaaagctgggtctaggctagcaaaaaataaccacgaagggattcgaaccctcaatcttctgatc

SEQ ID NO: 42 - DNA, 429 bp, synthetic construct
  source 1..429: mol_type = other DNA
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggtagtcgtggccgagtggttaaggcgatggactttaaatccattggggtttccccgcgcaggttcgaatcctgccgactacgtttttt

SEQ ID NO: 43 - DNA, 41 bp, synthetic construct
  source 1..41: mol_type = other DNA
aatcctgccgactacgttttttgtactgagtcgcccagtct

SEQ ID NO: 44 - DNA, 19 bp, synthetic construct
  source 1..19: mol_type = other DNA
tttgaaagagcaataaaat

SEQ ID NO: 45 - DNA, 19 bp, synthetic construct
  source 1..19: mol_type = other DNA
ctttgaaagagcaatagaa

SEQ ID NO: 46 - DNA, 19 bp, synthetic construct
  source 1..19: mol_type = other DNA
tttgaaagagcaataaaat

SEQ ID NO: 47 - DNA, 19 bp, synthetic construct
  source 1..19: mol_type = other DNA
ataaaatggcttcaactat

SEQ ID NO: 48 - DNA, 19 bp, synthetic construct
  source 1..19: mol_type = other DNA
aatagaatggcttcaacta

SEQ ID NO: 49 - DNA, 19 bp, synthetic construct
  source 1..19: mol_type = other DNA
aataaaatggcttcaacta

SEQ ID NO: 50 - DNA, 22 bp, synthetic construct
  source 1..22: mol_type = other DNA
tcacagacaccgctcagtttgt

SEQ ID NO: 51 - DNA, 16 bp, synthetic construct
  source 1..16: mol_type = other DNA
atgccacctggggcaa

SEQ ID NO: 52 - DNA, 16 bp, synthetic construct
  source 1..16: mol_type = other DNA
tgccacctggggcaag

SEQ ID NO: 53 - DNA, 16 bp, synthetic construct
  source 1..16: mol_type = other DNA
gccacctggggcaagc

SEQ ID NO: 54 - DNA, 18 bp, synthetic construct
  source 1..18: mol_type = other DNA
gatgccacctggggcaag

SEQ ID NO: 55 - DNA, 20 bp, synthetic construct
  source 1..20: mol_type = other DNA
gcgatgccacctggggcaag

SEQ ID NO: 56 - DNA, 49 bp, synthetic construct
  source 1..49: mol_type = other DNA
gggtggaatagtataacaatatgctaaatgttgttatagtatcccacct

SEQ ID NO: 57 - DNA, 45 bp, synthetic construct
  source 1..45: mol_type = other DNA
gtggaatagtataacaatatgctaaatgttgttatagtatcccac

SEQ ID NO: 58 - DNA, 24 bp, synthetic construct
  source 1..24: mol_type = other DNA
gggccctcttcagggccctctaga

SEQ ID NO: 59 - DNA, 14 bp, synthetic construct
  source 1..14: mol_type = other DNA
atcgccctgaaaag

SEQ ID NO: 60 - DNA, 11 bp, synthetic construct
  source 1..11: mol_type = other DNA
gccacctgggg

SEQ ID NO: 61 - DNA, 82 bp, synthetic construct
  source 1..82: mol_type = other DNA
gtagtcgtggccgagtggttaaggcgatggactnnnaatccattggggtttccccgcgcaggttcgaatcctgccgactacg

SEQ ID NO: 62 - DNA, 83 bp, synthetic construct
  source 1..83: mol_type = other DNA
gtcaggatggccgagtggtctaaggcgccagactnnngttctggtctccaatggaggcgtgggttcgaatcccacttctgaca

SEQ ID NO: 63 - DNA, 73 bp, synthetic construct
  source 1..73: mol_type = other DNA
gaccacgtggcctaatggataaggcgtctgactnnngatcagaagattgagggttcgaatcccttcgtggtta

SEQ ID NO: 64 - DNA, 28 bp, synthetic construct
  source 1..28: mol_type = other DNA
gtgttactgaatatgaaataatggagga

SEQ ID NO: 65 - DNA, 24 bp, synthetic construct
  source 1..24: mol_type = other DNA
atttctggcatatttctgaaggtg

SEQ ID NO: 66 - DNA, 29 bp, synthetic construct
  source 1..29: mol_type = other DNA
ctctctgtaccttatcttagtgttactga

SEQ ID NO: 67 - DNA, 29 bp, synthetic construct
  source 1..29: mol_type = other DNA
ctcttcaaattctgacagatatttctggc

SEQ ID NO: 68 - DNA, 22 bp, synthetic construct
  source 1..22: mol_type = other DNA
acccttcctttcttaccacaca

SEQ ID NO: 69 - DNA, 23 bp, synthetic construct
  source 1..23: mol_type = other DNA
cagggtgtccagatctgattgtt

SEQ ID NO: 70 - DNA, 29 bp, synthetic construct
  source 1..29: mol_type = other DNA
cttctcttttaaactaacccatcagagtt

SEQ ID NO: 71 - DNA, 20 bp, synthetic construct
  source 1..20: mol_type = other DNA
tgtctgtggcgagccaaaca

SEQ ID NO: 72 - DNA, 20 bp, synthetic construct
  source 1..20: mol_type = other DNA
actttctcgttaccttaccg

SEQ ID NO: 73 - protein, 585 aa, synthetic construct
  REGION 584..585: note = MISC_FEATURE - May or may not be present
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSGPKKRKVAA

SEQ ID NO: 74 - protein, 560 aa, synthetic construct
  SITE 560: note = MISC_FEATURE - May or may not be present
MGPKKKRKVAAGSGSGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGSGGSGGSMLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 75 - protein, 492 aa, synthetic construct
  REGION 491..492: note = MISC_FEATURE - May or may not be present
MGNARTRRRERRAEKQAQWKAANGGGGTSGSGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSGPKKRKVAA

SEQ ID NO: 76 - protein, 469 aa, synthetic construct
  REGION 1..2: note = MISC_FEATURE - May or may not be present
  SITE 469: note = MISC_FEATURE - May or may not be present
MGPKKKRKVAAGSGSGSMGNARTRRRERRAEKQAQWKAANGGGGTSGSGSGSPAGGGAPGSGGGSMLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 77 - protein, 585 aa, synthetic construct
  REGION 584..585: note = MISC_FEATURE - May or may not be present
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSGPKKRKVAA

SEQ ID NO: 78 - protein, 560 aa, synthetic construct
  REGION 1..2: note = MISC_FEATURE - May or may not be present
  SITE 560: note = MISC_FEATURE - May or may not be present
MGPKKKRKVAAGSGSGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGSGGSGGSMLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 79 - protein, 492 aa, synthetic construct
  REGION 491..492: note = MISC_FEATURE - May or may not be present
MGNARTRRRERRAEKQAQWKAANGGGGTSGSGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSGPKKRKVAA

SEQ ID NO: 80 - protein, 469 aa, synthetic construct
  REGION 1..2: note = MISC_FEATURE - May or may not be present
  SITE 469: note = MISC_FEATURE - May or may not be present
MGPKKKRKVAAGSGSGSMGNARTRRRERRAEKQAQWKAANGGGGTSGSGSGSPAGGGAPGSGGGSMLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 81 - protein, 392 aa, synthetic construct
  REGION 1..2: note = MISC_FEATURE - May or may not be present
MGPKKKRKVAAGSGSGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGSGGSGGSMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 82 - protein, 385 aa, synthetic construct
  REGION 1..2: note = MISC_FEATURE - May or may not be present
MGPKKKRKVAAGSGSGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGSGGSGGSMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 83 - protein, 536 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNGGSGGSGGSMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 84 - protein, 543 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNGGSGGSGGSMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 85 - protein, 585 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSLPPLERLTL

SEQ ID NO: 86 - protein, 539 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSGSPKKKRKV

SEQ ID NO: 87 - protein, 541 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSGSLPPLERLTL

SEQ ID NO: 88 - protein, 399 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSSGSETPGTSESATPESMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKGSGSGSPKKKRKV

SEQ ID NO: 89 - protein, 401 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSSGSETPGTSESATPESMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKGSGSGSLPPLERLTL

SEQ ID NO: 90 - protein, 406 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSSGSETPGTSESATPESMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWRGSGSGSPKKKRKV

SEQ ID NO: 91 - protein, 408 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSSGSETPGTSESATPESMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWRGSGSGSLPPLERLTL

SEQ ID NO: 92 - protein, 9 aa, synthetic construct
GGSGGSGGS

SEQ ID NO: 93 - protein, 16 aa, synthetic construct
SGSETPGTSESATPES

SEQ ID NO: 94 - protein, 569 aa, synthetic construct
MPKKKRKVDPKKKRKVDPKKKRKVGSYPYDVPDYAGSNARTRRRERRAEKQAQWKAANGGGGSGGGGSGGGGSNARTRRRERRAEKQAQWKAANGGGGSGGGGSGGGGSNARTRRRERRAEKQAQWKAANGGGGSGGGGSGGGGSNARTRRRERRAEKQAQWKAANLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 95 - protein, 294 aa, synthetic construct
MPKKKRKVDGSGNARTRRRERRAEKQAQWKAANGGGGTSGSGSGSPAGGGAPGSGGGSMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 96 - protein, 402 aa, synthetic construct
MPKKKRKVDPKKKRKVDPKKKRKVGSYPYDVPDYAGSNARTRRRERRAEKQAQWKAANGGGGSGGGGSGGGGSNARTRRRERRAEKQAQWKAANGGGGSGGGGSGGGGSNARTRRRERRAEKQAQWKAANGGGGSGGGGSGGGGSNARTRRRERRAEKQAQWKAANMTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 97 - protein, 564 aa, synthetic construct
MLRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 98 - DNA, 54 bp, synthetic construct
  source 1..54: mol_type = other DNA
nnnnnnnnnnnnnnnnnnnnggccaacatgaggatcacccatgtctgcagggcc

SEQ ID NO: 99 - DNA, 62 bp, synthetic construct
  source 1..62: mol_type = other DNA
aacatgaggatcacccatgtcnnnnnnnnnnnnnnnnnnnnaacatgaggatcacccatgtc

SEQ ID NO: 100 - DNA, 39 bp, synthetic construct
  source 1..39: mol_type = other DNA
nnnnnnnnnnnnnnnnnnnngggccctgaagaagggccc

SEQ ID NO: 101 - DNA, 58 bp, synthetic construct
  source 1..58: mol_type = other DNA
gggccctgaagaagggcccnnnnnnnnnnnnnnnnnnnngggccctgaagaagggccc

SEQ ID NO: 102 - DNA, 49 bp, synthetic construct
  source 1..49: mol_type = other DNA
nnnnnnnnnnnnnnnnnnnnccggagcagacgatatggcgtcgctccgg

SEQ ID NO: 103 - DNA, 109 bp, synthetic construct
  source 1..109: mol_type = other DNA
tggaatagtataacaatatgctaaatgttgttatagtatcccacnnnnnnnnnnnnnnnnnnnngtggaatagtataacaatatgctaaatgttgttatagtatcccac

SEQ ID NO: 104 - DNA, 68 bp, synthetic construct
  source 1..68: mol_type = other DNA
gggtggaatagtataacaatatgctaaatgttgttatagtatcccacctnnncnnnnnnnnnnnnnnn

SEQ ID NO: 105 - DNA, 65 bp, synthetic construct
  source 1..65: mol_type = other DNA
gtggaatagtataacaatatgctaaatgttgttatagtatcccacnnnnnncnnnnnnnnnnnnn

SEQ ID NO: 106 - DNA, 66 bp, synthetic construct
  source 1..66: mol_type = other DNA
gtggaagaggagaacaatatgctaaatgttgttctcgtctcccacnnnnnncnnnnnnnnnnnnnn

SEQ ID NO: 107 - DNA, 68 bp, synthetic construct
  source 1..68: mol_type = other DNA
gggtggaagaggagaacaatatgctaaatgttgttctcgtctcccacctnnncnnnnnnnnnnnnnnn

SEQ ID NO: 108 - DNA, 66 bp, synthetic construct
  source 1..66: mol_type = other DNA
ggtgaagaggagaacaatatgctaaatgttgttctcgtctccaccnnnnnncnnnnnnnnnnnnnn

SEQ ID NO: 109 - DNA, 66 bp, synthetic construct
  source 1..66: mol_type = other DNA
ggtgaagaggagaacaatatgctaaatgttgttctcgtctccaccnnnnnnncnnnnnnnnnnnnn

SEQ ID NO: 110 - DNA, 66 bp, synthetic construct
  source 1..66: mol_type = other DNA
gtggaagaggagaacaataggctaaacgttgttctcgtctcccacnnnnnncnnnnnnnnnnnnnn

SEQ ID NO: 111 - DNA, 68 bp, synthetic construct
  source 1..68: mol_type = other DNA
gggtggaagaggagaacaataggctaaacgttgttctcgtctcccacctnnncnnnnnnnnnnnnnnn

SEQ ID NO: 112 - DNA, 66 bp, synthetic construct
  source 1..66: mol_type = other DNA
ggtgaagaggagaacaataggctaaacgttgttctcgtctccaccnnnnnncnnnnnnnnnnnnnn

SEQ ID NO: 113 - DNA, 66 bp, synthetic construct
  source 1..66: mol_type = other DNA
ggtgaagaggagaacaataggctaaacgttgttctcgtctccaccnnnnnnncnnnnnnnnnnnnn

SEQ ID NO: 114 - DNA, 73 bp, synthetic construct
  source 1..73: mol_type = other DNA
ggtgtcgagaatagtataacaatatgctaaatgttgttatagtatcctcgacaccnnnnnnncnnnnnnnnnn

SEQ ID NO: 115 - DNA, 73 bp, synthetic construct
  source 1..73: mol_type = other DNA
ggtgtcgagaagaggagaacaatatgctaaatgttgttctcgtctcctcgacaccnnnnnnncnnnnnnnnnn

SEQ ID NO: 116 - DNA, 73 bp, synthetic construct
  source 1..73: mol_type = other DNA
ggtgtcgagaagaggagaacaataggctaaacgttgttctcgtctcctcgacaccnnnnnnncnnnnnnnnnn

SEQ ID NO: 117 - protein, 1387 aa, synthetic construct
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGLPPLERLTLGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 118 - DNA, 65 bp, synthetic construct
  misc_feature 1..65: note = Description of Artificial Sequence: Syntheticoligonucleotide
  source 1..65: mol_type = other DNA
gtggaatagtataacaatatgctaaatgttgttatagtatcccacacaaaccgagcggtgtctgt

SEQ ID NO: 119 - DNA, 110 bp, synthetic construct
  source 1..110: mol_type = other DNA
gtggaatagtataacaatatgctaaatgttgttatagtatcccaccaaaccgagcggtgtctgtggtggaatagtataacaatatgctaaatgttgttatagtatcccac

SEQ ID NO: 120 - DNA, 110 bp, synthetic construct
  source 1..110: mol_type = other DNA
gtggaatagtataacaatatgctaaatgttgttatagtatcccactacaaaccgagcggtgtctggtggaatagtataacaatatgctaaatgttgttatagtatcccac

SEQ ID NO: 121 - DNA, 110 bp, synthetic construct
  source 1..110: mol_type = other DNA
gtggaatagtataacaatatgctaaatgttgttatagtatcccactttacaaaccgagcggtgtcgtggaatagtataacaatatgctaaatgttgttatagtatcccac

SEQ ID NO: 122 - DNA, 110 bp, synthetic construct
  source 1..110: mol_type = other DNA
gtggaatagtataacaatatgctaaatgttgttatagtatcccacgttttacaaaccgagcggtggtggaatagtataacaatatgctaaatgttgttatagtatcccac

SEQ ID NO: 123 - DNA, 110 bp, synthetic construct
  source 1..110: mol_type = other DNA
gtggaatagtataacaatatgctaaatgttgttatagtatcccacaagttttacaaaccgagcgggtggaatagtataacaatatgctaaatgttgttatagtatcccac

SEQ ID NO: 124 - protein, 429 aa, unidentified
  REGION 1..429: note = Description of Unknown:ADAR1 catalytic domain sequence
KAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 125 - protein, 385 aa, unidentified
  REGION 1..385: note = Description of Unknown:ADAR2 catalytic domain sequence
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 126 - protein, 298 aa, unidentified
  REGION 1..298: note = Description of Unknown:ADAR dsRBD sequence
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFN

SEQ ID NO: 127 - protein, 564 aa, synthetic construct; identical to SEQ ID NO: 97

SEQ ID NO: 128 - DNA, 36 bp, Mus sp.
  source 1..36: mol_type = other DNA
ctcacagacaccgctcggtttgtaaaacttttcttc

SEQ ID NO: 129 - DNA, 36 bp, synthetic construct
  source 1..36: mol_type = other DNA
ctcacagacaccgctcagtttgtaaaacttttcttc

SEQ ID NO: 130 - DNA, 36 bp, synthetic construct; identical to SEQ ID NO: 129
  source 1..36: mol_type = other DNA

SEQ ID NO: 131 - DNA, 36 bp, synthetic construct
  source 1..36: mol_type = other DNA
ctcacagacaccgctcatgtcttatctagcatgaca

SEQ ID NO: 132 - DNA, 36 bp, synthetic construct
  source 1..36: mol_type = other DNA
ctcacagacaccgctcgtgtcttatctagcatgaca

SEQ ID NO: 133 - DNA, 68 bp, synthetic construct
  source 1..68: mol_type = other DNA
nnnnnnnnnnnnnnncnnntccaccctatgatattgttgtaaatcgtataacaatatgataaggtggg

SEQ ID NO: 134 - DNA, 65 bp, synthetic construct
  source 1..65: mol_type = other DNA
nnnnnnnnnnnnncnnnnnncaccctatgatattgttgtaaatcgtataacaatatgataaggtg

SEQ ID NO: 135 - DNA, 66 bp, synthetic construct
  source 1..66: mol_type = other DNA
nnnnnnnnnnnnnncnnnnnncaccctctgctcttgttgtaaatcgtataacaagaggagaaggtg

SEQ ID NO: 136 - DNA, 68 bp, synthetic construct
  source 1..68: mol_type = other DNA
nnnnnnnnnnnnnnncnnntccaccctctgctcttgttgtaaatcgtataacaagaggagaaggtggg

SEQ ID NO: 137 - DNA, 66 bp, synthetic construct
  source 1..66: mol_type = other DNA
nnnnnnnnnnnnnncnnnnnnccacctctgctcttgttgtaaatcgtataacaagaggagaagtgg

SEQ ID NO: 138 - DNA, 66 bp, synthetic construct
  source 1..66: mol_type = other DNA
nnnnnnnnnnnnncnnnnnnnccacctctgctcttgttgtaaatcgtataacaagaggagaagtgg

SEQ ID NO: 139 - DNA, 66 bp, synthetic construct
  source 1..66: mol_type = other DNA
nnnnnnnnnnnnnncnnnnnncaccctctgctcttgttgcaaatcggataacaagaggagaaggtg

SEQ ID NO: 140 - DNA, 68 bp, synthetic construct
  source 1..68: mol_type = other DNA
nnnnnnnnnnnnnnncnnntccaccctctgctcttgttgcaaatcggataacaagaggagaaggtggg

SEQ ID NO: 141 - DNA, 66 bp, synthetic construct
  source 1..66: mol_type = other DNA
nnnnnnnnnnnnnncnnnnnnccacctctgctcttgttgcaaatcggataacaagaggagaagtgg

SEQ ID NO: 142 - DNA, 66 bp, synthetic construct
  source 1..66: mol_type = other DNA
nnnnnnnnnnnnncnnnnnnnccacctctgctcttgttgcaaatcggataacaagaggagaagtgg

SEQ ID NO: 143 - DNA, 73 bp, synthetic construct
  source 1..73: mol_type = other DNA
nnnnnnnnnncnnnnnnnccacagctcctctgctcttgttgcaaatcggataacaagaggagaagagctgtgg

SEQ ID NO: 144 - DNA, 73 bp, synthetic construct
  source 1..73: mol_type = other DNA
nnnnnnnnnncnnnnnnnccacagctcctctgctcttgttgtaaatcgtataacaagaggagaagagctgtgg

SEQ ID NO: 145 - DNA, 73 bp, synthetic construct
  source 1..73: mol_type = other DNA
nnnnnnnnnncnnnnnnnccacagctcctatgatattgttgtaaatcgtataacaatatgataagagctgtgg

SEQ ID NO: 146 - DNA, 109 bp, synthetic construct; identical to SEQ ID NO: 103
  source 1..109: mol_type = other DNA

SEQ ID NO: 147 - DNA, 86 bp, synthetic construct
  source 1..86: mol_type = other DNA
caacattatcggggagttttgacctccaaggtgttgnnnnnnnnnnnnnnnnnnnnnnnnnnnnnnnnnnnnnnnnnnnnnnnnnn

SEQ ID NO: 148 - DNA, 102 bp, synthetic construct
  source 1..102: mol_type = other DNA
nnnnnnnnnnnnnnnnnnnnnngttttagtccctgaaaagggactaaaataaagagtttgcgggactctgcggggttacaatcccctaaaaccgcttttttt

SEQ ID NO: 149 - DNA, 41 bp, synthetic construct
  source 1..41: mol_type = other DNA
aaagtctcacagacaccgctcagtttgtaaaacttttcttc

SEQ ID NO: 150 - DNA, 65 bp, synthetic construct
  source 1..65: mol_type = other DNA
gtggaatagtataacaatatgctaaatgttgttatagtatcccactgtctgtggcgagccaaaca

SEQ ID NO: 151 - DNA, 66 bp, synthetic construct
  source 1..66: mol_type = other DNA
gtggaatagtataacaatatgctaaatgttgttatagtatcccacgtgtctgtggcgagccaaaca

SEQ ID NO: 152 - DNA, 65 bp, synthetic construct
  source 1..65: mol_type = other DNA
aagttttacaaaccgagcggcaccctatgatattgttgtaaatcgtataacaatatgataaggtg

SEQ ID NO: 153 - DNA, 109 bp, synthetic construct
  source 1..109: mol_type = other DNA
tggaatagtataacaatatgctaaatgttgttatagtatcccaccaaaccgagcggtgtctgtggtggaatagtataacaatatgctaaatgttgttatagtatcccac

SEQ ID NO: 154 - DNA, 54 bp, synthetic construct
  source 1..54: mol_type = other DNA
caaaccgagcggtgtctgtgggccaacatgaggatcacccatgtctgcagggcc

SEQ ID NO: 155 - DNA, 62 bp, synthetic construct
  source 1..62: mol_type = other DNA
aacatgaggatcacccatgtccaaaccgagcggtgtctgtgaacatgaggatcacccatgtc

SEQ ID NO: 156 - DNA, 39 bp, synthetic construct
  source 1..39: mol_type = other DNA
caaaccgagcggtgtctgtggggccctgaagaagggccc

SEQ ID NO: 157 - DNA, 58 bp, synthetic construct
  source 1..58: mol_type = other DNA
gggccctgaagaagggccccaaaccgagcggtgtctgtggggccctgaagaagggccc

SEQ ID NO: 158 - DNA, 16 bp, synthetic construct
  source 1..16: mol_type = other DNA
cggcctcagtgagcga

SEQ ID NO: 159 - DNA, 21 bp, synthetic construct
  source 1..21: mol_type = other DNA
ggaacccctagtgatggagtt

SEQ ID NO: 160 - DNA, 20 bp, synthetic construct
  misc_feature 1..20: note = Description of Unknown:target sequence
  source 1..20: mol_type = other DNA
gggaaatccagctagcggca

SEQ ID NO: 161 - DNA, 20 bp, synthetic construct
  misc_feature 1..20: note = Description of Unknown:target sequence
  source 1..20: mol_type = other DNA
gggaaaactgtctagttccc

SEQ ID NO: 162 - DNA, 20 bp, synthetic construct
  misc_feature 1..20: note = Description of Unknown:target sequence
  source 1..20: mol_type = other DNA
taattgactggctagtacag

SEQ ID NO: 163 - DNA, 20 bp, synthetic construct
  misc_feature 1..20: note = Description of Unknown:target sequence
  source 1..20: mol_type = other DNA
gagctttttgcttagcactg

SEQ ID NO: 164 - DNA, 1884 bp, synthetic construct
  source 1..1884: mol_type = other DNA
atggataaaaaaccattagatgttttaatatctgcgaccgggctctggatgtccaggactggcacgctccacaaaatcaagcaccatgaggtctcaagaagtaaaatatacattgaaatggcgtgtggagaccatcttgttgtgaataattccaggagttgtagaacagccagagcattcagacatcataagtacagaaaaacctgcaaacgatgtagggtttcggacgaggatatcaataattttctcacaagatcaaccgaaagcaaaaacagtgtgaaagttagggtagtttctgctccaaaggtcaaaaaagctatgccgaaatcagtttcaagggctccgaagcctctggaaaattctgtttctgcaaaggcatcaacgaacacatccagatctgtaccttcgcctgcaaaatcaactccaaattcgtctgttcccgcatcggctcctgctccttcacttacaagaagccagcttgatagggttgaggctctcttaagtccagaggataaaatttctctaaatatggcaaagcctttcagggaacttgagcctgaacttgtgacaagaagaaaaaacgattttcagcggctctataccaatgatagagaagactacctcggtaaactcgaacgtgatattacgaaatttttcgtagaccggggttttctggagataaagtctcctatccttattccggcggaatacgtggagagaatgggtattaataatgatactgaactttcaaaacagatcttccgggtggataaaaatctctgcttgaggccaatgcttgccccgactctttacaactatctgcgaaaactcgataggattttaccaggcccaataaaaattttcgaagtcggaccttgttaccggaaagagtctgacggcaaagagcacctggaagaatttactatggtgaacttctgtcagatgggttcgggatgtactcgggaaaatcttgaagctctcatcaaagagtttctggactatctggaaatcgacttcgaaatcgtaggagattcctgtatggtctttggggatactcttgatataatgcacggggacctggagctttcttcggcagtcgtcgggccagtttctcttgatagagaatggggtattgacaaaccatggataggtgcaggttttggtcttgaacgcttgctcaaggttatgcacggctttaaaaacattaagagggcatcaaggtccgaatcttactataatgggatttcaaccaatctgttataaagcggccgcgtcgacgggcccgcggaattccgccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggcca

SEQ ID NO: 165 - DNA, 2106 bp, synthetic construct
  source 1..2106: mol_type = other DNA
atggatatagaagatgaagaaaacatgagttccagcagcactgatctgaaggaaaaccgcaatctggacaacgtgtcccccaaggatggcagcacacctgggcctggcgagggctctcagctctccaatgggggtggtggtggccccggcagaaagcggcccctggaggagggcagcaatggccactccaagtaccgcctgaagaaaaggaggaaaacaccagggcccgtcctccccaagaacgccctgatgcagctgaatgagatcaagcctggtttgcagtacacactcctgtcccagactgggcccgtgcacgcgcctttgtttgtcatgtctgtggaggtgaatggccaggtttttgagggctctggtcccacaaagaaaaaggcaaaactccatgctgctgagaaggccttgaggtctttcgttcagtttcctaatgcctctgaggcccacctggccatggggaggaccctgtctgtcaacacggacttcacatctgaccaggccgacttccctgacacgctcttcaatggttttgaaactcctgacaaggcggagcctcccttttacgtgggctccaatggggatgactccttcagttccagcggggacctcagcttgtctgcttccccggtgcctgccagcctagcccagcctcctctccctgtcttaccaccattcccacccccgagtgggaagaatcccgtgatgatcttgaacgaactgcgcccaggactcaagtatgacttcctctccgagagcggggagagccatgccaagagcttcgtcatgtctgtggtcgtggatggtcagttctttgaaggctcggggagaaacaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccctga

SEQ ID NO: 166 - DNA, 632 bp, synthetic construct
  source 1..632: mol_type = other DNA
accggttttagtccctgaaggaagggactaaaataaagagtttgcgggactctgcggggttacaatcccctaaaaccgctaaggctagtccgttatcaacttgaaaaagtggcaccgagtcggtgcttttttgctagcctagacccagctttcttgtacaaagttggcattaatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctcca

SEQ ID NO: 167 - DNA, 19 bp, synthetic construct
  source 1..19: mol_type = other DNA
agggcgatgccacctgggg

SEQ ID NO: 168 - DNA, 19 bp, synthetic construct
  source 1..19: mol_type = other DNA
agggcgatgcgacctgggg

SEQ ID NO: 169 - DNA, 19 bp, synthetic construct
  source 1..19: mol_type = other DNA
agggcgatgggacctgggg

SEQ ID NO: 170 - DNA, 31 bp, synthetic construct
  source 1..31: mol_type = other DNA
atcgccctgaaaagggcgatgccacctgggg

SEQ ID NO: 171 - DNA, 31 bp, synthetic construct
  source 1..31: mol_type = other DNA
atcgccctgaaaagggcgatgcgacctgggg

SEQ ID NO: 172 - DNA, 31 bp, synthetic construct
  source 1..31: mol_type = other DNA
atcgccctgaaaagggcgatgggacctgggg

SEQ ID NO: 173 - DNA, 16 bp, synthetic construct
  source 1..16: mol_type = other DNA
accgctcagtttntaa

SEQ ID NO: 174 - DNA, 16 bp, synthetic construct
  source 1..16: mol_type = other DNA
ccgctcnntttntaaa

SEQ ID NO: 175 - DNA, 31 bp, synthetic construct
  source 1..31: mol_type = other DNA
gggcgatgccacctaaggcaagctgaccctg

SEQ ID NO: 176 - DNA, 19 bp, synthetic construct
  source 1..19: mol_type = other DNA
gccctaggtggcatcgccc

SEQ ID NO: 177 - DNA, 19 bp, synthetic construct
  source 1..19: mol_type = other DNA
cagggtcagcttgccctag

SEQ ID NO: 178 - DNA, 19 bp, synthetic construct
  source 1..19: mol_type = other DNA
gccccaggtggcatcgccc

SEQ ID NO: 179 - DNA, 19 bp, synthetic construct
  source 1..19: mol_type = other DNA
agggtcagcttgccccagg

SEQ ID NO: 180 - RNA, 66 nt, synthetic construct
  source 1..66: mol_type = other RNA
gtggaatagtataacaatatgctaaatgttgttatagtatcccacnnnnnncnnnnnnnnnnnnnn

SEQ ID NO: 181 - RNA, 68 nt, synthetic construct
  source 1..68: mol_type = other RNA
gggtggaatagtataacaatatgctaaatgttgttatagtatcccacctnnncnnnnnnnnnnnnnnn

SEQ ID NO: 182 - DNA, 24 bp, synthetic construct
  misc_feature 1..24: note = Description of Unknown:target sequence
  source 1..24: mol_type = other DNA
tcacagacaccgctcagtttgtaa

SEQ ID NO: 183 - DNA, 16 bp, synthetic construct
  source 1..16: mol_type = other DNA
caaaccgagcggtgtc

SEQ ID NO: 184 - DNA, 16 bp, synthetic construct
  source 1..16: mol_type = other DNA
acaaaccgagcggtgt

SEQ ID NO: 185 - DNA, 16 bp, synthetic construct
  source 1..16: mol_type = other DNA
tacaaaccgagcggtg

SEQ ID NO: 186 - DNA, 16 bp, synthetic construct
  source 1..16: mol_type = other DNA
ttacaaaccgagcggt

SEQ ID NO: 187 - DNA, 17 bp, synthetic construct
  source 1..17: mol_type = other DNA
caaaccgagcggtgtct

SEQ ID NO: 188 - DNA, 17 bp, synthetic construct
  source 1..17: mol_type = other DNA
acaaaccgagcggtgtc

SEQ ID NO: 189 - DNA, 17 bp, synthetic construct
  source 1..17: mol_type = other DNA
tacaaaccgagcggtgt

SEQ ID NO: 190 - DNA, 17 bp, synthetic construct
  source 1..17: mol_type = other DNA
ttacaaaccgagcggtg

SEQ ID NO: 191 - DNA, 18 bp, synthetic construct
  source 1..18: mol_type = other DNA
caaaccgagcggtgtctg

SEQ ID NO: 192 - DNA, 18 bp, synthetic construct
  source 1..18: mol_type = other DNA
acaaaccgagcggtgtct

SEQ ID NO: 193 - DNA, 18 bp, synthetic construct
  source 1..18: mol_type = other DNA
tacaaaccgagcggtgtc

SEQ ID NO: 194 - DNA, 18 bp, synthetic construct
  source 1..18: mol_type = other DNA
ttacaaaccgagcggtgt

SEQ ID NO: 195 - DNA, 19 bp, synthetic construct
  source 1..19: mol_type = other DNA
caaaccgagcggtgtctgt

SEQ ID NO: 196 - DNA, 19 bp, synthetic construct
  source 1..19: mol_type = other DNA
acaaaccgagcggtgtctg

SEQ ID NO: 197 - DNA, 19 bp, synthetic construct
  source 1..19: mol_type = other DNA
tacaaaccgagcggtgtct

SEQ ID NO: 198 - DNA, 19 bp, synthetic construct
  source 1..19: mol_type = other DNA
ttacaaaccgagcggtgtc

SEQ ID NO: 199 - DNA, 20 bp, synthetic construct
  source 1..20: mol_type = other DNA
caaaccgagcggtgtctgtg

SEQ ID NO: 200 - DNA, 20 bp, synthetic construct
  source 1..20: mol_type = other DNA
acaaaccgagcggtgtctgt

SEQ ID NO: 201 - DNA, 20 bp, synthetic construct
  source 1..20: mol_type = other DNA
tacaaaccgagcggtgtctg

SEQ ID NO: 202 - DNA, 20 bp, synthetic construct
  source 1..20: mol_type = other DNA
ttacaaaccgagcggtgtct

SEQ ID NO: 203 - DNA, 21 bp, synthetic construct
  source 1..21: mol_type = other DNA
caaaccgagcggtgtctgtga

SEQ ID NO: 204 - DNA, 21 bp, synthetic construct
  source 1..21: mol_type = other DNA
acaaaccgagcggtgtctgtg

SEQ ID NO: 205 - DNA, 21 bp, synthetic construct
  source 1..21: mol_type = other DNA
tacaaaccgagcggtgtctgt

SEQ ID NO: 206 - DNA, 21 bp, synthetic construct
  source 1..21: mol_type = other DNA
ttacaaaccgagcggtgtctg

SEQ ID NO: 207 - DNA, 14 bp, synthetic construct
  source 1..14: mol_type = other DNA
agcaataaaatggc

SEQ ID NO: 208 - DNA, 14 bp, synthetic construct
  source 1..14: mol_type = other DNA
agcaataaaatggc

SEQ ID NO: 209 - DNA, 39 bp, Mus sp.
  source 1..39: mol_type = other DNA
gagcaataaaatggcttcaactatctgagtgacactgtg

SEQ ID NO: 210 - DNA, 39 bp, Mus sp.; identical to SEQ ID NO: 209
  source 1..39: mol_type = other DNA

SEQ ID NO: 211 - DNA, 39 bp, synthetic construct
  source 1..39: mol_type = other DNA
gagccataaaatggcttcaactatctgagtgacactgtg

SEQ ID NO: 212 - DNA, 39 bp, synthetic construct
  source 1..39: mol_type = other DNA
gagcaataaaattgcttcaactatctgagtgacactgtg

SEQ ID NO: 213 - DNA, 39 bp, Mus sp.; identical to SEQ ID NO: 209
  source 1..39: mol_type = other DNA

SEQ ID NO: 214 - DNA, 39 bp, synthetic construct
  source 1..39: mol_type = other DNA
gagcaatggaatggcttcaactatctgagtgacactgtg

SEQ ID NO: 215 - DNA, 39 bp, synthetic construct; identical to SEQ ID NO: 211
  source 1..39: mol_type = other DNA

SEQ ID NO: 216 - DNA, 39 bp, synthetic construct; identical to SEQ ID NO: 212
  source 1..39: mol_type = other DNA

SEQ ID NO: 217 - DNA, 13 bp, synthetic construct
  source 1..13: mol_type = other DNA
ccgctcagtttnt

SEQ ID NO: 218 - DNA, 13 bp, synthetic construct
  source 1..13: mol_type = other DNA
ccgctcnntttnt

SEQ ID NO: 219 - DNA, 40 bp, Mus sp.
  source 1..40: mol_type = other DNA
tgaaagtctcacagacaccgctcatgtcttatctagcatg

SEQ ID NO: 220 - DNA, 40 bp, Mus sp.; identical to SEQ ID NO: 219
  source 1..40: mol_type = other DNA

SEQ ID NO: 221 - DNA, 40 bp, synthetic construct
  source 1..40: mol_type = other DNA
tgaaagtctcccagacaccgctcatgtcttatctagcatg

SEQ ID NO: 222 - DNA, 40 bp, synthetic construct
  source 1..40: mol_type = other DNA
tgcaagtctcacagacaccgctcatgtcttatctagcatg

SEQ ID NO: 223 - DNA, 40 bp, Mus sp.; identical to SEQ ID NO: 219
  source 1..40: mol_type = other DNA

SEQ ID NO: 224 - DNA, 40 bp, synthetic construct
  source 1..40: mol_type = other DNA
tgaaagtctcacagacaccgctcgtgtcttatctagcatg

SEQ ID NO: 225 - DNA, 40 bp, synthetic construct; identical to SEQ ID NO: 221
  source 1..40: mol_type = other DNA

SEQ ID NO: 226 - DNA, 40 bp, synthetic construct; identical to SEQ ID NO: 222
  source 1..40: mol_type = other DNA

SEQ ID NO: 227 - RNA, 66 nt, synthetic construct; identical to SEQ ID NO: 180
  source 1..66: mol_type = other RNA

SEQ ID NO: 228 - DNA, 39 bp, synthetic construct
  source 1..39: mol_type = other DNA
gccacctgaggcaagctgaccctgaagttcatctgcacc

SEQ ID NO: 229 - DNA, 39 bp, synthetic construct; identical to SEQ ID NO: 228
  source 1..39: mol_type = other DNA

SEQ ID NO: 230 - DNA, 39 bp, synthetic construct
  source 1..39: mol_type = other DNA
gccccctgaggcaagctgaccctgaagttcatctgcacc

SEQ ID NO: 231 - DNA, 39 bp, synthetic construct
  source 1..39: mol_type = other DNA
gccacctgaggcaagctgaccctgcagttcatctgcacc

SEQ ID NO: 232 - DNA, 39 bp, synthetic construct
  source 1..39: mol_type = other DNA
gccacctgaggccagctgaccctgaagttcatctgcacc

SEQ ID NO: 233 - DNA, 39 bp, synthetic construct
  source 1..39: mol_type = other DNA
gccacctgaggcaggctgaccctgaagttcatctgcacc

SEQ ID NO: 234 - DNA, 39 bp, synthetic construct; identical to SEQ ID NO: 228
  source 1..39: mol_type = other DNA

SEQ ID NO: 235 - DNA, 39 bp, synthetic construct
  source 1..39: mol_type = other DNA
gccacctgaggcaagctgaccctgaggttcatctgcacc

SEQ ID NO: 236 - DNA, 39 bp, synthetic construct
  source 1..39: mol_type = other DNA
gccgcctgaggcaagctgaccctgaagttcatctgcacc

SEQ ID NO: 237 - DNA, 39 bp, synthetic construct
  source 1..39: mol_type = other DNA
gccacctggggcaagctgaccctgaagttcatctgcacc

SEQ ID NO: 238 - DNA, 39 bp, synthetic construct; identical to SEQ ID NO: 233
  source 1..39: mol_type = other DNA

SEQ ID NO: 239 - DNA, 39 bp, synthetic construct
  source 1..39: mol_type = other DNA
gccacctgaggcgagctgaccctgaagttcatctgcacc

SEQ ID NO: 240 - DNA, 39 bp, synthetic construct
  source 1..39: mol_type = other DNA
gccgcctgaggcaagctgaccctgaggttcatctgcacc

SEQ ID NO: 241 - DNA, 39 bp, synthetic construct
  source 1..39: mol_type = other DNA
gccgcctgaggcaggctgaccctgaagttcatctgcacc

SEQ ID NO: 242 - DNA, 39 bp, synthetic construct; identical to SEQ ID NO: 228
  source 1..39: mol_type = other DNA

SEQ ID NO: 243 - DNA, 39 bp, synthetic construct; identical to SEQ ID NO: 237
  source 1..39: mol_type = other DNA

SEQ ID NO: 244 - DNA, 39 bp, synthetic construct; identical to SEQ ID NO: 232
  source 1..39: mol_type = other DNA

SEQ ID NO: 245 - DNA, 40 bp, synthetic construct
  source 1..40: mol_type = other DNA
cagacaccgctcagtttgtaaaacttttcttccttccaaa

SEQ ID NO: 246 - DNA, 40 bp, synthetic construct; identical to SEQ ID NO: 245
  source 1..40: mol_type = other DNA

SEQ ID NO: 247 - DNA, 40 bp, synthetic construct
  source 1..40: mol_type = other DNA
cagacaccgctcagtttgtaaaacttttcttccttccaca

SEQ ID NO: 248 - DNA, 40 bp, synthetic construct
  source 1..40: mol_type = other DNA
cagacaccgctcagtttgtaaaacttttcttccttccaac

SEQ ID NO: 249 - DNA, 40 bp, synthetic construct
  source 1..40: mol_type = other DNA
cagacaccgctcagtttgtgaaacttttcttccttccaaa

SEQ ID NO: 250 - DNA, 40 bp, synthetic construct
  source 1..40: mol_type = other DNA
cagacaccgctcagtttgtaacacttttcttccttccaaa

SEQ ID NO: 251 - DNA, 40 bp, synthetic construct
  source 1..40: mol_type = other DNA
cagacaccgctcagtttgtaaaccttttcttccttccaaa

SEQ ID NO: 252 - DNA, 40 bp, synthetic construct; identical to SEQ ID NO: 245
  source 1..40: mol_type = other DNA

SEQ ID NO: 253 - DNA, 40 bp, synthetic construct
  source 1..40: mol_type = other DNA
cagacaccgctcggtttgtaaaacttttcttccttccaaa

SEQ ID NO: 254 - DNA, 40 bp, synthetic construct; identical to SEQ ID NO: 249
  source 1..40: mol_type = other DNA

SEQ ID NO: 255 - DNA, 40 bp, synthetic construct
  source 1..40: mol_type = other DNA
cagacaccgctcggtttgtgaaacttttcttccttccaaa

SEQ ID NO: 256 - DNA, 40 bp, synthetic construct
  source 1..40: mol_type = other DNA
cagacaccgctcagtttgtagaacttttcttccttccaaa

SEQ ID NO: 257 - DNA, 40 bp, synthetic construct
  source 1..40: mol_type = other DNA
cagacaccgctcagtttgtaaaacttttcttccttccaga

SEQ ID NO: 258 - DNA, 40 bp, synthetic construct; identical to SEQ ID NO: 245
  source 1..40: mol_type = other DNA

SEQ ID NO: 259 - DNA, 40 bp, synthetic construct; identical to SEQ ID NO: 253
  source 1..40: mol_type = other DNA

SEQ ID NO: 260 - DNA, 40 bp, synthetic construct; identical to SEQ ID NO: 247
  source 1..40: mol_type = other DNA

SEQ ID NO: 261 - DNA, 40 bp, synthetic construct; identical to SEQ ID NO: 248
  source 1..40: mol_type = other DNA

SEQ ID NO: 262 - DNA, 40 bp, synthetic construct; identical to SEQ ID NO: 255
  source 1..40: mol_type = other DNA

SEQ ID NO: 263 - DNA, 40 bp, synthetic construct; identical to SEQ ID NO: 250
  source 1..40: mol_type = other DNA

SEQ ID NO: 264 - DNA, 40 bp, synthetic construct; identical to SEQ ID NO: 249
  source 1..40: mol_type = other DNA

SEQ ID NO: 265 - DNA, 62 bp, synthetic construct; identical to SEQ ID NO: 155
  source 1..62: mol_type = other DNA

SEQ ID NO: 266 - DNA, 62 bp, synthetic construct
  source 1..62: mol_type = other DNA
aacatgaggatcacccatgtctacaaaccgagcggtgtctgaacatgaggatcacccatgtc

SEQ ID NO: 267 - DNA, 62 bp, synthetic construct
  source 1..62: mol_type = other DNA
aacatgaggatcacccatgtctttacaaaccgagcggtgtcaacatgaggatcacccatgtc

SEQ ID NO: 268 - DNA, 62 bp, synthetic construct
  source 1..62: mol_type = other DNA
aacatgaggatcacccatgtcgttttacaaaccgagcggtgaacatgaggatcacccatgtc

SEQ ID NO: 269 - DNA, 62 bp, synthetic construct
  source 1..62: mol_type = other DNA
aacatgaggatcacccatgtcaagttttacaaaccgagcggaacatgaggatcacccatgtc

SEQ ID NO: 270 - protein, 5 aa, Mus sp.
TARVL

SEQ ID NO: 271 - DNA, 15 bp, Mus sp.
  source 1..15: mol_type = other DNA
  CDS 1..15
accgctcgtgtctta

SEQ ID NO: 272 - protein, 5 aa, synthetic construct
TAHVL

SEQ ID NO: 273 - DNA, 15 bp, synthetic construct
  source 1..15: mol_type = other DNA
  CDS 1..15
accgctcatgtctta

SEQ ID NO: 274 - protein, 21 aa, synthetic construct
TAQFVKLFFLPKFISNSDGVL

SEQ ID NO: 275 - DNA, 63 bp, synthetic construct
  source 1..63: mol_type = other DNA
  CDS 1..63
accgctcagtttgtaaaacttttcttccttccaaagtttatttcaaactctgatggtgtctta

(19) United States
(12) Patent Application Publication
(10) Pub. No.: US 2024/0392303 A1
(43) Pub. Date: Nov. 28, 2024

(54) RNA TARGETING OF MUTATIONS VIA SUPPRESSOR tRNAs AND DEAMINASES

(71) Applicant: The Regents of the University of California, Oakland, CA (US)

(72) Inventors: Prashant Mali, La Jolla, CA (US); Dhruva Katrekar, La Jolla, CA (US)

(21) Appl. No.: 18/786,394

(22) Filed: Jul. 26, 2024

Related U.S. Application Data
(63) Continuation of application No. 18/507,935, filed on Nov. 13, 2023.
Application No. 18/507,935 is a continuation of application No. 16/490,494, filed on Aug. 30, 2019, now Pat. No. 11,932,856.
(60) Provisional application No. 62/466,961, filed on Mar. 3, 2017.
(60) Provisional application No. 62/551,732, filed on Aug. 29, 2017.

(51) Int. Cl. C12N 15/115; A61K 38/00; A61P 21/00; C12N 9/78; C12N 15/113

(52) U.S. Cl. CPC C12N 15/115; A61P 21/00; C12N 9/78; C12Y 305/04; A61K 38/00; C12N 15/113; C12N 15/1137; C12N 2310/20; C12N 2310/531; C12N 2320/34

(57) ABSTRACT

Aspects of the disclosure relate to a gene therapy approach for diseases, disorders, or conditions caused by mutation in the stop codon utilizing modified tRNA. At least 10-15% of all genetic diseases, including muscular dystrophy (e.g. Duchene muscular dystrophy), some cancers, beta thalassemia, Hurler syndrome, and cystic fibrosis, fall into this category. Not to be bound by theory, it is believed that this approach is safer than CRISPR approaches due to minimal off-target effects and the lack of genome level changes.

### CROSS REFERENCE TO RELATED APPLICATION

This application is a continuation of U.S. application Ser. No. 18/507,935, filed Nov. 13, 2023, which is a continuation of U.S. application Ser. No. 16/490,494 (now U.S. Pat. No. 11,932,856), filed Aug. 30, 2019, which application is a U.S. National Stage Application filed under 35 U.S.C. § 371 and claims priority to International Application No. PCT/US2018/020762, filed Mar. 2, 2018, which claims priority under 35 U.S.C. 119 (c) to U.S. Ser. No. 62/466,961, filed Mar. 3, 2017, and U.S. Ser. No. 62/551,732, filed Aug. 29, 2017, the entirety of each of which are incorporated by reference herein.

### STATEMENT REGARDING GOVERNMENT SUPPORT

This invention was made with government support under Grant No. R01HG009285 awarded by the National Institutes of Health. The government has certain rights in the invention.

### SEQUENCE LISTING

The instant application contains a Sequence Listing which has been submitted electronically in ST.26.xml format and is hereby incorporated by reference in its entirety. Said xml copy, created on Jul. 26, 2024, is named “00015-364US8_SL.xml” and is 284,448 bytes in size.

### BACKGROUND

Aspects of the disclosure relate to a gene therapy approach for diseases, disorders, or conditions caused by mutation in the stop codon using modified tRNA. At least 10-15% of all genetic diseases, including muscular dystrophy (e.g. Duchene muscular dystrophy), some cancers, beta thalassemia, Hurler syndrome, and cystic fibrosis, fall into this category. Not to be bound by theory, it is believed that this approach is safer than CRISPR or TALEN approaches due to minimal off-target effects and the lack of genome level changes.

### SUMMARY

Aspects of the disclosure relate to a method for restoring expression of a protein comprising a point mutation in an RNA sequence encoding the protein in a subject in need thereof, the method comprising, or alternatively consisting essentially of, or yet further consisting of administering to the subject a vector encoding one or more tRNA having an anticodon sequence that recognizes a codon comprising the point mutation to the subject, optionally wherein the point mutation results in a premature stop codon, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding tRNA synthetase. In some embodiments, the corresponding synthetase is E. coli Glutaminyl-tRNA synthetase. In some embodiments involving an orthogonal tRNA, the non-canonical amino acid is pyrrolysine. In further embodiments, the pyrrolysine is administered to the subject by introduction into the diet of the subject. In some embodiments, the vector encodes two tRNA having an anticodon sequence that recognizes the codon comprising the point mutation. In some embodiments, the protein is dystrophin. In a further aspect, the subject is a human and is optionally a pediatric patient.

Further method aspects relate to a treating a disease, disorder, or condition characterized by the presence of a point mutation in an RNA sequence encoding a protein associated with the disease, disorder, or condition in a subject in need thereof, the method comprising, or alternatively consisting essentially of, or yet further consisting of, administering to the subject a vector encoding one or more tRNA having an anticodon sequence that recognizes a codon comprising the point mutation to the subject, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding tRNA synthetase. In some embodiments, the corresponding synthetase is E. coli Glutaminyl-tRNA synthetase. In some embodiments involving an orthogonal tRNA, the non-canonical amino acid is pyrrolysine. In further embodiments, the pyrrolysine is introduced in the diet of the subject. In some embodiments, the vector encodes two tRNA having an anticodon sequence that recognizes the codon comprising the point mutation. In some embodiments, the disease, disorder, or condition is selected from the group consisting of the diseases, disorders, and conditions listed in Table 1, optionally characterized by the presence of a nonsense mutation and/or a premature stop codon. In some embodiments, the protein is dystrophin. In further embodiments, the disease, disorder, or condition is muscular dystrophy. In still further embodiments, the disease disorder or condition is Duchenne muscular dystrophy. In some embodiments, the subject is a human and is optionally a pediatric patient.

Still further aspects disclosed herein relate to a vector encoding one or more tRNA having an anticodon sequence that recognizes a codon comprising a point mutation in an RNA sequence encoding a protein, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding IRNA synthetase. In some embodiments, the corresponding synthetase is E. coli Glutaminyl-tRNA synthetase. In some embodiments involving an orthogonal tRNA, the non-canonical amino acid is pyrrolysine. In some embodiments, the vector encodes two tRNA having an anticodon sequence that recognizes the codon comprising the point mutation. In some embodiments, the vector is an AAV vector, optionally an AAV8 vector. In some embodiments, the protein is dystrophin. In a further aspect, the subject is a human and is optionally a pediatric patient.

In another aspect, the disclosure relates to a method for restoring expression of a protein comprising a point mutation in an RNA sequence encoding the protein in a subject in need thereof comprising administering one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits the point mutation. In some embodiments, the point mutation results in a nonsense mutation having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, optionally those involving nonsense or missense mutations, the RNA targeted in mRNA. In further embodiments, the one or more vector further encodes a tRNA that targets an amber codon. In some embodiments, the protein is dystrophin. In some embodiments, the point mutation results in a splice site or missense mutation having the DNA sequence CAG and the RNA sequence CAG. In some embodiments, the ADAR based RNA editing system converts CAG to CIG. In some embodiments, optionally those involving splice site mutations, the RNA targeted is pre-mRNA. In some embodiments, the protein is ornithine transcabamylase. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the method further comprises administering an effective amount of an interferon to enhance endogenous ADAR1 expression. In still further embodiments, the interferon is interferon α. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA. In a further aspect, the subject is a human and is optionally a pediatric patient.

Further method aspects relate to a method of treating a disease, disorder, or condition characterized by the presence of a point mutation in an RNA sequence encoding a protein associated with the disease, disorder, or condition in a subject in need thereof, the method comprising, or alternatively consisting essentially of, or yet further consisting of, administering to the subject one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits the point mutation. In some embodiments, the point mutation results in a nonsense mutation having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, optionally those involving nonsense or missense mutations, the RNA targeted in mRNA. In further embodiments, the one or more vector further encodes a tRNA that targets an amber codon. In some embodiments, the protein is dystrophin. In some embodiments, the point mutation results in a splice site or missense mutation having the DNA sequence CAG and the RNA sequence CAG. In some embodiments, the ADAR based RNA editing system converts CAG to CIG. In some embodiments, optionally those involving splice site mutations, the RNA targeted is pre-mRNA. In some embodiments, the protein is ornithine transcabamylase. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the method further comprises administering an effective amount of an interferon to enhance endogenous ADAR1 expression. In still further embodiments, the interferon is interferon α. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA. In some embodiments, the disease, disorder, or condition is selected from the group consisting of the diseases, disorders, and conditions listed in Table 1. In further embodiments, the protein is dystrophin and the disease, disorder, or condition is muscular dystrophy. In still further embodiments, the disease disorder or condition is Duchenne muscular dystrophy. In some embodiments, the subject is a human and is optionally a pediatric patient.

Additional aspects relate to a recombinant expression system comprising one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits a point mutation in an RNA sequence encoding a protein. In some embodiments, the point mutation results in a nonsense mutation having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, optionally those involving nonsense or missense mutations, the RNA targeted in mRNA. In further embodiments, the one or more vector further encodes a tRNA that targets an amber codon. In some embodiments, the protein is dystrophin. In some embodiments, the point mutation results in a splice site or missense mutation having the DNA sequence CAG and the RNA sequence CAG. In some embodiments, the ADAR based RNA editing system converts CAG to CIG. In some embodiments, optionally those involving splice site mutations, the RNA targeted is pre-mRNA. In some embodiments, the protein is ornithine transcabamylase. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA. In a further aspect, the subject is a human and is optionally a pediatric patient.

Still further aspects relate to a composition comprising any one or more of the vectors disclosed herein and optionally one or more carriers, such as a pharmaceutically acceptable carrier. In some embodiments, the composition further comprises an effective amount of an interferon to enhance endogenous ADAR1 expression. In still further embodiments, the interferon is interferon α.

Some aspects disclosed herein relate to methods for restoring expression of a protein in a subject in need thereof, the method comprising, or alternatively consisting essentially of, or yet further consisting of, administering to the subject a tRNA having an anticodon sequence that recognizes a mutation in an RNA sequence encoding the protein or a vector encoding one or more of said tRNA to the subject. In some embodiments, the mutation is a nonsense mutation, optionally a premature stop codon. In some embodiments, the nonsense mutation is TAA in DNA and UAA in RNA. In some embodiments, the tRNA is a modified endogenous tRNA charged with a canonical amino acid. In some embodiments, the canonical amino acid is serine. In some embodiments, the tRNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the non-canonical amino acid is introduced or administered to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein is dystrophin. In a further aspect, the subject is a human and is optionally a pediatric patient.

Further aspects disclosed herein relate to methods of a disease, disorder, or condition characterized by a protein deficiency in a subject in need thereof, the method comprising, or alternatively consisting essentially or, or yet further consisting of administering a tRNA having an anticodon sequence that recognizes a mutation in an RNA sequence encoding the protein or a vector encoding one or more of said tRNA to the subject. In some embodiments, the mutation is a nonsense mutation, optionally a premature stop codon. In some embodiments, the nonsense mutation is TAA in DNA and UAA in RNA. In some embodiments, the tRNA is a modified endogenous tRNA charged with a canonical amino acid. In some embodiments, the canonical amino acid is serine. In some embodiments, the tRNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the non-canonical amino acid is administered or introduced to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein deficiency is a dystrophin deficiency. In some embodiments, the disease, disorder, or condition is muscular dystrophy. In some embodiments, the muscular dystrophy is Duchene muscular dystrophy. In a further aspect, the subject is a human and is optionally a pediatric patient.

Other aspects relate to a vector encoding one or more tRNA having an anticodon sequence that recognizes a mutation in an RNA sequence encoding the protein. In some embodiments, the mutation is a nonsense mutation, optionally a premature stop codon. In some embodiments, the nonsense mutation is TAA in DNA and UAA in RNA. In some embodiments, the tRNA is a modified endogenous tRNA charged with a canonical amino acid. In some embodiments, the canonical amino acid is serine. In some embodiments, the RNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the vector further comprises the corresponding synthetase. In some embodiments, the non-canonical amino acid is introduced or administered to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein is dystrophin. In some embodiments, the mutation is a nonsense mutation, optionally a premature stop codon. In some embodiments, the vector is an Adeno-Associated Virus (AAV) vector. In some embodiments, the AAV vector is an AAV8 vector.

Additional aspects of this disclosure relate to on-demand, in vivo production of therapeutic proteins, such as, but not limited to, (i) insulin; (ii) neutralizing antibodies for viruses (e.g. HIV, HCV, HPV, influenza) and bacteria (e.g. Staph aureus; drug resistant strains). Such method aspects comprise administering to a subject a vector encoding the therapeutic protein with a mutation in its sequence and a tRNA having an anticodon sequence that recognizes the mutation in the RNA sequence encoding the therapeutic protein or a vector encoding one or more of said tRNA. Accordingly, any of the methods and vectors disclosed hereinabove relating to a tRNA having an anticodon sequence that recognizes a mutation in an RNA sequence encoding the protein or a vector encoding one or more of said tRNA may be applied to this aspect, as well.

Some aspects disclosed herein relate to methods for restoring expression of a protein in a subject in need thereof comprising administering an ADAR2 based RNA editing system comprising an ADAR2, one or more forward guide RNAs for the ADAR2 (“adRNAs”), and one or more corresponding reverse guide RNAs for the ADAR2 (“radRNAs”) to the subject, wherein the ADAR2 based RNA editing system specifically edits a mutation in an RNA sequence encoding the protein or one or more vectors encoding said ADAR2, adRNAs, radRNAs. In some embodiments, the ADAR2 based RNA editing system changes adenosine (A) to inosine (I), which is read during translation as guanosine (G). In some embodiments, the mutation is a nonsense mutation. In some embodiments, the nonsense mutation is TAA in DNA and UAA in RNA. In some embodiments, the ADAR2 based RNA editing system causes point mutations at one or more adenosines (A) in the nonsense mutation. In some embodiments, the ADAR2 based RNA editing system converts UAA to UIA (read as UGA). In further embodiments, the ADAR2 based RNA editing system converts UIA (read as UGA) to UII (read as UGG). In some embodiments, the ADAR2 based RNA editing system converts UAA to UAI (read as UAG). In some embodiments, the method further comprises administering a tRNA, such as one disclosed hereinabove, that recognizes the codon encoded by the ADAR2 edited sequence. In some embodiments, the tRNA is a modified endogenous tRNA charged with a canonical amino acid. In some embodiments, the canonical amino acid is serine. In some embodiments, the tRNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the non-canonical amino acid is introduced to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein deficiency is a dystrophin deficiency. In some embodiments, the disease, disorder, or condition is muscular dystrophy. In some embodiments, the muscular dystrophy is Duchene muscular dystrophy.

Further aspects disclosed herein relate to methods of a disease, disorder, or condition characterized by a protein deficiency in a subject in need thereof comprising administering an ADAR2 based RNA editing system comprising an ADAR2, one or more forward guide RNAs for the ADAR2 (“adRNAs”), and one or more corresponding reverse guide RNAs for the ADAR2 (“radRNAs”) to the subject, wherein the ADAR2 based RNA editing system specifically edits a mutation in an RNA sequence encoding the protein or one or more vectors encoding said ADAR2, adRNAs, radRNAs. In some embodiments, the ADAR2 based RNA editing system changes adenosine (A) to inosine (I), which is read during translation as guanosine (G). In some embodiments, the mutation is a nonsense mutation. In some embodiments, the nonsense mutation is TAA. In some embodiments, the ADAR2 based RNA editing system causes point mutations at one or more adenosines (A) in the nonsense mutation. In some embodiments, the ADAR2 based RNA editing system converts UAA to UIA (read as UGA). In further embodiments, the ADAR2 based RNA editing system converts UIA (read as UGA) to UII (read as UGG). In some embodiments, the ADAR2 based RNA editing system converts UAA to UAI (read as UAG). In some embodiments, the method further comprises administering a tRNA, such as one disclosed hereinabove, that recognizes the codon encoded by the ADAR2 edited sequence. In some embodiments, the tRNA is a modified endogenous tRNA charged with a canonical amino acid. In some embodiments, the canonical amino acid is serine. In some embodiments, the tRNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the non-canonical amino acid is introduced to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein deficiency is a dystrophin deficiency. In some embodiments, the disease, disorder, or condition is muscular dystrophy. In some embodiments, the muscular dystrophy is Duchene muscular dystrophy.

Other aspects relate to a recombinant expression system comprising one or more vectors encoding an ADAR2 based RNA editing system comprising one or more of an ADAR2, one or more forward guide RNAs for the ADAR2 (“adRNAs”), and one or more corresponding reverse guide RNAs for the ADAR2 (“radRNAs”), wherein the ADAR2 based RNA editing system specifically edits a mutation in an RNA sequence encoding a protein. In some embodiments, the ADAR2 changes adenosine (A) to inosine (I), which is read during translation as guanosine (G). In some embodiments, one adRNA/radRNA pair guides the conversion of UAA to UIA (read as UGA). In further embodiments, a second adRNA/radRNA pair guides the conversion of UIA (read as UGA) to UII (read as UGG). In some embodiments, one adRNA/radRNA pair guides the conversion of UAA to UAI (read as UAG). In some embodiments, the one or more vectors or an additional vector further encodes a tRNA, such as one disclosed hereinabove, that recognizes the codon encoded by the ADAR2 edited sequence. In some embodiments, the tRNA is a modified endogenous tRNA charged with a canonical amino acid. In some embodiments, the canonical amino acid is serine. In some embodiments, the tRNA is an orthogonal tRNA charged with a non-canonical amino acid. In some embodiments, the orthogonal tRNA has a corresponding synthetase. In some embodiments, the corresponding synthetase is E. coli Glutaminyl-tRNA synthetase. In some embodiments, the non-canonical amino acid is introduced to the subject (e.g. through food), allowing for the induction of the orthogonal tRNA activity. In some embodiments, the non-canonical amino acid is pyrrolysine. In some embodiments, the tRNA targets an amber codon. In some embodiments, the tRNA targets an ochre codon. In some embodiments, the tRNA targets an opal codon. In some embodiments, the protein is dystrophin. In some embodiments, the mutation is a nonsense mutation. In some embodiments, the vector is an Adeno-Associated Virus (AAV) vector. In some embodiments, the AAV vector is an AAV8 vector.

Additional aspects of this disclosure relate to on-demand, in vivo production of therapeutic proteins, such as, but not limited to, (i) insulin; (ii) neutralizing antibodies for viruses (e.g. HIV, HCV, HPV, influenza) and bacteria (e.g. Staph aureus; drug resistant strains). Such method aspects comprise administering to a subject a vector encoding the therapeutic protein with a mutation in its sequence and an ADAR2 based RNA editing system comprising an ADAR2, one or more forward guide RNAs for the ADAR2 (“adRNAs”), and one or more corresponding reverse guide RNAs for the ADAR2 (“radRNAs”), wherein the ADAR2 based RNA editing system specifically edits a mutation in an RNA sequence encoding the protein or one or more vectors encoding said ADAR2, adRNAs, radRNAs. Accordingly, any of the methods and vectors disclosed hereinabove relating to an ADAR2 based RNA editing system specifically edits a mutation in an RNA sequence encoding the protein or a vector encoding one or more vectors encoding said ADAR2, adRNAs, radRNAs.

PARTIAL SEQUENCE LISTING

```
mU6, tRNA(U25C) Amber
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactCTAaatccgttcagccgggttagattcccggggttt
ccgccaTTTTTTCCTAGACCCAGCTTTCTTGTACAAAGTTGG (SEQ ID NO: 1)
mU6, tRNA(U25C) Ochre
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactTTAaatccgttcagccgggttagattcccggggttt
ccgccaTTTTTTCCTAGACCCAGCTTTCTTGTACAAAGTTGG (SEQ ID NO: 2)
mU6, tRNA(U25C) Opal
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactTCAaatccgttcagccgggttagattcccggggttt
ccgccaTTTTTTCCTAGACCCAGCTTTCTTGTACAAAGTTGG (SEQ ID NO: 3)
MmPylRS (AfIII)
CAGCCTCCGGACTCTAGAGGATCGAACCCTTAAGgccaccATGGATAAGAAACCTTTG
AACACTCTCATTAGTGCGACAGGGCTCTGGATGTCCCGAACGGGGACTATACACAA
GATAAAACACCATGAGGTCTCAAGGAGCAAAATCTATATCGAGATGGCATGCGGCG
ACCATCTTGTGGTAAATAATAGTAGGTCCTCCAGGACGGCAAGAGCACTCCGACATC
ACAAGTACAGAAAAACCTGCAAACGGTGTAGGGTATCCGACGAAGACTTGAACAAA
TTTTTGACTAAGGCCAACGAGGATCAAACTTCTGTCAAAGTGAAAGTGGTTTCTGCT
CCTACCCGAACTAAGAAGGCCATGCCCAAGTCCGTGGCAAGGGCACCCAAGCCACT
CGAAAATACTGAGGCCGCTCAGGCCCAACCATCCGGTAGTAAGTTCAGTCCAGCCA
TACCCGTAAGTACCCAAGAATCTGTCAGTGTGCCGGCCTCAGTTTCCACATCTATAA
GTTCAATTTCTACAGGAGCGACGGCCTCCGCCCTCGTCAAGGGTAACACAAACCCG
ATAACTTCTATGAGTGCCCCCGTACAGGCATCCGCACCAGCACTGACGAAGTCTCAA
ACTGATAGGCTGGAAGTGCTCTTGAATCCGAAGGACGAGATATCTCTTAACTCCGGT
AAACCTTTCCGGGAGCTGGAAAGTGAACTTCTCAGCCGGCGAAAAAAAGACCTCCA
GCAAATTTACGCAGAGGAAAGGGAGAACTATCTGGGGAAGTTGGAACGAGAGATC
ACCCGATTCTTTGTCGATCGCGGATTTTTGGAGATTAAAAGCCCAATTCTCATCCCCC
TTGAATATATCGAACGAATGGGAATCGACAATGATACGGAGTTGTCCAAGCAGATT
TTCCGCGTAGACAAGAACTTTTGTCTTCGACCCATGCTCGCTCCGAACCTCTACAATT
ACTTGAGAAAGTTGGACAGAGCGCTCCCGGACCCGATCAAGATATTTGAGATCGGT
CCTTGTTATAGAAAGGAGAGTGATGGAAAAGAACACCTCGAAGAGTTCACGATGCT
GAACTTCTGCCAAATGGGTTCTGGCTGCACACGGGAGAATCTCGAAAGCATCATTAC
AGATTTCCTTAACCATCTGGGGATAGACTTTAAAATAGTGGGTGACAGCTGTATGGT
ATACGGAGATACCTTGGACGTAATGCACGGGGATCTTGAGCTTTCCTCCGCCGTGGT
TGGACCTATACCGTTGGACCGGGAGTGGGGAATCGACAAACCGTGGATAGGCGCCG
GTTTCGGCCTTGAAAGACTCCTCAAAGTCAAGCATGATTTCAAAAACATAAAACGG
GCTGCTCGCTCCGAATCTTATTACAACGGTATAAGTACGAACCTGTGATAATAGCTT
AAGGGTTCGATCCCTACtGGTTAGTAATGAGTTTA (SEQ ID NO: 4)
tRNAs
Amber suppression:
ggaaacctgatcatgtagatcgaatggactctaaatccgttcagccgggttagattcccggggtttccgcca (SEQ ID NO: 5)
Amber suppression (2):
ggggggtggatcgaatagatcacacggactctaaattcgtgcaggcgggtgaaacteccgtactccccgcca (SEQ ID NO: 6)
Ochre supression
ggaaacctgatcatgtagatcgaatggactttaaatccgttcagccgggttagattcccggggtttccgcca (SEQ ID NO: 7)
Opal suppression:
ggaaacctgatcatgtagatcgaatggacttcaaatccgttcagccgggttagattcccggggtttccgcca (SEQ ID NO: 8)
Synthetase:
ATGGATAAAAAACCATTAGATGTTTTAATATCTGCGACCGGGCTCTGGATGTCCAGG
ACTGGCACGCTCCACAAAATCAAGCACCATGAGGTCTCAAGAAGTAAAATATACAT
TGAAATGGCGTGTGGAGACCATCTTGTTGTGAATAATTCCAGGAGTTGTAGAACAGC
CAGAGCATTCAGACATCATAAGTACAGAAAAACCTGCAAACGATGTAGGGTTTCGG
ACGAGGATATCAATAATTTTCTCAGAAGATCAACCGAAAGCAAAAACAGTGTGAAA
GTTAGGGTAGTTTCTGCTCCAAAGGTCAAAAAAGCTATGCCGAAATCAGTTTCAAGG
GCTCCGAAGCCTCTGGAAAATTCTGTTTCTGCAAAGGCATCAACGAACACATCCAGA
TCTGTACCTTCGCCTGCAAAATCAACTCCAAATTCGTCTGTTCCCGCATCGGCTCCTG
CTCCTTCACTTACAAGAAGCCAGCTTGATAGGGTTGAGGCTCTCTTAAGTCCAGAGG
ATAAAATTTCTCTAAATATGGCAAAGCCTTTCAGGGAACTTGAGCCTGAACTTGTGA
CAAGAAGAAAAAACGATTTTCAGCGGCTCTATACCAATGATAGAGAAGACTACCTC
GGTAAACTCGAACGTGATATTACGAAATTTTTCGTAGACCGGGGTTTTCTGGAGATA
AAGTCTCCTATCCTTATTCCGGCGGAATACGTGGAGAGAATGGGTATTAATAATGAT
ACTGAACT1TCAAAACAGATCTTCCGGGTGGATAAAAATCTCTGCTTGAGGCCAATG
CTTGCCCCGACTCTTTACAACTATCTGCGAAAACTCGATAGGATTTTACCAGGCCCA
ATAAAAATTTTCGAAGTCGOACCTTGTTACCOGAAAGAGTCTGACGGCAAAGAGCA
CCTGGAAGAATTTACTATGGTGAACTTCTGTCAGATGGGTTCGGGATGTACTCGGGA
AAATCTTGAAGCTCTCAICAAAGAGTTTCTGGACTATCTGGAAATCGACTTCGAAAT
CGTAGGAGATTCCTGTATGGTCTTTGGGGATACTCTTGATATAATGCACGGGGACCT
GGAGCTTTCTTCGGCAGTCGTCGGGCCAGTTTCTCTTGATAGAGAATGGGGTATTGA
CAAACCATGGATAGGTGCAGGTTTTGGTCTTGAACGCTTGCTCAAGGTTATGCACGG
CTTTAAAAACATTAAGAGGGCATCAAGGTCCGAATCTTACTATAATGGGATTTCAAC
CAATCTGTAA (SEQ ID NO: 9)
EGFP:
atggtgagcaagggcgaggagctgttcaccggggtggtgcccatcctggtcgagctggacggcgacgtaaacggecacaagttcagcgt
gtccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgc
ccgaaggctacgtccaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagctggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcgacggccccgtgctgctgcccgacaaccactacctgagcacccagtccgccct
gagcaaagaccccaacgagaagcgcgatcacatggtcctgctggagttcgtgaccgccgccgggatcactctcggcatggacgagctgt
acaagtaa (SEQ ID NO: 10)
EGFP Amber:
Atggtgagcaagggcgaggagctgttcaccggggtggtgcccatcctggtcgagctggacggcgacgtaaacggccacaagttcagcg
tgtccggcgagggcgagggcgatgccacctagggcaagctgaccctgaagttcatctgcaccaccggcaagctgcccgtgccctggcc
caccctcgtgaccaccctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaagttcgagggcgaca
ccctggtgaaccgcatcgagctgaagggcatcgacttcaaggaggacggcaacatcctggggcacaagctggagtacaactacaacagc
cacaacgtctatatcatggccgacaagcagaagaacggcatcaaggtgaacttcaagatccgccacaacatcgaggacggcagcgtgca
gctcgccgaccactaccagcagaacacccccatcggcgacggccccgtgctgctgcccgacaaccactacctgagcacccagtccgcc
ctgagcaaagaccccaacgagaagcgcgatcacatggtcctgctggagttcgtgaccgccgccgggatcactctcggcatggacgagct
gtacaagtaatga (SEQ ID NO: 11)
EGFP Ochre:
atggtgagcaagggcgaggagctgttcaccggggtggtgcccatcctggtcgagctggacggcgacgtaaacggccacaagttcagcgt
gtccggcgagggcgagggcgatgccacctaaggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgc
ccgaaggctacgtceaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagetggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcsacggccccgtgagctgcccgacaaccactacagagcacccagtccgccct
gagcaaagaccccaacgagaagcgcgatcacatggtcctgaggagttcgtgaccgccgccgggatcactetcggcatggacgagetgt
acaagtaatga (SEQ ID NO: 12)
EGFP Opa1:
Atggtgagcaagggcgaggagctgttcaccggggtggtgcccatcctggtcgagctggacggcgacgtaaacggccacaagttcagcg
tgtccggcgagggcgagggcgatgccacctgaggcaagctgaccctgaagttcatctgcaccaccggcaagctgcccgtgccctggcc
caccctcgtgaccaccctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaagttcgagggcgaca
ccctggtgaaccgcatcgagctgaagggcatcgacttcaaggaggacggcaacatcctggggcacaagctggagtacaactacaacagc
cacaacgtctatatcatggccgacaagcagaagaacggcatcaaggtgaacttcaagatccgccacaacatcgaggacggcagcgtgca
gctcgccgaccactaccagcagaacacccccatcggcgacggccccgtgctgctgcccgacaaccactacctgagcacccagtccgcc
ctgagcaaagaccccaacgagaagcgcgatcacatggtcctgctggagttcgtgaccgccgccgggatcactctcggcatggacgagct
gtacaagtaatga (SEQ ID NO: 13)
MbPylRS
                10 20 30 40 50
MDKKPLDVLI SATGLWMSRT GTLHKIKHHE VSRSKIYIEM ACGDHLVVNN
                60 70 80 90 100
SRSCRTARAF RHHKYRKTCK RCRVSDEDIN NFLTRSTESK NSVKVRVVSA
              110 120 130 140 150
PKVKKAMPKS VSRAPKPLEN SVSAKASTNT SRSVPSPAKS TPNSSVPASA
              160 170 180 190 200
PAPSLTRSQL DRVEALLSPE DKISLNMAKP FRELEPELVT RRKNDFQRLY
              210 220 230 240 250
TNDREDYLGK LERDITKFFV DRGFLEIKSP ILIPAEYVER MGINNDTELS
              260 270 280 290 300
KQIFRVDKNL CLRPMLAPTL YNYLRKLDRI LPGPIKIFEV GPCYRKESDG
              310 320 330 340 350
KEHLEEFTMV NFCQMGSGCT RENLEALIKE FLDYLEIDFE IVGDSCMVYG
              360 370 380 390 400
DTLDIMHGDL ELSSAVVGPV SLDREWGIDK PWIGAGFGLE RLLKVMHGFK
NIKRASRSES YYNGISTNL (SEQ ID NO: 14)
MmPylRS (uniprot)
                10 20 30 40 50
MDKKPLNTLI SATGLWMSRT GTIHKIKHHE VSRSKIYIEM ACGDHLVVNN
                60 70 80 90 100
SRSSRTARAL RHHKYRKTCK RCRVSDEDLN KFLTKANEDQ TSVKVKVVSA
              110 120 130 140 150
PTRTKKAMPK SVARAPKPLE NTEAAQAQPS GSKFSPAIPV STQESVSVPA
              160 170 180 190 200
SVSTSISSIS TGATASALVK GNTNPITSMS APVQASAPAL TKSQTDRLEV
              210 220 230 240 250
LLNPKDEISL NSGKPFRELE SELLSRRKKD LQQIYAEERE NYLGKLEREI
              260 270 280 290 300
TRFFVDRGFL EIKSPILIPL EYIERMGIDN DTELSKQIFR VDKNFCLRPM
              310 320 330 340 350
LAPNLYNYLR KLDRALPDPI KIFEIGPCYR KESDGKEHLE EFTMLNFCQM
              360 370 380 390 400
GSGCTRENLE SIITDFLNHL GIDFKIVGDS CMVYGDTLDV MHGDLELSSA
              410 420 430 440 450
VVGPIPLDRE WGIDKPWIGA GFGLERLLKV KHDFKNIKRA ARSESYYNGI
STNL (SEQ ID NO: 15)
PylT* (Amber)
ggaaacctgatcatgtagatcgaaCggactCTAaatccgttcagccgggttagattcccggggtttccgccaTTTTTT
(SEQ ID NO: 16)
PylT* (Ochre)
ggaaacctgatcatgtagatcgaaCggactTTAaatccgttcagccgggttagattcccggggTTTccgccaTTTTTT
(SEQ ID NO: 17)
PylT* (Opal)
ggaaacctgatcatgtagatcgaaCggactTCAaatccgttcagccgggttagattcccggggtttccgccaTTTTTT
(SEQ ID NO: 18)
Mouse U6 primers
tcccggggtttccgccaTTTTTTGGTACTGAGtCGCCCaGTCTCAGAT (SEQ ID NO: 19)
CAAACAAGGCTTTTCTCCAAGGGATAT (SEQ ID NO: 20)
tRNA (U25C) Amber_F:
CCTTGGAGAAAAGCCTTGTTTGggaaacctgatcatgtagatcgaacggactCTAaatccgttcagccggg
(SEQ ID NO: 21)
Common reverse:
PylT
ggaaacctgatcatgtagatcgaatggactCTAaatccgttcagccgggttagattcccggggTTTccgcca (SEQ ID
NO: 22)
PylT* (U25C)
ggaaacctgatcatgtagatcgaaCggactCTAaatccgttcagccgggttagattcccggggtttccgcca (SEQ ID
NO: 23)
1. Arg tRNA (opal) (E-Cadherin paper)
GGCCGCGTGGCCTAATGGATAAGGCGTCTGACTTCAGATCAGAAGATTGCAGGT
TCGAGTCCTGCCGCGGTCG (SEQ ID NO: 24)
2. Arg tRNA (opal) (Xeroderma paper)
GACCACGTGGCCTAATGGATAAGGCGTCTGACTTCAGATCAGAAGATTGAGGGT
TCGAATCCCTTCGTGGTTA (SEQ ID NO: 25)
3. Serine tRNA (amber)
GTAGTCGTGGCCGAGTGGTTAAGGCGATGGACTCTAAATCCATTGGGGTTTCCCC
GCGCAGGTTCGAATCCTGCCGACTACG (SEQ ID NO: 26)
4. Leucine tRNA (amber)
GTCAGGATGGCCGAGTGGTCTAAGGCGCCAGACTCTAGTTCTGGTCTCCAATGGA
GGCGTGGGTTCGAATCCCACTTCTGACA (SEQ ID NO: 27)
Forward:
TTGTGGAAAGGACGAAACACC (SEQ ID NO: 28)
Reverse:
ACAAGAAAGCTGGGTCTAGGCTAGCAAAAAA (SEQ ID NO: 29)
tRNA_Leu_Am_F (overlaps with vector, bold; anti-codon sequences, bold underline):
TTGTGGAAAGGACGAAACACCGGTCAGGATGGCCGAGTGGTCTAAGGCGCCAGAC
TCTAGTTCTGGTCTCCAATGG (SEQ ID NO: 30)
tRNA_Leu_Oc_F (overlaps with vector, bold; anti-codon sequences, bold underline):
TTGTGGAAAGGACGAAACACCGGTCAGGATGGCCGAGTGGTCTAAGGCGCCAGAC
TTTAGTTCTGGTCTCCAATGG (SEQ ID NO: 31)
tRNA_Leu_Op_F (overlaps with vector, bold; anti-codon sequences, bold underline):
TTGTGGAAAGGACGAAACACCGGTCAGGATGGCCGAGTGGTCTAAGGCGCCAGAC
TCTAGTTCTGGTCTCCAATGG (SEQ ID NO: 32)
tRNA_Leu_R (overlaps with vector, bold; anti-codon sequences, bold underline):
ACAAGAAAGCTGGGTCTAGGCTAGCAAAAAATGTCAGAAGTGGGATTCGAACCC
ACGCCTCCATTGGAGACCAGAAC (SEQ ID NO: 33)
tRNA_Ser_Am_F (overlaps with vector, bold; anti-codon sequences, bold underline):
TTGTGGAAAGGACGAAACACCGGTAGTCGTGGCCGAGTGGTTAAGGCGATGGACT
CTAAATCCATTGGGGTTTCC (SEQ ID NO: 34)
tRNA_Ser_Oc_F (overlaps with vector, bold; anti-codon sequences, bold underline):
TTGTGGAAAGGACGAAACACCGGTAGTCGTGGCCGAGTGGTTAAGGCGATGGACT
TTAAATCCATTGGGGTTTCC (SEQ ID NO: 35)
tRNA_Ser_Op (overlaps with vector, bold; anti-codon sequences, bold underline)F:
TTGTGGAAAGGACGAAACACCGGTAGTCGTGGCCGAGTGGTTAAGGCGATGGACT
TCAAATCCATTGGGGTTTCC (SEQ ID NO: 36)
tRNA_Ser_R (overlaps with vector, bold; anti-codon sequences, bold underline):
ACAAGAAAGCTGGGTCTAGGCTAGCAAAAAACGTAGTCGGCAGGATTCGAACCT
GCGCGGGGAAACCCCAATGGATT (SEQ ID NO: 37)
tRNA_Arg_Am_F (overlaps with vector, bold; anti-codon sequences, bold underline):
TTGTGGAAAGGACGAAACACCGGACCACGTGGCCTAATGGATAAGGCGTCTGACT
TCAGATCAGAAGATTGAGGGTT (SEQ ID NO: 38)
tRNA_Arg_Oc_F (overlaps with vector, bold; anti-codon sequences, bold underline):
TTGTGGAAAGGACGAAACACCGGACCACGTGGCCTAATGGATAAGGCGTCTGACT
TTAGATCAGAAGATTGAGGGTT (SEQ ID NO: 39)
tRNA_Arg_Op_F (overlaps with vector, bold; anti-codon sequences, bold underline):
TTGTGGAAAGGACGAAACACCGGACCACGTGGCCTAATGGATAAGGCGTCTGACT
TCAGATCAGAAGATTGAGGGTT (SEQ ID NO: 40)
tRNA_Arg_R (overlaps with vector, bold; anti-codon sequences, bold underline):
ACAAGAAAGCTGGGTCTAGGCTAGCAAAAAATAACCACGAAGGGATTCGAACCC
TCAATCTTCTGATC (SEQ ID NO: 41)
mU6_tRNA_ser_oc:
GTACTGAGtCGCCCaGTCTCAGATAGATCCGACGCCGCCATCTCTAGGCCCGCGCCGG
CCCCCTCGCACAGACTTGTGGGAGAAGCTCGGCTACTCCCCTGCCCCGGTTAATTTG
CATATAATATTTCCTAGTAACTATAGAGGCTTAATGTGCGATAAAAGACAGATAATC
TGTTCTTTTTAATACTAGCTACATTTTACATGATAGGCTTGGATTTCTATAAGAGATA
CAAATACTAAATTATTATTTTAAAAAACAGCACAAAAGGAAACTCACCCTAACTGTA
AAGTAATTGTGTGTTTTGAGACTATAAATATCCCTTGGAGAAAAGCCTTGTTTGGTA
GTCGTGGCCGAGTGGTTAAGGCGATGGACTTTAAATCCATTGGGGTTTCCCCGCGCA
GGTTCGAATCCTGCCGACTACGTTTTTT (SEQ ID NO: 42)
mU6_tRNA_ser_oc_Nhe1_insert_F:
AATCCTGCCGACTACGTTTTTTGTACTGAGtCGCCCAGTCT (SEQ ID NO: 43)
adRNA (premature stop codon target, bold; edited bases, bold underline):
Sequential edits:
TTTGAAAGAGCAATAAAAT (SEQ ID NO: 44)
CTTTGAAAGAGCAATAGAA (SEQ ID NO: 45)
Dual edits:
TTTGAAAGAGCAATAAAAT (SEQ ID NO: 46)
radRNA (premature stop codon target, bold; edited bases, bold underline):
Sequential edits:
AtaaAATGGCTTCAACTAT (SEQ ID NO: 47)
AAtagAATGGCTTCAACTA (SEQ ID NO: 48)
Dual edits:
AAtaaAATGGCTTCAACTA (SEQ ID NO: 49)
OTC target (edited bases, bold):
TCACAGACACCGCTCAGTTTGT (SEQ ID NO: 50)
Optimization of the length of adRNA and distance of the edit from the ADAR2
recruiting domain (Length of adRNA-distance of edit from ADAR2 recruiting domain):
16-5: atgccaccTGGggcaa (SEQ ID NO: 51)
16-6: tgccaccTGGggcaag (SEQ ID NO: 52)
16-7: gccaccTGGggcaagc (SEQ ID NO: 53)
18-6: gatgccaccTGGggcaag (SEQ ID NO: 54)
20-6: gcgatgccaccTGGggcaag (SEQ ID NO: 55)
ADAR2 recruiting region v1:
GGGTGGAATAGTATAACAATATGCTAAATGTTGTTATAGTATCCCACCT
(SEQ ID NO: 56)
ADAR2 recruiting region v2:
GTGGAATAGTATAACAATATGCTAAATGTTGTTATAGTATCCCAC (SEQ ID
NO: 57)
Hairpin (3′) (FIG. 8): GGGCCCTCTTCAGGGCCCTCTAGA (SEQ ID NO: 58)
Hairpin (3′) (FIG. 10): atcgccctgaaaag (SEQ ID NO: 59)
Toe hold (5′): gccaccTGGgg (SEQ ID NO: 60)
```

Optimization of the length of adRNA and distance of the edit from the ADAR2 recruiting domain (Length of adRNA—distance of edit from ADAR2 recruiting domain):

PARTIAL SEQUENCE LISTING

```
mU6, tRNA(U25C) Amber
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactCTAaatccgttcagccgggttagattcccggggttt
ccgccaTTTTTTCCTAGACCCAGCTTTCTTGTACAAAGTTGG (SEQ ID NO: 1)
mU6, tRNA(U25C) Ochre
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactTTAaatccgttcagccgggttagattcccggggttt
ccgccaTTTTTTCCTAGACCCAGCTTTCTTGTACAAAGTTGG (SEQ ID NO: 2)
mU6, tRNA(U25C) Opal
tcccggggtttccgccaTTTTTTGGTACTGAGtCGCCCaGTCTCAGATAGATCCGACGCCGCCAT
CTCTAGGCCCGCGCCGGCCCCCTCGCACAGACTTGTGGGAGAAGCTCGGCTACTCCC
CTGCCCCGGTTAATTTGCATATAATATTTCCTAGTAACTATAGAGGCTTAATGTGCG
ATAAAAGACAGATAATCTGTTCTTTTTAATACTAGCTACATTTTACATGATAGGCTTG
GATTTCTATAAGAGATACAAATACTAAATTATTATTTTAAAAAACAGCACAAAAGG
AAACTCACCCTAACTGTAAAGTAATTGTGTGTTTTGAGACTATAAATATCCCTTGGA
GAAAAGCCTTGTTTGggaaacctgatcatgtagatcgaaCggactTCAaatccgttcagccgggttagattcccggggttt
ccgccaTTTTTTCCTAGACCCAGCTTTCTTGTACAAAGTTGG (SEQ ID NO: 3)
MmPylRS (AfIII)
CAGCCTCCGGACTCTAGAGGATCGAACCCTTAAGgccaccATGGATAAGAAACCTTTG
AACACTCTCATTAGTGCGACAGGGCTCTGGATGTCCCGAACGGGGACTATACACAA
GATAAAACACCATGAGGTCTCAAGGAGCAAAATCTATATCGAGATGGCATGCGGCG
ACCATCTTGTGGTAAATAATAGTAGGTCCTCCAGGACGGCAAGAGCACTCCGACATC
ACAAGTACAGAAAAACCTGCAAACGGTGTAGGGTATCCGACGAAGACTTGAACAAA
TTTTTGACTAAGGCCAACGAGGATCAAACTTCTGTCAAAGTGAAAGTGGTTTCTGCT
CCTACCCGAACTAAGAAGGCCATGCCCAAGTCCGTGGCAAGGGCACCCAAGCCACT
CGAAAATACTGAGGCCGCTCAGGCCCAACCATCCGGTAGTAAGTTCAGTCCAGCCA
TACCCGTAAGTACCCAAGAATCTGTCAGTGTGCCGGCCTCAGTTTCCACATCTATAA
GTTCAATTTCTACAGGAGCGACGGCCTCCGCCCTCGTCAAGGGTAACACAAACCCG
ATAACTTCTATGAGTGCCCCCGTACAGGCATCCGCACCAGCACTGACGAAGTCTCAA
ACTGATAGGCTGGAAGTGCTCTTGAATCCGAAGGACGAGATATCTCTTAACTCCGGT
AAACCTTTCCGGGAGCTGGAAAGTGAACTTCTCAGCCGGCGAAAAAAAGACCTCCA
GCAAATTTACGCAGAGGAAAGGGAGAACTATCTGGGGAAGTTGGAACGAGAGATC
ACCCGATTCTTTGTCGATCGCGGATTTTTGGAGATTAAAAGCCCAATTCTCATCCCCC
TTGAATATATCGAACGAATGGGAATCGACAATGATACGGAGTTGTCCAAGCAGATT
TTCCGCGTAGACAAGAACTTTTGTCTTCGACCCATGCTCGCTCCGAACCTCTACAATT
ACTTGAGAAAGTTGGACAGAGCGCTCCCGGACCCGATCAAGATATTTGAGATCGGT
CCTTGTTATAGAAAGGAGAGTGATGGAAAAGAACACCTCGAAGAGTTCACGATGCT
GAACTTCTGCCAAATGGGTTCTGGCTGCACACGGGAGAATCTCGAAAGCATCATTAC
AGATTTCCTTAACCATCTGGGGATAGACTTTAAAATAGTGGGTGACAGCTGTATGGT
ATACGGAGATACCTTGGACGTAATGCACGGGGATCTTGAGCTTTCCTCCGCCGTGGT
TGGACCTATACCGTTGGACCGGGAGTGGGGAATCGACAAACCGTGGATAGGCGCCG
GTTTCGGCCTTGAAAGACTCCTCAAAGTCAAGCATGATTTCAAAAACATAAAACGG
GCTGCTCGCTCCGAATCTTATTACAACGGTATAAGTACGAACCTGTGATAATAGCTT
AAGGGTTCGATCCCTACtGGTTAGTAATGAGTTTA (SEQ ID NO: 4)
tRNAs
Amber suppression:
ggaaacctgatcatgtagatcgaatggactctaaatccgttcagccgggttagattcccggggtttccgcca (SEQ ID NO: 5)
Amber suppression (2):
ggggggtggatcgaatagatcacacggactctaaattcgtgcaggcgggtgaaacteccgtactccccgcca (SEQ ID NO: 6)
Ochre supression
ggaaacctgatcatgtagatcgaatggactttaaatccgttcagccgggttagattcccggggtttccgcca (SEQ ID NO: 7)
Opal suppression:
ggaaacctgatcatgtagatcgaatggacttcaaatccgttcagccgggttagattcccggggtttccgcca (SEQ ID NO: 8)
Synthetase:
ATGGATAAAAAACCATTAGATGTTTTAATATCTGCGACCGGGCTCTGGATGTCCAGG
ACTGGCACGCTCCACAAAATCAAGCACCATGAGGTCTCAAGAAGTAAAATATACAT
TGAAATGGCGTGTGGAGACCATCTTGTTGTGAATAATTCCAGGAGTTGTAGAACAGC
CAGAGCATTCAGACATCATAAGTACAGAAAAACCTGCAAACGATGTAGGGTTTCGG
ACGAGGATATCAATAATTTTCTCAGAAGATCAACCGAAAGCAAAAACAGTGTGAAA
GTTAGGGTAGTTTCTGCTCCAAAGGTCAAAAAAGCTATGCCGAAATCAGTTTCAAGG
GCTCCGAAGCCTCTGGAAAATTCTGTTTCTGCAAAGGCATCAACGAACACATCCAGA
TCTGTACCTTCGCCTGCAAAATCAACTCCAAATTCGTCTGTTCCCGCATCGGCTCCTG
CTCCTTCACTTACAAGAAGCCAGCTTGATAGGGTTGAGGCTCTCTTAAGTCCAGAGG
ATAAAATTTCTCTAAATATGGCAAAGCCTTTCAGGGAACTTGAGCCTGAACTTGTGA
CAAGAAGAAAAAACGATTTTCAGCGGCTCTATACCAATGATAGAGAAGACTACCTC
GGTAAACTCGAACGTGATATTACGAAATTTTTCGTAGACCGGGGTTTTCTGGAGATA
AAGTCTCCTATCCTTATTCCGGCGGAATACGTGGAGAGAATGGGTATTAATAATGAT
ACTGAACT1TCAAAACAGATCTTCCGGGTGGATAAAAATCTCTGCTTGAGGCCAATG
CTTGCCCCGACTCTTTACAACTATCTGCGAAAACTCGATAGGATTTTACCAGGCCCA
ATAAAAATTTTCGAAGTCGOACCTTGTTACCOGAAAGAGTCTGACGGCAAAGAGCA
CCTGGAAGAATTTACTATGGTGAACTTCTGTCAGATGGGTTCGGGATGTACTCGGGA
AAATCTTGAAGCTCTCAICAAAGAGTTTCTGGACTATCTGGAAATCGACTTCGAAAT
CGTAGGAGATTCCTGTATGGTCTTTGGGGATACTCTTGATATAATGCACGGGGACCT
GGAGCTTTCTTCGGCAGTCGTCGGGCCAGTTTCTCTTGATAGAGAATGGGGTATTGA
CAAACCATGGATAGGTGCAGGTTTTGGTCTTGAACGCTTGCTCAAGGTTATGCACGG
CTTTAAAAACATTAAGAGGGCATCAAGGTCCGAATCTTACTATAATGGGATTTCAAC
CAATCTGTAA (SEQ ID NO: 9)
EGFP:
atggtgagcaagggcgaggagctgttcaccggggtggtgcccatcctggtcgagctggacggcgacgtaaacggecacaagttcagcgt
gtccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgc
ccgaaggctacgtccaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagctggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcgacggccccgtgctgctgcccgacaaccactacctgagcacccagtccgccct
gagcaaagaccccaacgagaagcgcgatcacatggtcctgctggagttcgtgaccgccgccgggatcactctcggcatggacgagctgt
acaagtaa (SEQ ID NO: 10)
EGFP Amber:
Atggtgagcaagggcgaggagctgttcaccggggtggtgcccatcctggtcgagctggacggcgacgtaaacggccacaagttcagcg
tgtccggcgagggcgagggcgatgccacctagggcaagctgaccctgaagttcatctgcaccaccggcaagctgcccgtgccctggcc
caccctcgtgaccaccctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaagttcgagggcgaca
ccctggtgaaccgcatcgagctgaagggcatcgacttcaaggaggacggcaacatcctggggcacaagctggagtacaactacaacagc
cacaacgtctatatcatggccgacaagcagaagaacggcatcaaggtgaacttcaagatccgccacaacatcgaggacggcagcgtgca
gctcgccgaccactaccagcagaacacccccatcggcgacggccccgtgctgctgcccgacaaccactacctgagcacccagtccgcc
ctgagcaaagaccccaacgagaagcgcgatcacatggtcctgctggagttcgtgaccgccgccgggatcactctcggcatggacgagct
gtacaagtaatga (SEQ ID NO: 11)
EGFP Ochre:
atggtgagcaagggcgaggagctgttcaccggggtggtgcccatcctggtcgagctggacggcgacgtaaacggccacaagttcagcgt
gtccggcgagggcgagggcgatgccacctaaggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgc
ccgaaggctacgtceaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagetggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcsacggccccgtgagctgcccgacaaccactacagagcacccagtccgccct
gagcaaagaccccaacgagaagcgcgatcacatggtcctgaggagttcgtgaccgccgccgggatcactetcggcatggacgagetgt
acaagtaatga (SEQ ID NO: 12)
EGFP Opa1:
Atggtgagcaagggcgaggagctgttcaccggggtggtgcccatcctggtcgagctggacggcgacgtaaacggccacaagttcagcg
tgtccggcgagggcgagggcgatgccacctgaggcaagctgaccctgaagttcatctgcaccaccggcaagctgcccgtgccctggcc
caccctcgtgaccaccctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaagttcgagggcgaca
ccctggtgaaccgcatcgagctgaagggcatcgacttcaaggaggacggcaacatcctggggcacaagctggagtacaactacaacagc
cacaacgtctatatcatggccgacaagcagaagaacggcatcaaggtgaacttcaagatccgccacaacatcgaggacggcagcgtgca
gctcgccgaccactaccagcagaacacccccatcggcgacggccccgtgctgctgcccgacaaccactacctgagcacccagtccgcc
ctgagcaaagaccccaacgagaagcgcgatcacatggtcctgctggagttcgtgaccgccgccgggatcactctcggcatggacgagct
gtacaagtaatga (SEQ ID NO: 13)
MbPylRS
                10 20 30 40 50
MDKKPLDVLI SATGLWMSRT GTLHKIKHHE VSRSKIYIEM ACGDHLVVNN
                60 70 80 90 100
SRSCRTARAF RHHKYRKTCK RCRVSDEDIN NFLTRSTESK NSVKVRVVSA
              110 120 130 140 150
PKVKKAMPKS VSRAPKPLEN SVSAKASTNT SRSVPSPAKS TPNSSVPASA
              160 170 180 190 200
PAPSLTRSQL DRVEALLSPE DKISLNMAKP FRELEPELVT RRKNDFQRLY
              210 220 230 240 250
TNDREDYLGK LERDITKFFV DRGFLEIKSP ILIPAEYVER MGINNDTELS
              260 270 280 290 300
KQIFRVDKNL CLRPMLAPTL YNYLRKLDRI LPGPIKIFEV GPCYRKESDG
              310 320 330 340 350
KEHLEEFTMV NFCQMGSGCT RENLEALIKE FLDYLEIDFE IVGDSCMVYG
              360 370 380 390 400
DTLDIMHGDL ELSSAVVGPV SLDREWGIDK PWIGAGFGLE RLLKVMHGFK
NIKRASRSES YYNGISTNL (SEQ ID NO: 14)
MmPylRS (uniprot)
                10 20 30 40 50
MDKKPLNTLI SATGLWMSRT GTIHKIKHHE VSRSKIYIEM ACGDHLVVNN
                60 70 80 90 100
SRSSRTARAL RHHKYRKTCK RCRVSDEDLN KFLTKANEDQ TSVKVKVVSA
              110 120 130 140 150
PTRTKKAMPK SVARAPKPLE NTEAAQAQPS GSKFSPAIPV STQESVSVPA
              160 170 180 190 200
SVSTSISSIS TGATASALVK GNTNPITSMS APVQASAPAL TKSQTDRLEV
              210 220 230 240 250
LLNPKDEISL NSGKPFRELE SELLSRRKKD LQQIYAEERE NYLGKLEREI
              260 270 280 290 300
TRFFVDRGFL EIKSPILIPL EYIERMGIDN DTELSKQIFR VDKNFCLRPM
              310 320 330 340 350
LAPNLYNYLR KLDRALPDPI KIFEIGPCYR KESDGKEHLE EFTMLNFCQM
              360 370 380 390 400
GSGCTRENLE SIITDFLNHL GIDFKIVGDS CMVYGDTLDV MHGDLELSSA
              410 420 430 440 450
VVGPIPLDRE WGIDKPWIGA GFGLERLLKV KHDFKNIKRA ARSESYYNGI
STNL (SEQ ID NO: 15)
PylT* (Amber)
ggaaacctgatcatgtagatcgaaCggactCTAaatccgttcagccgggttagattcccggggtttccgccaTTTTTT
(SEQ ID NO: 16)
PylT* (Ochre)
ggaaacctgatcatgtagatcgaaCggactTTAaatccgttcagccgggttagattcccggggTTTccgccaTTTTTT
(SEQ ID NO: 17)
PylT* (Opal)
ggaaacctgatcatgtagatcgaaCggactTCAaatccgttcagccgggttagattcccggggtttccgccaTTTTTT
(SEQ ID NO: 18)
Mouse U6 primers
tcccggggtttccgccaTTTTTTGGTACTGAGtCGCCCaGTCTCAGAT (SEQ ID NO: 19)
CAAACAAGGCTTTTCTCCAAGGGATAT (SEQ ID NO: 20)
tRNA (U25C) Amber_F:
CCTTGGAGAAAAGCCTTGTTTGggaaacctgatcatgtagatcgaacggactCTAaatccgttcagccggg
(SEQ ID NO: 21)
Common reverse:
PylT
ggaaacctgatcatgtagatcgaatggactCTAaatccgttcagccgggttagattcccggggTTTccgcca (SEQ ID
NO: 22)
PylT* (U25C)
ggaaacctgatcatgtagatcgaaCggactCTAaatccgttcagccgggttagattcccggggtttccgcca (SEQ ID
NO: 23)
1. Arg tRNA (opal) (E-Cadherin paper)
GGCCGCGTGGCCTAATGGATAAGGCGTCTGACTTCAGATCAGAAGATTGCAGGT
TCGAGTCCTGCCGCGGTCG (SEQ ID NO: 24)
2. Arg tRNA (opal) (Xeroderma paper)
GACCACGTGGCCTAATGGATAAGGCGTCTGACTTCAGATCAGAAGATTGAGGGT
TCGAATCCCTTCGTGGTTA (SEQ ID NO: 25)
3. Serine tRNA (amber)
GTAGTCGTGGCCGAGTGGTTAAGGCGATGGACTCTAAATCCATTGGGGTTTCCCC
GCGCAGGTTCGAATCCTGCCGACTACG (SEQ ID NO: 26)
4. Leucine tRNA (amber)
GTCAGGATGGCCGAGTGGTCTAAGGCGCCAGACTCTAGTTCTGGTCTCCAATGGA
GGCGTGGGTTCGAATCCCACTTCTGACA (SEQ ID NO: 27)
Forward:
TTGTGGAAAGGACGAAACACC (SEQ ID NO: 28)
Reverse:
ACAAGAAAGCTGGGTCTAGGCTAGCAAAAAA (SEQ ID NO: 29)
tRNA_Leu_Am_F (overlaps with vector, bold; anti-codon sequences, bold underline):
TTGTGGAAAGGACGAAACACCGGTCAGGATGGCCGAGTGGTCTAAGGCGCCAGAC
TCTAGTTCTGGTCTCCAATGG (SEQ ID NO: 30)
tRNA_Leu_Oc_F (overlaps with vector, bold; anti-codon sequences, bold underline):
TTGTGGAAAGGACGAAACACCGGTCAGGATGGCCGAGTGGTCTAAGGCGCCAGAC
TTTAGTTCTGGTCTCCAATGG (SEQ ID NO: 31)
tRNA_Leu_Op_F (overlaps with vector, bold; anti-codon sequences, bold underline):
TTGTGGAAAGGACGAAACACCGGTCAGGATGGCCGAGTGGTCTAAGGCGCCAGAC
TCTAGTTCTGGTCTCCAATGG (SEQ ID NO: 32)
tRNA_Leu_R (overlaps with vector, bold; anti-codon sequences, bold underline):
ACAAGAAAGCTGGGTCTAGGCTAGCAAAAAATGTCAGAAGTGGGATTCGAACCC
ACGCCTCCATTGGAGACCAGAAC (SEQ ID NO: 33)
tRNA_Ser_Am_F (overlaps with vector, bold; anti-codon sequences, bold underline):
TTGTGGAAAGGACGAAACACCGGTAGTCGTGGCCGAGTGGTTAAGGCGATGGACT
CTAAATCCATTGGGGTTTCC (SEQ ID NO: 34)
tRNA_Ser_Oc_F (overlaps with vector, bold; anti-codon sequences, bold underline):
TTGTGGAAAGGACGAAACACCGGTAGTCGTGGCCGAGTGGTTAAGGCGATGGACT
TTAAATCCATTGGGGTTTCC (SEQ ID NO: 35)
tRNA_Ser_Op (overlaps with vector, bold; anti-codon sequences, bold underline)F:
TTGTGGAAAGGACGAAACACCGGTAGTCGTGGCCGAGTGGTTAAGGCGATGGACT
TCAAATCCATTGGGGTTTCC (SEQ ID NO: 36)
tRNA_Ser_R (overlaps with vector, bold; anti-codon sequences, bold underline):
ACAAGAAAGCTGGGTCTAGGCTAGCAAAAAACGTAGTCGGCAGGATTCGAACCT
GCGCGGGGAAACCCCAATGGATT (SEQ ID NO: 37)
tRNA_Arg_Am_F (overlaps with vector, bold; anti-codon sequences, bold underline):
TTGTGGAAAGGACGAAACACCGGACCACGTGGCCTAATGGATAAGGCGTCTGACT
TCAGATCAGAAGATTGAGGGTT (SEQ ID NO: 38)
tRNA_Arg_Oc_F (overlaps with vector, bold; anti-codon sequences, bold underline):
TTGTGGAAAGGACGAAACACCGGACCACGTGGCCTAATGGATAAGGCGTCTGACT
TTAGATCAGAAGATTGAGGGTT (SEQ ID NO: 39)
tRNA_Arg_Op_F (overlaps with vector, bold; anti-codon sequences, bold underline):
TTGTGGAAAGGACGAAACACCGGACCACGTGGCCTAATGGATAAGGCGTCTGACT
TCAGATCAGAAGATTGAGGGTT (SEQ ID NO: 40)
tRNA_Arg_R (overlaps with vector, bold; anti-codon sequences, bold underline):
ACAAGAAAGCTGGGTCTAGGCTAGCAAAAAATAACCACGAAGGGATTCGAACCC
TCAATCTTCTGATC (SEQ ID NO: 41)
mU6_tRNA_ser_oc:
GTACTGAGtCGCCCaGTCTCAGATAGATCCGACGCCGCCATCTCTAGGCCCGCGCCGG
CCCCCTCGCACAGACTTGTGGGAGAAGCTCGGCTACTCCCCTGCCCCGGTTAATTTG
CATATAATATTTCCTAGTAACTATAGAGGCTTAATGTGCGATAAAAGACAGATAATC
TGTTCTTTTTAATACTAGCTACATTTTACATGATAGGCTTGGATTTCTATAAGAGATA
CAAATACTAAATTATTATTTTAAAAAACAGCACAAAAGGAAACTCACCCTAACTGTA
AAGTAATTGTGTGTTTTGAGACTATAAATATCCCTTGGAGAAAAGCCTTGTTTGGTA
GTCGTGGCCGAGTGGTTAAGGCGATGGACTTTAAATCCATTGGGGTTTCCCCGCGCA
GGTTCGAATCCTGCCGACTACGTTTTTT (SEQ ID NO: 42)
mU6_tRNA_ser_oc_Nhe1_insert_F:
AATCCTGCCGACTACGTTTTTTGTACTGAGtCGCCCAGTCT (SEQ ID NO: 43)
adRNA (premature stop codon target, bold; edited bases, bold underline):
Sequential edits:
TTTGAAAGAGCAATAAAAT (SEQ ID NO: 44)
CTTTGAAAGAGCAATAGAA (SEQ ID NO: 45)
Dual edits:
TTTGAAAGAGCAATAAAAT (SEQ ID NO: 46)
radRNA (premature stop codon target, bold; edited bases, bold underline):
Sequential edits:
AtaaAATGGCTTCAACTAT (SEQ ID NO: 47)
AAtagAATGGCTTCAACTA (SEQ ID NO: 48)
Dual edits:
AAtaaAATGGCTTCAACTA (SEQ ID NO: 49)
OTC target (edited bases, bold):
TCACAGACACCGCTCAGTTTGT (SEQ ID NO: 50)
Optimization of the length of adRNA and distance of the edit from the ADAR2
recruiting domain (Length of adRNA-distance of edit from ADAR2 recruiting domain):
16-5: atgccaccTGGggcaa (SEQ ID NO: 51)
16-6: tgccaccTGGggcaag (SEQ ID NO: 52)
16-7: gccaccTGGggcaagc (SEQ ID NO: 53)
18-6: gatgccaccTGGggcaag (SEQ ID NO: 54)
20-6: gcgatgccaccTGGggcaag (SEQ ID NO: 55)
ADAR2 recruiting region v1:
GGGTGGAATAGTATAACAATATGCTAAATGTTGTTATAGTATCCCACCT
(SEQ ID NO: 56)
ADAR2 recruiting region v2:
GTGGAATAGTATAACAATATGCTAAATGTTGTTATAGTATCCCAC (SEQ ID
NO: 57)
Hairpin (3′) (FIG. 8): GGGCCCTCTTCAGGGCCCTCTAGA (SEQ ID NO: 58)
Hairpin (3′) (FIG. 10): atcgccctgaaaag (SEQ ID NO: 59)
Toe hold (5′): gccaccTGGgg (SEQ ID NO: 60)
```

List of suppressor tRNA sequences:

| Suppressor | |
|---|---|
| tRNAs | Sequence (5′ to 3′) |
| Serine | GTAGTCGTGGCCGAGTGGTTAAGGCGATGGACTNNNAATCCATTGG |
| | GGTTTCCCCGCGCAGGTTCGAATCCTGCCGACTACG (SEQ ID NO: 61) |
| Leucine | GTCAGGATGGCCGAGTGGTCTAAGGCGCCAGACTNNNGTTCTGGTC |
| | TCCAATGGAGGCGTGGGTTCGAATCCCACTTCTGACA (SEQ ID NO: 62) |
| Arginine | GACCACGTGGCCTAATGGATAAGGCGTCTGACTNNNGATCAGAAG |
| | ATTGAGGGTTCGAATCCCTTCGTGGTTA (SEQ ID NO: 63) |

### NNN—Anticodon

In endogenous tRNA, the tRNA is modified to recognize the codon comprising the point mutation by including the complementary sequence at the NNN position noted herein above. As clarified in more detail below, the NNN sequences in amber, ochre, and opal tRNA are as follows: Amber: NNN=CTA; Ochre: NNN=TCA; Opal: NNN=TTA.

List of primers for next generation sequencing (NGS) analyses.

| Name | Sequence (5′ to 3′) |
|---|---|
| NGS_DMD_F1 | GTGTTACTGAATATGAAATAATGGAGGA (SEQ ID |
| | NO: 64) |
| NGS_DMD_R1 | ATTTCTGGCATATTTCTGAAGGTG (SEQ ID NO: |
| | 65) |
| NGS_DMD_F2 | CTCTCTGTACCTTATCTTAGTGTTACTGA (SEQ ID |
| | NO: 66) |
| NGS_DMD_R2 | CTCTTCAAATTCTGACAGATATTTCTGGC (SEQ ID |
| | NO: 67) |
| NGS_OTC_F | ACCCTTCCTTTCTTACCACACA (SEQ ID NO: 68) |
| NGS_OTC_R | CAGGGTGTCCAGATCTGATTGTT (SEQ ID NO: |
| | 69) |
| NGS_OTC_R2 | CTTCTCTTTTAAACTAACCCATCAGAGTT (SEQ ID |
| | NO: 70) |

List of adRNA antisense sequences and corresponding ADAR2 recruiting scaffold used for in vivo RNA editing studies. In some embodiments, the recruiting scaffold v2—disclosed in the preceding paragraph, is used with these sequences.

| Name | adRNA antisense sequence (3′ to 5′) |
|---|---|
| OTC | TGTCTGTGGCGAGCCAAACA (SEQ ID NO: 71) |
| DMD | ACTTTCTCGTTACCTTACCG (SEQ ID NO: 72) |

MCP-Linker-ADAR1-NLS (optional sequence in brackets)

MCP-Linker-ADAR1-NLS (optional sequence in brackets)
(SEQ ID NO: 73)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSKAERMGFTEVTPVTGASLRRTMLLLSRSPEA
QPKTLPLTGSTFHDQIAMLSHRCFNTLTNSFQPSLLGRKILAAIIMKKDSEDMGVVVSLG
TGNRCVKGDSLSLKGETVNDCHAEIISRRGFIRFLYSELMKYNSQTAKDSIFEPAKGGEK
LQIKKTVSFHLYISTAPCGDGALFDKSCSDRAMESTESRHYPVFENPKQGKLRTKVENG
EGTIPVESSDIVPTWDGIRLGERLRTMSCSDKILRWNVLGLQGALLTHFLQPIYLKSVTLG
YLFSQGHLTRAICCRVTRDGSAFEDGLRHPFIVNHPKVGRVSIYDSKRQSGKTKETSVN
WCLADGYDLEILDGTRGTVDGPRNELSRVSKKNIFLLFKKLCSFRYRRDLLRLSYGEAK
KAARDYETAKNYFKKGLKDMGYGNWISKPQEEKNFYLCPVGSGSGSGPKKRKV[AA]*
MCP-Linker-ADAR2 (optional sequence in brackets)
(SEQ ID NO: 74)
MGPKKKRKVAAGSGSGSMASNFTQFVLVDNGGTGDVTVAPSNFANGVAEWISSNSR
SQAYKVTCSVRQSSAQKRKYTIKVEVPKVATQTVGGVELPVAAWRSYLNMELTIPI
FATNSDCELIVKAMQGLLKDGNPIPSAIAANSGIYGGSGGSGGSMLHLDQTPSRQPIPS
EGLQLHLPQVLADAVSRLVLGKFGDLTDNFSSPHARRKVLAGVVMTTGTDVKDAKVIS
VSTGTKCINGEYMSDRGLALNDCHAEIISRRSLLRFLYTQLELYLNNKDDQKRSIFQKSE
RGGFRLKENVQFHLYISTSPCGDARIFSPHEPILEEPADRHPNRKARGQLRTKIESGEGTIP
VRSNASIQTWDGVLQGERLLTMSCSDKIARWNVVGIQGSLLSIFVEPIYFSSIILGSLYHG
DHLSRAMYQRISNIEDLPPLYTLNKPLLSGISNAEARQPGKAPNFSVNWTVGDSAIEVIN
ATTGKDELGRASRLCKHALYCRWMRVHGKVPSHLLRSKITKPNVYHESKLAAKEYQA
AKARLFTAFIKAGLGAWVEKPTEQDQFSLT[P]*
N22p-Linker-ADAR1-NLS (optional sequence in brackets)
(SEQ ID NO: 75)
MGNARTRRRERRAEKQAQWKAANGGGGTSGSGSGSPAGGGAPGSGGGSKAERMGF
TEVTPVTGASLRRTMLLLSRSPEAQPKTLPLTGSTFHDQIAMLSHRCFNTLTNSFQPSLLG
RKILAAIIMKKDSEDMGVVVSLGTGNRCVKGDSLSLKGETVNDCHAEIISRRGFIRFLYS
ELMKYNSQTAKDSIFEPAKGGEKLQIKKTVSFHLYISTAPCGDGALFDKSCSDRAMESTE
SRHYPVFENPKQGKLRTKVENGEGTIPVESSDIVPTWDGIRLGERLRTMSCSDKILRWNV
LGLQGALLTHFLQPIYLKSVTLGYLFSQGHLTRAICCRVTRDGSAFEDGLRHPFIVNHPK
VGRVSIYDSKRQSGKTKETSVNWCLADGYDLEILDGTRGTVDGPRNELSRVSKKNIFLL
FKKLCSFRYRRDLLRLSYGEAKKAARDYETAKNYFKKGLKDMGYGNWISKPQEEKNF
YLCPVGSGSGSGPKKRKV[AA]*
Nuclear Localization Sequence-Linker-N22p-Linker-ADAR2
(optional sequence in brackets)
(SEQ ID NO: 76)
[MG]PKKKRKVAAGSGSGSMGNARTRRRERRAEKQAQWKAANGGGGTSGSGSGSP
AGGGAPGSGGGSMLHLDQTPSRQPIPSEGLQLHLPQVLADAVSRLVLGKFGDLTDNFSSP
HARRKVLAGVVMTTGTDVKDAKVISVSTGTKCINGEYMSDRGLALNDCHAEIISRRSLL
RFLYTQLELYLNNKDDQKRSIFQKSERGGFRLKENVQFHLYISTSPCGDARIFSPHEPILE
EPADRHPNRKARGQLRTKIESGEGTIPVRSNASIQTWDGVLQGERLLTMSCSDKIARWN
VVGIQGSLLSIFVEPIYFSSIILGSLYHGDHLSRAMYQRISNIEDLPPLYTLNKPLLSGISNA
EARQPGKAPNFSVNWTVGDSAIEVINATTGKDELGRASRLCKHALYCRWMRVHGKVP
SHLLRSKITKPNVYHESKLAAKEYQAAKARLFTAFIKAGLGAWVEKPTEQDQFSLT[P]*
MCP-Linker-ADAR1(E1008Q)-NLS (optional sequence in brackets)
(SEQ ID NO: 77)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSKAERMGFTEVTPVTGASLRRTMLLLSRSPEA
QPKTLPLTGSTFHDQIAMLSHRCFNTLTNSFQPSLLGRKILAAIIMKKDSEDMGVVVSLG
TGNRCVKGDSLSLKGETVNDCHAEIISRRGFIRFLYSELMKYNSQTAKDSIFEPAKGGEK
LQIKKTVSFHLYISTAPCGDGALFDKSCSDRAMESTESRHYPVFENPKQGKLRTKVENG
QGTIPVESSDIVPTWDGIRLGERLRTMSCSDKILRWNVLGLQGALLTHFLQPIYLKSVTL
GYLFSQGHLTRAICCRVTRDGSAFEDGLRHPFIVNHPKVGRVSIYDSKRQSGKTKETSVN
WCLADGYDLEILDGTRGTVDGPRNELSRVSKKNIFLLFKKLCSFRYRRDLLRLSYGEAK
KAARDYETAKNYFKKGLKDMGYGNWISKPQEEKNFYLCPVGSGSGSGPKKRKV[AA]*
Nuclear Localization Sequence-Linker-MCP-Linker-ADAR2(E488Q) (optional
sequence in brackets)
(SEQ ID NO: 78)
[MG]PKKKRKVAAGSGSGSMASNFTQFVLVDNGGTGDVTVAPSNFANGVAEWISSNS
RSQAYKVTCSVRQSSAQKRKYTIKVEVPKVATQTVGGVELPVAAWRSYLNMELTI
PIFATNSDCELIVKAMQGLLKDGNPIPSAIAANSGIYGGSGGSGGSMLHLDQTPSRQPI
PSEGLQLHLPQVLADAVSRLVLGKFGDLTDNFSSPHARRKVLAGVVMTTGTDVKDAKV
ISVSTGTKCINGEYMSDRGLALNDCHAEIISRRSLLRFLYTQLELYLNNKDDQKRSIFQKS
ERGGFRLKENVQFHLYISTSPCGDARIFSPHEPILEEPADRHPNRKARGQLRTKIESGQGT
IPVRSNASIQTWDGVLQGERLLTMSCSDKIARWNVVGIQGSLLSIFVEPIYFSSIILGSLYH
GDHLSRAMYQRISNIEDLPPLYTLNKPLLSGISNAEARQPGKAPNFSVNWTVGDSAIEVI
NATTGKDELGRASRLCKHALYCRWMRVHGKVPSHLLRSKITKPNVYHESKLAAKEYQ
AAKARLFTAFIKAGLGAWVEKPTEQDQFSLT[P]*
N22p-Linker-ADAR1(E1008Q) (optional sequence in brackets)
(SEQ ID NO: 79)
MGNARTRRRERRAEKQAQWKAANGGGGTSGSGSGSPAGGGAPGSGGGSKAERMGF
TEVTPVTGASLRRTMLLLSRSPEAQPKTLPLTGSTFHDQIAMLSHRCFNTLTNSFQPSLLG
RKILAAIIMKKDSEDMGVVVSLGTGNRCVKGDSLSLKGETVNDCHAEIISRRGFIRFLYS
ELMKYNSQTAKDSIFEPAKGGEKLQIKKTVSFHLYISTAPCGDGALFDKSCSDRAMESTE
SRHYPVFENPKQGKLRTKVENGQGTIPVESSDIVPTWDGIRLGERLRTMSCSDKILRWN
VLGLQGALLTHFLQPIYLKSVTLGYLFSQGHLTRAICCRVTRDGSAFEDGLRHPFIVNHP
KVGRVSIYDSKRQSGKTKETSVNWCLADGYDLEILDGTRGTVDGPRNELSRVSKKNIFL
LFKKLCSFRYRRDLLRLSYGEAKKAARDYETAKNYFKKGLKDMGYGNWISKPQEEKN
FYLCPVGSGSGSGPKKRKV[AA]*
Nuclear Localization Sequence-Linker-N22p-Linker-ADAR2(E488Q)
(SEQ ID NO: 80)
[MG]PKKKRKVAAGSGSGSMGNARTRRRERRAEKQAQWKAANGGGGTSGSGSGSP
AGGGAPGSGGGSMLHLDQTPSRQPIPSEGLQLHLPQVLADAVSRLVLGKFGDLTDNFSSP
HARRKVLAGVVMTTGTDVKDAKVISVSTGTKCINGEYMSDRGLALNDCHAEIISRRSLL
RFLYTQLELYLNNKDDQKRSIFQKSERGGFRLKENVQFHLYISTSPCGDARIFSPHEPILE
EPADRHPNRKARGQLRTKIESGQGTIPVRSNASIQTWDGVLQGERLLTMSCSDKIARWN
VVGIQGSLLSIFVEPIYFSSIILGSLYHGDHLSRAMYQRISNIEDLPPLYTLNKPLLSGISNA
EARQPGKAPNFSVNWTVGDSAIEVINATTGKDELGRASRLCKHALYCRWMRVHGKVP
SHLLRSKITKPNVYHESKLAAKEYQAAKARLFTAFIKAGLGAWVEKPTEQDQFSLT[P]*
Nuclear Localization Sequence-Linker-MCP-Linker-hAPOPEC1
(SEQ ID NO: 81)
[MG]PKKKRKVAAGSGSGSMASNFTQFVLVDNGGTGDVTVAPSNFANGVAEWISSNS
RSQAYKVTCSVRQSSAQKRKYTIKVEVPKVATQTVGGVELPVAAWRSYLNMELTI
PIFATNSDCELIVKAMQGLLKDGNPIPSAIAANSGIYGGSGGSGGSMTSEKGPSTGDPT
LRRRIEPWEFDVFYDPRELRKEACLLYEIKWGMSRKIWRSSGKNTTNHVEVNFIKKFTSE
RDFHPSMSCSITWFLSWSPCWECSQAIREFLSRHPGVTLVIYVARLFWHMDQQNRQGLR
DLVNSGVTIQIIVIRASEYYHCWRNFVNYPPGDEAHWPQYPPLWMMLYALELHCIILSLPP
CLKISRRWQNHLTFFRLHLQNCHYQTIPPHILLATGLIHPSVAWR*
Nuclear Localization Sequence-Linker-MCP-Linker-rAPOBEC1
(SEQ ID NO: 82)
[MG]PKKKRKVAAGSGSGSMASNFTQFVLVDNGGTGDVTVAPSNFANGVAEWISSNS
RSQAYKVTCSVRQSSAQKRKYTIKVEVPKVATQTVGGVELPVAAWRSYLNMELTI
PIFATNSDCELIVKAMQGLLKDGNPIPSAIAANSGIYGGSGGSGGSMSSETGPVAVDPT
LRRRIEPHEFEVFFDPRELRKETCLLYEINWGGRHSIWRHTSCINTNKHVEVNFIEKFTTER
YFCPNTRCSITWFLSWSPCGECSRAITEFLSRYPHVTLFIYIARLYHHADPRNRQGLRDLI
SSGVTIQIMTEQESGYCWRNFVNYSPSNEAHWPRYPHLWVRLYVLELYCIILGLPPCLNI
LRRKQPQLTFFTIALCISCHYQRLPPHILWATGLK*
dsRBD-Linker-rAPOBEC1
(SEQ ID NO: 83)
MDIEDEENMSSSSTDVKENRNLDNVSPKDGSTPGPGEGSQLSNGGGGGPGRKRPLE
EGSNGHSKYRLKKRRKTPGPVLPKNALMQLNEIKPGLQYTLLSQTGPVHAPLFVM
SVEVNGQVFEGSGPTKKKAKLHAAEKALRSFVQFPNASEAHLAMGRTLSVNTDFT
SDQADFPDTLFNGFETPDKAEPPFYVGSNGDDSFSSSGDLSLSASPVPASLAQPPLPV
LPPFPPPSGKNPVMILNELRPGLKYDFLSESGESHAKSFVMSVVVDGQFFEGSGRNK
KLAKARAAQSALAAIFNGGSGGSGGSMSSETGPVAVDPTLRRRIEPHEFEVFFDPRELR
KETCLLYEINWGGRHSIWRHTSQNTNKHVEVNFIEKFTTERYFCPNTRCSITWFLSWSPC
GECSRAITEFLSRYPHVTLFIYIARLYHHADPRNRQGLRDLISSGVTIQIMTEQESGYCWR
NFVNYSPSNEAHWPRYPHLWVRLYVLELYCIILGLPPCLNILRRKQPQLTFFTIALQSCH
YQRLPPHILWATGLK*
dsRBD-Linker-hAPOBEC1
(SEQ ID NO: 84)
MDIEDEENMSSSSTDVKENRNLDNVSPKDGSTPGPGEGSQLSNGGGGGPGRKRPLE
EGSNGHSKYRLKKRRKTPGPVLPKNALMQLNEIKPGLQYTLLSQTGPVHAPLFVM
SVEVNGQVFEGSGPTKKKAKLHAAEKALRSFVQFPNASEAHLAMGRTLSVNTDFT
SDQADFPDTLFNGFETPDKAEPPFYVGSNGDDSFSSSGDLSLSASPVPASLAQPPLPV
LPPFPPPSGKNPVMILNELRPGLKYDFLSESGESHAKSFVMSVVVDGQFFEGSGRNK
KLAKARAAQSALAAIFNGGSGGSGGSMTSEKGPSTGDPTLRRRIEPWEFDVFYDPRELR
KEACLLYEIKWGMSRKIWRSSGKNTTNHVEVNFIKKFTSERDFHPSMSCSITWFLSWSPC
WECSQAIREFLSRHPGVTLVIYVARLFWHMDQQNRQGLRDLVNSGVTIQIMRASEYYH
CWRNFVNYPPGDEAHWPQYPPLWMMLYALELHCIILSLPPCLKISRRWQNHLTFFRLHL
QNCHYQTIPPHILLATGLIHPSVAWR*
MCP-Linker-ADAR1-NES
(SEQ ID NO: 85)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSKAERMGFTEVTPVTGASLRRTMLLLSRSPEA
QPKTLPLTGSTFHDQIAMLSHRCFNTLTNSFQPSLLGRKILAAIIMKKDSEDMGVVVSLG
TGNRCVKGDSLSLKGETVNDCHAEIISRRGFIRFLYSELMKYNSQTAKDSIFEPAKGGEK
LQIKKTVSFHLYISTAPCGDGALFDKSCSDRAMESTESRHYPVFENPKQGKLRTKVENG
EGTIPVESSDIVPTWDGIRLGERLRTMSCSDKILRWNVLGLQGALLTHFLQPIYLKSVTLG
YLFSQGHLTRAICCRVTRDGSAFEDGLRHPFIVNHPKVGRVSIYDSKRQSGKTKETSVN
WCLADGYDLEILDGTRGTVDGPRNELSRVSKKNIFLLFKKLCSFRYRRDLLRLSYGEAK
KAARDYETAKNYFKKGLKDMGYGNWISKPQEEKNFYLCPVGSGSGSLPPLERLTL*
MCP-Linker-ADAR2-NLS
(SEQ ID NO: 86)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSQLHLPQVLADAVSRLVLGKFGDLTDNFSSPH
ARRKVLAGVVMTTGTDVKDAKVISVSTGTKCINGEYMSDRGLALNDCHAEIISRRSLLR
FLYTQLELYLNNKDDQKRSIFQKSERGGFRLKENVQFHLYISTSPCGDARIFSPHEPILEEP
ADRHPNRKARGQLRTKIESGEGTIPVRSNASIQTWDGVLQGERLLTMSCSDKIARWNVV
GIQGSLLSIFVEPIYFSSIILGSLYHGDHLSRAMYQRISNIEDLPPLYTLNKPLLSGISNAEA
RQPGKAPNFSVNWTVGDSAIEVINATTGKDELGRASRLCKHALYCRWMRVHGKVPSH
LLRSKITKPNVYHESKLAAKEYQAAKARLFTAFIKAGLGAWVEKPTEQDQFSLTGSGSG
SPKKKRKV*
MCP-Linker-ADAR2-NES
(SEQ ID NO: 87)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSQLHLPQVLADAVSRLVLGKFGDLTDNFSSPH
ARRKVLAGVVMTTGTDVKDAKVISVSTGTKCINGEYMSDRGLALNDCHAEIISRRSLLR
FLYTQLELYLNNKDDQKRSIFQKSERGGFRLKENVQFHLYISTSPCGDARIFSPHEPILEEP
ADRHPNRKARGQLRTKIESGEGTIPVRSNASIQTWDGVLQGERLLTMSCSDKIARWNVV
GIQGSLLSIFVEPIYFSSIILGSLYHGDHLSRAMYQRISNIEDLPPLYTLNKPLLSGISNAEA
RQPGKAPNFSVNWTVGDSAIEVINATTGKDELGRASRLCKHALYCRWMRVHGKVPSH
LLRSKITKPNVYHESKLAAKEYQAAKARLFTAFIKAGLGAWVEKPTEQDQFSLTGSGSG
SLPPLERLTL*
MCP-Linker-rAPOBEC1-NLS
(SEQ ID NO: 88)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSSGSETPGTSESATPESMSSETGPVAVDPTLRR
RIEPHEFEVFFDPRELRKETCLLYEINWGGRHSIWRHTSQNTNKHVEVNFIEKFT
TERYFCPNTRCSITWFLSWSPCGECSRAITEFLSRYPHVTLFIYIARLYHHADPRNRQGLR
DLISSGVTIQIMTEQESGYCWRNFVNYSPSNEAHWPRYPHLWVRLYVLELYCIILGLPPC
LNILRRKQPQLTFFTIALCISCHYQRLPPHILWATGLKGSGSGSPKKKRKV*
MCP-Linker-rAPOBEC1-NES
(SEQ ID NO: 89)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSSGSETPGTSESATPESMSSETGPVAVDPTLRR
RIEPHEFEVFFDPRELRKETCLLYEINWGGRHSIWRHTSQNTNKHVEVNFIEKFTTERYFC
PNTRCSITWFLSWSPCGECSRAITEFLSRYPHVTLFIYIARLYHHADPRNRQGLRDLISSG
VTIQIMTEQESGYCWRNFVNYSPSNEAHWPRYPHLWVRLYVLELYCIILGLPPCLNILRR
KQPQLTFFTIALQSCHYQRLPPHILWATGLKGSGSGSLPPLERLTL*
MCP-Linker-hAPOBEC1-NLS
(SEQ ID NO: 90)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSSGSETPGTSESATPESMTSEKGPSTGDPTLRRR
IEPWEFDVFYDPRELRKEACLLYEIKWGMSRKIWRSSGKNTTNHVEVNFIKKFTSERDF
HPSMSCSITWFLSWSPCWECSCIAIREFLSRHPGVTLVIYVARLFWHMDQQNRQGLRDLV
NSGVTIQIMRASEYYHCWRNFVNYPPGDEAHWPQYPPLWMMLYALELHCIILSLPPCLK
ISRRWQNHLTFFRLHLCINCHYQTIPPHILLATGLIHPSVAWRGSGSGSPKKKRKV*
MCP-Linker-hAPOBEC1-NES
(SEQ ID NO: 91)
MASNFTQFVLVDNGGTGDVTVAPSNFANGIAEWISSNSRSQAYKVTCSVRQSSAQN
RKYTIKVEVPKGAWRSYLNMELTIPIFATNSDCELIVKAMQGLLKDGNPIPSAIAAN
SGIYGGSGSGAGSGSPAGGGAPGSGGGSSGSETPGTSESATPESMTSEKGPSTGDPTLRRR
IEPWEFDVFYDPRELRKEACLLYEIKWGMSRKIWRSSGKNTTNHVEVNFIKKFTSERDF
HPSMSCSITWFLSWSPCWECSQAIREFLSRHPGVTLVIYVARLFWHMDQQNRQGLRDLV
NSGVTIQIMRASEYYHCWRNFVNYPPGDEAHWPQYPPLWMMLYALELHCIILSLPPCLK
ISRRWQNHLTFFRLHLQNCHYQTIPPHILLATGLIHPSVAWRGSGSGSLPPLERLTL*
Alternate spacer (can be used in place of GGSGGSGGS
(SEQ ID NO: 92)):
(SEQ ID NO: 93)
SGSETPGTSESATPES
3XNLS-4xlN-cdADAR2
(SEQ ID NO: 94)
MPKKKRKVDPKKKRKVDPKKKRKVGSYPYDVPDYAGSNARTRRRERRAEKQAQ
WKAANGGGGSGGGGSGGGGSNARTRRRERRAEKQAQWKAANGGGGSGGGGSG
GGGSNARTRRRERRAEKQAQWKAANGGGGSGGGGSGGGGSNARTRRRERRAEK
QAQWKAANLHLDQTPSRQPIPSEGLQLHLPQVLADAVSRLVLGKFGDLTDNFSSPHAR
RKVLAGVVMTTGTDVKDAKVISVSTGTKCINGEYMSDRGLALNDCHAEIISRRSLLRFL
YTQLELYLNNKDDQKRSIFQKSERGGFRLKENVQFHLYISTSPCGDARIFSPHEPILEEPA
DRHPNRKARGQLRTKIESGEGTIPVRSNASIQTWDGVLQGERLLTMSCSDKIARWNVVG
IQGSLLSIFVEPIYFSSIILGSLYHGDHLSRAMYQRISNIEDLPPLYTLNKPLLSGISNAEAR
QPGKAPNFSVNWTVGDSAIEVINATTGKDELGRASRLCKHALYCRWMRVHGKVPSHLL
RSKITKPNVYHESKLAAKEYQAAKARLFTAFIKAGLGAWVEKPTEQDQFSLTP
N22p-hAPOBEC1
(SEQ ID NO: 95)
MPKKKRKVDGSGNARTRRRERRAEKQAQWKAANGGGGTSGSGSGSPAGGGAPG
SGGGSMTSEKGPSTGDPTLRRRIEPWEFDVFYDPRELRKEACLLYEIKWGMSRKIWRSS
GKNTTNHVEVNFIKKFTSERDFHPSMSCSITWFLSWSPCWECSQAIREFLSRHPGVTLVI
YVARLFWHMDQQNRQGLRDLVNSGVTIQIMRASEYYHCWRNFVNYPPGDEAHWPQY
PPLWMMLYALELHCIILSLPPCLKISRRWQNHLTFFRLHLQNCHYQTIPPHILLATGLIHP
SVAWR
3XNLS-4xlN-hAPOBEC1
(SEQ ID NO: 96)
MPKKKRKVDPKKKRKVDPKKKRKVGSYPYDVPDYAGSNARTRRRERRAEKQAQ
WKAANGGGGSGGGGSGGGGSNARTRRRERRAEKQAQWKAANGGGGSGGGGSG
GGGSNARTRRRERRAEKQAQWKAANGGGGSGGGGSGGGGSNARTRRRERRAEK
QAQWKAANMTSEKGPSTGDPTLRRRIEPWEFDVFYDPRELRKEACLLYEIKWGMSRKI
WRSSGKNTTNHVEVNFIKKFTSERDFHPSMSCSITWFLSWSPCWECSQAIREFLSRHPGV
TLVIYVARLFWHMDQQNRQGLRDLVNSGVTIQIMRASEYYHCWRNFVNYPPGDEAHW
PQYPPLWMMLYALELHCIILSLPPCLKISRRWQNHLTFFRLHLQNCHYQTIPPHILLATGL
IHPSVAWR
C-terminal ADAR2 (residues 1-138 deleted)
(SEQ ID NO: 97)
MLRSFVQFPNASEAHLAMGRTLSVNTDFTSDQADFPDTLFNGFETPDKAEPPFYVGSNG
DDSFSSSGDLSLSASPVPASLAQPPLPVLPPFPPPSGKNPVMILNELRPGLKYDFLSESGES
HAKSFVMSVVVDGQFFEGSGRNKKLAKARAAQSALAAIFNLHLDQTPSRQPIPSEGLQL
HLPQVLADAVSRLVLGKFGDLTDNFSSPHARRKVLAGVVMTTGTDVKDAKVISVSTGT
KCINGEYMSDRGLALNDCHAEIISRRSLLRFLYTQLELYLNNKDDQKRSIFQKSERGGFR
LKENVQFHLYISTSPCGDARIFSPHEPILEEPADRHPNRKARGQLRTKIESGEGTIPVRSNA
SIQTWDGVLQGERLLTMSCSDKIARWNVVGIQGSLLSIFVEPIYFSSIILGSLYHGDHLSR
AMYQRISNIEDLPPLYTLNKPLLSGISNAEARQPGKAPNFSVNWTVGDSAIEVINATTGK
DELGRASRLCKHALYCRWMRVHGKVPSHLLRSKITKPNVYHESKLAAKEYQAAKARL
FTAFIKAGLGAWVEKPTEQDQFSLTP*
MS2-RNA:
Single:
(SEQ ID NO: 98)
NNNNNNNNNNNNNNNNNNNNggccAACATGAGGATCACCCATGTCTGCAGggcc
Dual:
(SEQ ID NO: 99)
aACATGAGGATCACCCATGTcNNNNNNNNNNNNNNNNNNNNaACATGAGGATCACC
CATGTc
BoxB RNA:
Single:
(SEQ ID NO: 100)
NNNNNNNNNNNNNNNNNNNNgggccctgaagaagggccc
Dual:
(SEQ ID NO: 101)
ggGCCCTGAAGAAGGGCccNNNNNNNNNNNNNNNNNNNNggGCCCTGAAGAAGGGCcc
PP7-RNA:
(SEQ ID NO: 102)
NNNNNNNNNNNNNNNNNNNNccggagcagacgatatggcgtcgctccgg
Dual Hairpin RNA:
(SEQ ID NO: 103)
TGGAATAGTATAACAATATGCTAAATGTTGTTATAGTATCCCAC
NNNNNNNGTGGAATAGTATAACAATATGCTAAATGTTGTTATAGTATCCCAC
A-U to G-C substitutions in adRNA
v1:
(SEQ ID NO: 104)
GGGTGGAATAGTATAACAATATGCTAAATGTTGTTATAGTATCCCACCT
NNNCNNNNNNNNNNNNNNN
v2:
(SEQ ID NO: 105)
GTGGAATAGTATAACAATATGCTAAATGTTGTTATAGTATCCCAC
NNNNNNCNNNNNNNNNNNNN
v3:
(SEQ ID NO: 106)
GTGGAAGAGGAGAACAATATGCTAAATGTTGTTCTCGTCTCCCAC
NNNNNNCNNNNNNNNNNNNNN
v4:
(SEQ ID NO: 107)
GGGTGGAAGAGGAGAACAATATGCTAAATGTTGTTCTCGTCTCCCACCT
NNNCNNNNNNNNNNNNNNN
v5:
(SEQ ID NO: 108)
GGTGAAGAGGAGAACAATATGCTAAATGTTGTTCTCGTCTCCACC
NNNNNNCNNNNNNNNNNNNNN
v6:
(SEQ ID NO: 109)
GGTGAAGAGGAGAACAATATGCTAAATGTTGTTCTCGTCTCCACC
NNNNNNNCNNNNNNNNNNNNN
v7:
(SEQ ID NO: 110)
GTGGAAGAGGAGAACAATAGGCTAAACGTTGTTCTCGTCTCCCAC
NNNNNNCNNNNNNNNNNNNNN
v8:
(SEQ ID NO: 111)
GGGTGGAAGAGGAGAACAATAGGCTAAACGTTGTTCTCGTCTCCCACCT
NNNCNNNNNNNNNNNNNNN
v9:
(SEQ ID NO: 112)
GGTGAAGAGGAGAACAATAGCTAAACGTTGTTCTCGTCTCCACC
NNNNNNCNNNNNNNNNNNNNN
v10:
(SEQ ID NO: 113)
GGTGAAGAGGAGAACAATAGCTAAACGTTGTTCTCGTCTCCACC
NNNNNNNCNNNNNNNNNNNN
v11:
(SEQ ID NO: 114)
GGTGTCGAGAATAGTATAACCAATATGCTAAATGTTGTTATAGTATCCTCGACACC
NNNNNNNCNNNNNNNNNN
v12:
(SEQ ID NO: 115)
GGTGTCGAGAAGAGGATGACAATATGCTAAATGTTGTTCTCGTCTCCTCGACACC
NNNNNNNCNNNNNNNNNN
v13:
(SEQ ID NO: 116)
GGTGTCGAGAAGAGGAGAACAATAGGCTAAACGTTGTTCTCGTCTCCTCGACACC
NNNNNNNCNNNNNNNNNN
dCas9Cj-NES-Linker-cdADAR2(E488Q)
(SEQ ID NO: 117)
MARILAFAIGISSIGWAFSENDELKDCGVRIFTKVENPKTGESLALPRRLARSARKR
LARRKARLNHLKIILIANEFKLNYEDYQSFDESLAKAYKGSLISPYELRFRALNELLS
KQDFARVILHIAKRRGYDDIKNSDDKEKGAILKAIKQNEEKLANYQSVGEYLYKEY
FQKFKENSKEFTNVRNKKESYERCIAQSFLKDELKLIFKKQREFGFSFSKKFEEEVL
SVAFYKRALKDFSHLVGNCSFFTDEKRAPKNSPLAFMFVALTRIINLLNNLKNTEGI
LYTKDDLNALLNEVLKNGTLTYKQTKKLLGLSDDYEFKGEKGTYFIEFKKYKEFI
KALGEHNLSQDDLNEIAKDITLIKDEIKLKKALAKYDLNQNQIDSLSKLEFKDHLNI
SFKALKLVTPLMLEGKKYDEACNELNLKVAINEDKKDFLPAFNETYYKDEVTNPV
VLRAIKEYRKVLNALLKKYGKVHKINIELAREVGKNHSQRAKIEKEQNENYKAKK
DAELECEKLGLKINSKNILKLRLFKEQKEFCAYSGEKIKISDLQDEKMLEIDAIYPYS
RSFDDSYMNKVLVFTKQNQEKLNQTPFEAFGNDSAKWQKIEVLAKNLPTKKQKRI
LDKNYKDKEQKNFKDRNLNDTRYIARLVLNYTKDYLDFLPLSDDENTKLNDTQKG
SKVHVEAKSGMLTSALRHTWGFSAKDRNNHLHHAIDAVIIAYANNSIVKAFSDFKK
EQESNSAELYAKKISELDYKNKRKFFEPFSGFRQKVLDKIDEIFVSKPERKKPSGAL
HEETFRKEEEFYQSYGGKEGVLKALELGKIRKVNGKIVKNGDMFRVDIFKHKKTN
KFYAVPIYTMDFALKVLPNKAVARSKKGEIKDWILMDENYEFCFSLYKDSLILIQT
KDMQEPEFVYYNAFTSSTVSLIVSKIIDNKFETLSKNQKILFKNANEKEVIAKSIGIQ
NLKVFEKYIVSALGEVTKAEFRQREDFKKSGLPPLERLTLGSGGGGSQLHLPQVLAD
AVSRLVLGKFGDLTDNFSSPHARRKVLAGVVMTTGTDVKDAKVISVSTGTKCINGEYM
SDRGLALNDCHAEIISRRSLLRFLYTQLELYLNNKDDQKRSIFQKSERGGFRLKENVQFH
LYISTSPCGDARIFSPHEPILEEPADRHPNRKARGQLRTKIESGQGTIPVRSNASIQTWDGV
LQGERLLTMSCSDKIARWNVVGIQGSLLSIFVEPIYFSSIILGSLYHGDHLSRAMYQRISNI
EDLPPLYTLNKPLLSGISNAEARQPGKAPNFSVNWTVGDSAIEVINATTGKDELGRASRL
CKHALYCRWMRVHGKVPSHLLRSKITKPNVYHESKLAAKEYQAAKARLFTAFIKAGLG
AWVEKPTEQDQFSLT
Single and dual ADAR2 recruiting domain:
Single:
(SEQ ID NO: 118)
GTGGAATAGTATAACAATATGCTAAATGTTGTTATAGTATCCCACACAAACCGA
GCGGTGTCTGT
Dual 1:
(SEQ ID NO: 119)
GTGGAATAGTATAACAATATGCTAAATGTTGTTATAGTATCCCACCAAACCGAG
CGGTGTCTGTGGTGGAATAGTATAACAATATGCTAAATGTTGTTATAGTATCCCAC
Dual 2:
(SEQ ID NO: 120)
GTGGAATAGTATAACAATATGCTAAATGTTGTTATAGTATCCCACTACAAACCG
AGCGGTGTCTGGTGGAATAGTATAACAATATGCTAAATGTTGTTATAGTATCCCAC
Dual 3:
(SEQ ID NO: 121)
GTGGAATAGTATAACAATATGCTAAATGTTGTTATAGTATCCCACTTTACAAACC
GAGCGGTGTCGTGGAATAGTATAACAATATGCTAAATGTTGTTATAGTATCCCAC
Dual 4:
(SEQ ID NO: 122)
GTGGAATAGTATAACAATATGCTAAATGTTGTTATAGTATCCCACGTTTTACAAA
CCGAGCGGTGGTGGAATAGTATAACAATATGCTAAATGTTGTTATAGTATCCCAC
Dual 5:
(SEQ ID NO: 123)
GTGGAATAGTATAACAATATGCTAAATGTTGTTATAGTATCCCACAAGTTTTACA
AACCGAGCGGGTGGAATAGTATAACAATATGCTAAATGTTGTTATAGTATCCCAC

### BRIEF DESCRIPTION OF THE DRAWINGS

FIG. 1 is a schematic of the vector constructs developed for the delivery of the modified endogenous or orthogonal tRNA.

FIG. 2A-B show suppression efficiencies of the tRNA constructs: (FIG. 2A) Relative efficiencies of the suppressor tRNAs derived from arginine, serine and leucine towards the amber, ochre and opal stop codons; Representative images showing the restoration of GFP expression in the presence of the Ser tRNAAmber (FIG. 2B) Comparison of the suppression efficiencies of the single or dual pyrrolysyl tRNAs towards amber, ochre and opal stop codons in the presence of 2 mM UAA; Representative images showing the relative GFP restoration using single and dual pyrrolysyl tRNAAmber in the presence of 2 mM UAA.

FIG. 3 shows the GFP reporter results for dystrophin with various tRNA and amino acids.

FIG. 4 shows the results of the dystrophin restoration experiments performed in mdx mice.

FIG. 5 shows sequences used to generate the ADAR2 constructs (SEQ ID NOS 164-166, respectively, in order of appearance).

FIG. 6 shows non-limiting examples of RNA level point mutations to a codon that can be made by ADAR2.

FIG. 7 shows exemplary schematics of constructs that may be used in an ADAR2 based RNA editing system.

FIG. 8 shows the results of optimization of the length of adRNA and distance of the edit from the ADAR2 recruiting domain. The first number in the shorthand for each category on the Y-axis is the length of adRNA and the second number (following the dash) is the distance of edit from ADAR2 recruiting domain. 20-6 with ADAR2 recruiting region v2 gave us the best results.

FIG. 9 shows in vitro restoration of GFP expression using the editing systems described herein.

FIG. 10 shows the results of optimization of hairpins with mismatches (SEQ ID NOS 167-172, respectively, in order of appearance). The first number in the shorthand for each category on the Y-axis is the number of mismatches and the second number is the number of bases it is from the target. For example, 13 is 1 mismatch, 3 bases away from the target.

FIG. 11 shows the results of varying lengths of toe hold, guide RNA sequences with no mismatches to the target.

FIG. 12A-C show results of (FIG. 12A) immunostaining, (FIG. 12B) Western blot, and (FIG. 12C) in vitro OTC mRNA editing assays (SEQ ID NOS 173-174, respectively, in order of appearance).

FIG. 13 is a Western blot that shows the restoration of dystrophin expression using suppressor tRNA, in comparison with the Cas9 based approaches.

FIG. 14 shows normalized dystrophin mRNA levels.

FIG. 15 shows results of immunostaining.

FIG. 16A-D shows in vitro suppression and editing of stop codons in GFP reporter mRNA: (FIG. 16A) Activity of arginine, serine and leucine suppressor tRNAs targeting amber, ochre and opal stop codons (n=3 independent replicates). (FIG. 16B) Orthogonal tRNA/aaRS (MbPylRS) based suppression of amber, ochre and opal stop codons in the presence of one or two copies of the pyrrolysyl-tRNA delivered via an AAV vector and in the presence of 1 mM NE-Boc-L-Lysine (n=3 independent replicates) (p-values 0.022, 0.002, 0.027 respectively). (FIG. 16C) ADAR2 based RNA editing efficiencies of amber and ochre stop codons, in one-step, two-steps, or in combination with suppressor tRNAs (n=3 independent replicates). (FIG. 16D) ADAR2 based RNA editing efficiencies of amber and ochre stop codons in the presence of one or two copies of the adRNA, delivered via an AAV vector (n=3 or 6 independent replicates) (p-values 0.0003, 0.0001, 0.0015 respectively).

FIG. 17A-E shows in vivo RNA targeting in mouse models of human disease: (FIG. 17A) Schematic of the DNA and RNA targeting approaches to restore dystrophin expression in mdx mice: (i) a dual gRNA-CRISPR based approach leading to in frame excision of exon 23; (ii) tRNA suppression of the ochre codon; and (iii) ADAR2 based editing of the ochre codon. (FIG. 17B) Immunofluorescence staining for dystrophin and nNOS in controls and treated samples (scale bar: 250 μm). (FIG. 17C) In vivo TAA->TGG/TAG/TGA RNA editing efficiencies in corresponding treated adult mdx mice (n=3 or 4 mice). (FIG. 17D) Schematic of the OTC locus in spf^{ash} mice which have a G->A point mutation at a donor splice site or missense in the last nucleotide of exon 4, and approach for correction of mutant OTC mRNA via ADAR2 mediated RNA editing (FIG. 17E) In vivo A->G RNA editing efficiencies in corresponding treated adult spf^{ash} mice (n=3 or 4 mice).

FIG. 18A-B show in vitro tRNA suppression evaluation and optimization: (FIG. 18A) Specificity of modified serine suppressor tRNAs for ochre and opal stop codons (n=3 independent replicates). (FIG. 18B) Ochre stop codon suppression efficiency utilizing three different aaRS: MbPylRS, MmPylRS and AcKRS, and two or four copies of the pyrroysyl-tRNA, or serine suppressor tRNA, all delivered using an AAV vector. MbPylRS, MmPylRS: 1 mM NE-Boc-L-Lysine; AcKRS: 1 or 10 mM Na-Acetyl-L-Lysine (n=3independent replicates).

FIG. 19A-C shows in vitro ADAR2 mediated site-specific RNA editing evaluation and optimization: (FIG. 19A) GFP expression is restored when adRNA/radRNA has two mismatches corresponding to the two adenosines in the ochre stop codon. Presence of a single mismatch results in the formation of an amber or opal stop codon (n=3independent replicates) (SEQ ID NOS 175-179, respectively, in order of appearance). (FIG. 19B) Panel of adRNA designs used (SEQ ID NOS 180-181, respectively, in order of appearance). (FIG. 19C) Optimization of adRNA antisense region using adRNA design 1: length and distance from the ADAR2 recruiting region were systematically varied, and editing efficiency calculated as a ratio of Sanger peak heights G/(A+G) (n=3 independent replicates) (SEQ ID NOS 182-206, respectively, in order of appearance).

FIG. 20A-C shows in vivo targeting of dystrophin mRNA via suppressor tRNAs: (FIG. 20A) Progressively increasing restoration of dystrophin expression over time in mdx mice treated with AAV8-dual-serine-ochre-tRNA. (FIG. 20B) UAA inducible nNOS localization in mdx mice treated with AAV8-dual-pyrrolysine-ochre-tRNA-MbPylRS. (FIG. 20C) Western blot for dystrophin shows partial recovery of dystrophin expression in the mdx mice treated with a serine tRNA ochre, the pyrrolysyl-tRNA ochre and administered with the UAA, as well as in Cas9/gRNAs treated samples.

FIG. 21A-D show in vitro and in vivo editing of dystrophin and OTC mRNA: (FIG. 21A) Representative Sanger sequencing plot showing 12.7% editing of the ochre stop codon (TAA->TGG) in a fragment of the mdx dystrophin mRNA expressed in HEK 293T cells (quantified using NGS) (SEQ ID NOS 207-208, respectively, in order of appearance). (FIG. 21B) Representative example of in vivo RNA editing analyses of treated mdx mouse (quantified using NGS) (SEQ ID NOS 209-216, respectively, in order of appearance). (FIG. 21C) Representative Sanger sequencing plot showing 29.7% correction of the point mutation in a fragment of the spf^{ash} OTC mRNA expressed in HEK 293T cells (quantified using NGS) (SEQ ID NOS 217-218, respectively, in order of appearance). (FIG. 21D) Representative example of in vivo RNA editing analyses of treated spf^{ash} mouse (quantified using NGS) (SEQ ID NOS 219-226, respectively, in order of appearance).

FIG. 22A-B show in vitro editing efficiency of ADAR2-E488Q. ADAR2-E488Q enables higher efficiency than the ADAR2 in the in vitro editing of: (FIG. 22A) a fragment of spf^{ash} OTC mRNA expressed in HEK293T cells (n=3 independent replicates) (p-value 0.037), and (FIG. 22B) a fragment of mdx dystrophin mRNA expressed in HEK293T cells (n=3 independent replicates) (p-values 0.048, 0.012 respectively). Efficiency was calculated as a ratio of Sanger peak heights G/(A+G).

FIG. 23A-D show schematics of (FIG. 23A) MCP or N22 fusions with ADAR1 or ADAR2, (FIG. 23B) recruitment of APOBEC by adRNA, (FIG. 23C) a more general adRNA architecture, and (FIG. 23D) the structure of the v2 adRNA scaffold after folding (SEQ ID NO: 227).

FIG. 24A-B show schematics of optional embodiments in which (FIG. 24A) endogenous ADAR2 can be used in the methods disclosed herein in tissues with high endogenous ADAR2, e.g., brain, lung, and spleen and (FIG. 24B) ADAR1 and/or ADAR2 levels can be increased in tissues with low levels of endogenous ADAR1 and ADAR2. Clockwise from the left, (1) delivery of adRNA and ADAR2 would result in high levels of RNA editing, (2) delivery of adRNA alone is likely to bring about little or no editing due to the low levels of endogenous ADAR1 and ADAR2, (3) treatment of cells with IFNs will lead to an increase in the ADAR1 (p150) levels but is unlikely to bring about any editing of the RNA target in the absence of the adRNA; (4) treatment of cells with IFNs with the addition of adRNA will lead to elevated levels of ADAR1 (p150) and in the presence of adRNA, is likely to lead to high levels of target RNA editing, (5) treatment of cells with IFNs with the addition of adRNA and ADAR2 will lead to elevated levels of ADAR1 expression, and high levels of RNA editing.

FIG. 25 shows the rate of UAA to UAG conversion. The UAA is converted to UAG via ADAR2 based editing and addition of suppressor tRNA targeting the UAG stop codon led to partial restoration of GFP expression

FIG. 26 shows the results of in vivo RNA editing in the mdx mouse model of muscular dystrophy.

FIG. 27 shows the resulting edited sequences resulting from use of the promiscuous C-terminal ADAR2 (SEQ ID NOS 228-264, respectively, in order of appearance).

FIG. 28 shows editing efficiency of the stabilized scaffolds (SEQ ID NOS 104-113, respectively, in order of appearance).

FIG. 29 shows the fraction of edited mRNA with single versus dual ADAR2 recruiting domains and the corresponding sequences (SEQ ID NOS 118-123, respectively, in order of appearance).

FIG. 30 shows the fraction of edited mRNA with various MCP-ADAR scaffolds (SEQ ID NOS 265-269, respectively, in order of appearance).

FIG. 31 shows alternative splice variants of OTC and is taken from Hodges, P. E. & Rosenberg, L. E. The spfash mouse: a missense mutation in the ornithine transcarbamylase gene also causes aberrant mRNA splicing. Proc. Natl. Acad. Sci. U.S.A. 86, 4142-4146 (1989) (SEQ ID NOS 270-275, respectively, in order of appearance).

### DETAILED DESCRIPTION

Unless defined otherwise, all technical and scientific terms used herein have the same meanings as commonly understood by one of ordinary skill in the art to which this invention belongs. All nucleotide sequences provided herein are presented in the 5′ to 3′ direction. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins cds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The terminology used in the description herein is for the purpose of describing particular embodiments only and is not intended to be limiting of the invention. All publications, patent applications, patents and other references mentioned herein are incorporated by reference in their entirety.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used in the specification and claims, the singular form “a”, “an” and “the” include plural references unless the context clearly dictates otherwise. For example, the term “a polypeptide” includes a plurality of polypeptides, including mixtures thereof.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

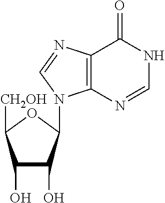

Inosine is read by the translation machinery as guanine (G). The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The terms “equivalent” or “biological equivalent” are used interchangeably when referring to a particular molecule, biological, or cellular material and intend those having minimal homology while still maintaining desired structure or functionality.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “functional” may be used to modify any molecule, biological, or cellular material to intend that it accomplishes a particular, specified effect.

As used herein, the terms “treating,” “treatment” and the like are used herein to mean obtaining a desired pharmacologic and/or physiologic effect. The effect may be prophylactic in terms of completely or partially preventing a disease, disorder, or condition or sign or symptom thereof, and/or may be therapeutic in terms of a partial or complete cure for a disorder and/or adverse effect attributable to the disorder.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The term “Cas9” refers to a CRISPR associated endonuclease referred to by this name (for example, UniProtKB G3ECR1 (CAS9_STRTR)) as well as dead Cas9 or dCas9, which lacks endonuclease activity (e.g., with mutations in both the RuvC and HNH domain). The term “Cas9” may further refer to equivalents of the referenced Cas9 having at least about 60%, 65%, 70%, 75%, 80%, 85%, 90%, 95%, or 99% identity thereto, including but not limited to other large Cas9 proteins. In some embodiments, the Cas9 is derived from Campylobacter jejuni or another Cas9 orthologue 1000 amino acids or less in length.

The term “vector” refers to a polynucleotide (usually DNA) used to artificially carry foreign genetic material to another cell where it can be replicated or expressed. Non-limiting exemplary vectors include plasmids, viral vectors, cosmids, and artificial chromosomes. Such vectors may be derived from a variety of sources, including bacterial and viral sources. A non-limiting exemplary viral source for a plasmid is adeno-associated virus.

As used herein, the term “recombinant expression system” refers to a genetic construct or constructs for the expression of certain genetic material formed by recombination; the term “construct” in this regard is interchangeable with the term “vector” as defined herein.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful for the purposes disclosed herein include any of the 11 serotypes, e.g., AAV2 and AAV8.

The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy—Tools and Potential Applications,” ISBN 978-953-51-1014-9, DOI: 10.5772/52534.

As used herein the term “restoring” in relation to expression of a protein refers to the ability to establish expression of full length protein where previously protein expression was truncated due to mutation.

The term “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site.

“Messenger RNA” or “mRNA” is a nucleic acid molecule that is transcribed from DNA and then processed to remove non-coding sections known as introns. The resulting mRNA is exported from the nucleus (or another locus where the DNA is present) and translated into a protein. The term “pre-mRNA” refers to the strand prior to processing to remove non-coding sections.

“Transfer ribonucleic acid” or “tRNA” is a nucleic acid molecule that helps translate mRNA to protein. tRNA have a distinctive folded structure, comprising three hairpin loops; one of these loops comprises a “stem” portion that encodes an anticodon. The anticodon recognizes the corresponding codon on the mRNA. Each tRNA is “charged with” an amino acid corresponding to the mRNA codon; this “charging” is accomplished by the enzyme tRNA synthetase. Upon tRNA recognition of the codon corresponding to its anticodon, the tRNA transfers the amino acid with which it is charged to the growing amino acid chain to form a polypeptide or protein. Endogenous tRNA can be charged by endogenous tRNA synthetase. Accordingly, endogenous tRNA are typically charged with canonical amino acids. Orthogonal tRNA, derived from an external source, require a corresponding orthogonal tRNA synthetase. Such orthogonal tRNAs may be charged with both canonical and non-canonical amino acids. In some embodiments, the amino acid with which the tRNA is charged may be detectably labeled to enable detection in vivo. Techniques for labeling are known in the art and include, but are not limited to, click chemistry wherein an azide/alkyne containing unnatural amino acid is added by the orthogonal tRNA/synthetase pair and, thus, can be detected using alkyne/azide comprising fluorophore or other such molecule.

The term “stop codon” intends a three nucleotide contiguous sequence within messenger RNA that signals a termination of translation. Non-limiting examples include in RNA, UAG, UAA, UGA and in DNA TAG, TAA or TGA. Unless otherwise noted, the term also includes nonsense mutations within DNA or RNA that introduce a premature stop codon, causing any resulting protein to be abnormally shortened. tRNA that correspond to the various stop codons are known by specific names: amber (UAG), ochre (UAA), and opal (UGA).

“Canonical amino acids” refer to those 20 amino acids found naturally in the human body shown in the table below with each of their three letter abbreviations, one letter abbreviations, structures, and corresponding codons:

non-polar, aliphatic residues

| | | | | |
|---|---|---|---|---|
| Glycine | Gly | G | 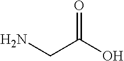 | GGU GGC GGA GGG |
| Alanine | Ala | A | 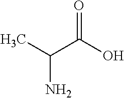 | GCU GCC GCA GCG |
| Valine | Val | V | 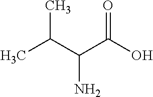 | GUU GUC GUA GUG |
| Leucine | Leu | L | 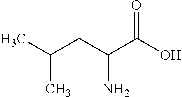 | UUA UUG CUU CUC CUA CUG |
| Isoleucine | Ile | I | 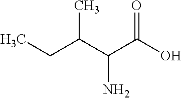 | AUU AUC AUA |
| Proline | Pro | P | 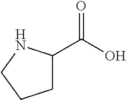 | CCU CCC CCA CCG |

aromatic residues

| | | | | |
|---|---|---|---|---|
| Phenylalanine | Phe | F | 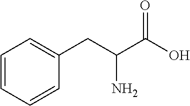 [SMILES: NC(Cc1ccccc1)C(=O)O] | UUU UUC |
| Tyrosine | Tyr | Y | 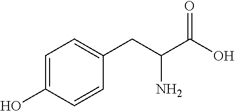 [SMILES: NC(Cc1ccc(O)cc1)C(=O)O] | UAU UAC |
| Tryptophan | Trp | W | 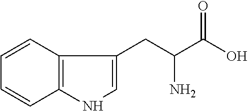 [SMILES: NC(Cc1c[nH]c2ccccc12)C(=O)O] | UGG |

polar, non-charged residues

| | | | | |
|---|---|---|---|---|
| Serine | Ser | S | 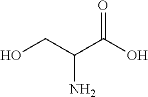 | UCU UCC UCA UCG AGU AGC |
| Threonine | Thr | T | 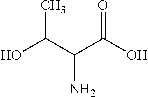 | ACU ACC ACA ACG |
| Cysteine | Cys | C | 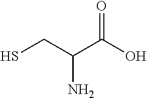 | UGU UGC |
| Methionine | Met | M | 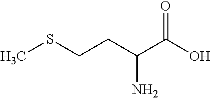 | AUG |
| Asparagine | Asn | N | 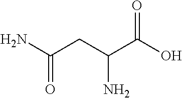 | AAU AAC |
| Glutamine | Gln | Q | 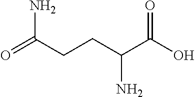 | CAA CAG |

positively charged residues

| | | | | |
|---|---|---|---|---|
| Lysine | Lys | K | 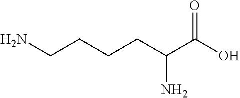 | AAA AAG |
| Arginine | Arg | R | 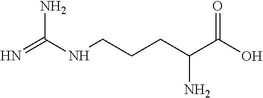 [SMILES: N=C(N)NCCCC(N)C(=O)O] | CGU CGC CGA CGG AGA AGG |
| Histidine | His | H | 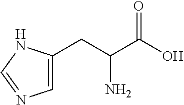 [SMILES: NC(Cc1cnc[nH]1)C(=O)O] | CAU CAC |

negatively charged residues

| | | | | |
|---|---|---|---|---|
| Aspartate | Asp | D | 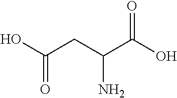 | GAU GAC |
| Glutamate | Glu | E | 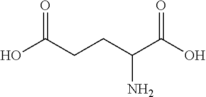 | GAA GAG |

The term “non-canonical amino acids” refers to those synthetic or otherwise modified amino acids that fall outside this group, typically generated by chemical synthesis or modification of canonical amino acids (e.g. amino acid analogs). The present disclosure employs proteinogenic non-canonical amino acids in some of the methods and vectors disclosed herein. A non-limiting exemplary non-canonical amino acid is pyrrolysine (Pyl or O), the chemical structure of which is provided below:

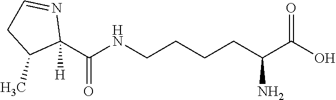 [SMILES: C[C@@H]1CC=N[C@H]1C(=O)NCCCC[C@H](N)C(=O)O]

Inosine (I) is another exemplary non-canonical amino acid, which is commonly found in tRNA and is essential for proper translation according to “wobble base pairing.” The structure of inosine is provided above.

The term “ADAR” as used herein refers to an adenosine deaminase that can convert adenosines (A) to inosines (I) in an RNA sequence. ADAR1 and ADAR2 are two exemplary species of ADAR that are involved in mRNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218;UniProtKB: P78563; and GeneCards: GC21P045073, as well as biological equivalents thereof. Further non-limited exemplary sequences of the catalytic domain are provided hereinabove. The forward and reverse RNA used to direct site-specific ADAR editing are known as “adRNA” and “radRNA,” respectively. The catalytic domains of ADAR1 and ADAR2 are comprised in the sequences provided herein below.

ADAR1 catalytic domain:
(SEQ ID NO: 124)
KAERMGFTEVTPVTGASLRRTMLLLSRSPEAQPKTLPLTGSTFHDQIAM
LSHRCFNTLTNSFQPSLLGRKILAAIIMKKDSEDMGVVVSLGTGNRCVK
GDSLSLKGETVNDCHAEIISRRGFIRFLYSELMKYNSQTAKDSIFEPAK
GGEKLQIKKTVSFHLYISTAPCGDGALFDKSCSDRAMESTESRHYPVFE
NPKQGKLRTKVENGEGTIPVESSDIVPTWDGIRLGERLRTMSCSDKILR
WNVLGLQGALLTHFLQPIYLKSVTLGYLFSQGHLTRAICCRVTRDGSAF
EDGLRHPFIVNHPKVGRVSIYDSKRQSGKTKETSVNWCLADGYDLEILD
GTRGTVDGPRNELSRVSKKNIFLLFKKLCSFRYRRDLLRLSYGEAKKAA
RDYETAKNYFKKGLKDMGYGNWISKPQEEKNFYLCPV
ADAR2 catalytic domain:
(SEQ ID NO: 125)
QLHLPQVLADAVSRLVLGKFGDLTDNFSSPHARRKVLAGVVMTTGTDVK
DAKVISVSTGTKCINGEYMSDRGLALNDCHAEIISRRSLLRFLYTQLEL
YLNNKDDQKRSIFQKSERGGFRLKENVQFHLYISTSPCGDARIFSPHEP
ILEEPADRHPNRKARGQLRTKIESGEGTIPVRSNASIQTWDGVLQGERL
LTMSCSDKIARWNVVGIQGSLLSIFVEPIYFSSIILGSLYHGDHLSRAM
YQRISNIEDLPPLYTLNKPLLSGISNAEARQPGKAPNFSVNWTVGDSAI
EVINATTGKDELGRASRLCKHALYCRWMRVHGKVPSHLLRSKITKPNVY
HESKLAAKEYQAAKARLFTAFIKAGLGAWVEKPTEQDQFSLT

The double stranded RNA binding domains (dsRBD) of an ADAR is comprised in the sequence provided herein below.

(SEQ ID NO: 126)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFN

It is appreciated that further mutations can be made to the sequence of the ADAR and/or its various domains. For example, Applicants have generated E488Q and E1008Q mutants of both ADAR1 and ADAR2, as well as a “promiscuous” variant of ADAR2—resulting from a C-terminal deletion. This “promiscuous” variant is known as such because it demonstrated promiscuity in edited reads with several As close to a target sequence showing an A to G conversion (verified across 2 different loci). The sequence of this variant is provided herein below.

(SEQ ID NO: 127)
MLRSFVQFPNASEAHLAMGRTLSVNTDFTSDQADFPDTLFNGFETPDKAE
PPFYVGSNGDDSFSSSGDLSLSASPVPASLAQPPLPVLPPFPPPSGKNPV
MILNELRPGLKYDFLSESGESHAKSFVMSVVVDGQFFEGSGRNKKLAKAR
AAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRG
LALNDCHAEIISRRSLLRFLYTQLELYLNNKDDQKRSIFQKSERGGFRLK
ENVQFHLYISTSPCGDARIFSPHEPILEEPADRHPNRKARGQLRTKIESG
EGTIPVRSNASIQTWDGVLQGERLLTMSCSDKIARWNVVGIQGSLLSIFV
EPIYFSSIILGSLYHGDHLSRAMYQRISNIEDLPPLYTLNKPLLSGISNA
EARQPGKAPNFSVNWTVGDSAIEVINATTGKDELGRASRLCKHALYCRWM
RVHGKVPSHLLRSKITKPNVYHESKLAAKEYQAAKARLFTAFIKAGLGAW
VEKPTEQDQFSLTP*

Not to be bound by theory, a C-terminal deletion in ADAR1 may produce the same or similar effect.

The term “deficiency” as used herein refers to lower than normal (physiologically acceptable) levels of a particular agent. In context of a protein, a deficiency refers to lower than normal levels of the full length protein.

The term “dystrophin” as used herein refers to the protein corresponding with that name and encoded by the gene Dmd; a non-limiting example of which is found under UniProt Reference Number P11532 (for humans) and P11531 (for mice).

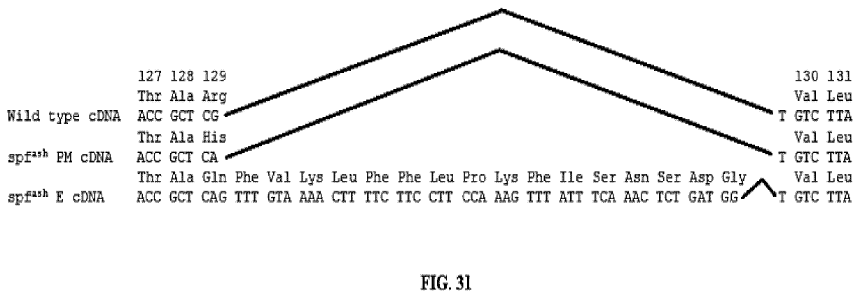

The term “ornithine transcarbamylase” or “OTC” as used herein refers to the protein corresponding with that name and encoded by the gene Otc; a non-limiting example of which is found under UniProt Reference Number P00480 (for humans) and P11725 (for mice). OTC deficiency is an X-linked genetic condition resulting in high concentrations of ammonia in blood. In some cases, OTC deficiency is caused by a G->A splice site mutation in the donor splice site of exon 4 that results in mis-splicing of the pre-mRNA. This mutation results in the formation of a protein that either is elongated or bears a point mutation. There is a 15-20 fold reduction in the OTC protein levels. The FIG. 31 (taken from Hodges, P. E. & Rosenberg, L. E. The spfash mouse: a missense mutation in the ornithine transcarbamylase gene also causes aberrant mRNA splicing. Proc. Natl. Acad. Sci. U.S.A. 86, 4142-4146 (1989)) shows the alternative forms produced. The sequences thereof are provided below:

OTC pre-mRNA (wild type):
(SEQ ID NO: 128)
.....CTCACAGACCGCTCGGTTTGTAAAACTTTTCTTC.....
OTC pre-mRNA mutant:
(SEQ ID NO: 129)
.....CTCACAGACCGCTCAGTTTGTAAAACTTTTCTTC.....
OTC mRNA (incorrectly spliced mutant):
(SEQ ID NO: 130)
.....CTCACAGACACCGCTCAGTTTGTAAAACTTTTCTTC.....
OTC mRNA (correctly spliced, mutant):
(SEQ ID NO: 131)
.....CTCACAGACACCGCTCATGTCTTATCTAGCATGACA.....
OTC mRNA (correctly spliced, wild type):
(SEQ ID NO: 132)
.....CTCACAGACACCGCTCGTGTCTTATCTAGCATGACA.....

As shown above, a correct splice variant may be produced when the mutation is present; however, such production results in a missense mutation, which also can contribute to OTC deficiency.

The terms “hairpin,” “hairpin loop,” “stem loop,” and/or “loop” used alone or in combination with “motif” is used in context of an oligonucleotide to refer to a structure formed in single stranded oligonucleotide when sequences within the single strand which are complementary when read in opposite directions base pair to form a region whose conformation resembles a hairpin or loop.

As used herein, the term “domain” refers to a particular region of a protein or polypeptide and is associated with a particular function. For example, “a domain which associates with an RNA hairpin motif” refers to the domain of a protein that binds one or more RNA hairpin. This binding may optionally be specific to a particular hairpin. For example, the M2 bacteriophage coat protein (MCP) is capable of specifically binding to particular stem-loop structures, including but not limited to the MS2 stem loop. See, e.g. Peabody, D.S., “The RNA binding site of bacteriophage MS2 coat protein.” EMBO J. 12(2):595-600 (1993); Corrigan and Chubb, “Biophysical Methods in Cell Biology” Methods in Cell Biology (2015). Similarly, λN22—referred to herein as “N22 peptide” is capable of specifically binding to particular stem-loop structures, including but not limited to BoxB stem loops. See, e.g., Cilley and Williamson, “Analysis of bacteriophage N protein and peptide binding to boxB RNA using polyacrylamide gel coelectrophoresis (PACE).” RNA 3(1):57-67 (1997). The sequences of both MCP and MS2 stem loop and N22 peptide and BoxB loop are provided hereinabove in context of fusion proteins with an ADAR (MCP, N22 peptide) and use in adRNA (MS2 stem loop, BoxB loop), respectively.

The term “APOBEC” as used herein refers to any protein that falls within the family of evolutionarily conserved cytidine deaminases involved in mRNA editing—catalyzing a C to U conversion—and equivalents thereof. In some aspects, the term APOBEC refers to any one of APOBEC1, APOBEC2, APOBEC3A, APOBEC3B, APOBEC3C, APOBEC3E, APOBEC3F, APOBEC3G, APOBEC3H, APOBEC4, or equivalents each thereof. Non-limiting exemplary sequences of fusion proteins comprising one or more APOBEC domains are provided herein both fused to an ADAR domain or fused to alternative domains to render them suitable for use in an RNA editing system. To this end, APOBECs can be considered an equivalent of ADAR—catalyzing editing albeit by a different conversion. Thus, not to be bound by theory, Applicants believe that all embodiments contemplated herein for use with an ADAR based editing system may be adapted for use in an APOBEC based RNA editing system.

As used herein, the term “interferon” refers to a group of signaling proteins known to be associated with the immune response. In context of this application, the interferons of interest are those that result in enhanced expression of an ADAR. The correlation between interferon α and ADAR1 is well known, and, thus, the present disclosure contemplates use of interferon α as a means of increasing endogenous ADAR1 expression. Commercial sources of isolated or recombinant interferon α include but are not limited to Sigma-Aldrich, R&D Systems, Abcam, and Thermo Fisher Scientific. Alternatively, interferon α may be produced using a known vector and given protein sequence, e.g. Q6QNB6 (human IFNA).

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### MODES OF CARRYING OUT THE DISCLOSURE

Point mutations underlie many genetic diseases. In this regard, while programmable DNA nucleases have been used to repair mutations, their use for gene therapy poses multiple challenges: one, efficiency of homologous recombination is typically low in cells; two, an active nuclease presents a risk of introducing permanent off-target mutations; and three, prevalent programmable nucleases typically comprise elements of non-human origin raising the potential of in vivo immunogenicity. In light of these, approaches to instead directly target RNA, and use of molecular machinery native to the host would be highly desirable. Towards this, Applicants have engineered and optimized two complementary approaches, referred together hereon as tRiAD, based on the use of tRNAs in codon suppression and adenosine deaminases in RNA editing. Specifically, by delivering modified endogenous tRNAs or the RNA editing enzyme ADAR and an associated guiding RNA (adRNA) via adeno-associated viruses, Applicants enabled premature stop codon read-through and correction in the mdx mouse model of muscular dystrophy that harbors a nonsense mutation in the dystrophin gene. Additionally, Applicants engineered ADAR2 mediated correction of a point mutation in liver RNA of the spf^{ash} mouse model of ornithine transcarbamylase (OTC) deficiency. Taken together, the results disclosed herein establish the use of suppressor tRNAs and ADAR2 for in vivo RNA targeting, and this integrated tRiAD approach is robust, genomically scarless, and potentially non-immunogenic, as it utilizes effector RNAs and human proteins.

Aspects of the disclosure relate to a tRNA based protein editing system optionally alone or in combination with an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits a point mutation in an RNA sequence encoding a gene.

The tRNA based protein editing system may comprise endogenous modified tRNA and/or orthogonal tRNA in order to prevent off target editing of proteins. In this regard, systems for the control of these tRNA are disclosed herein below.

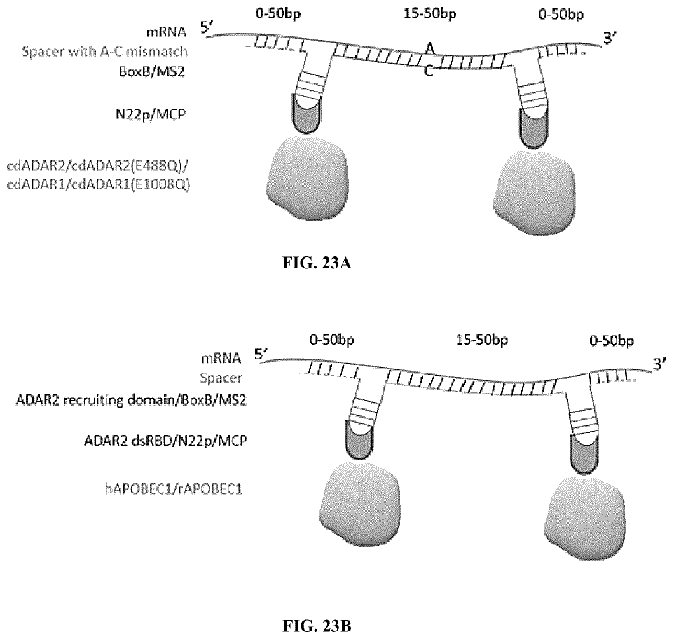

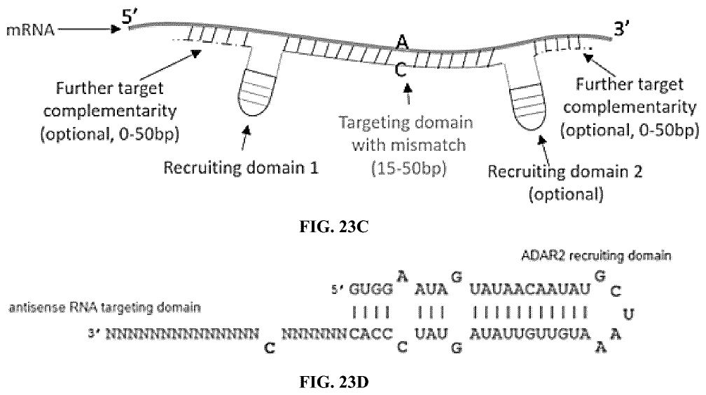

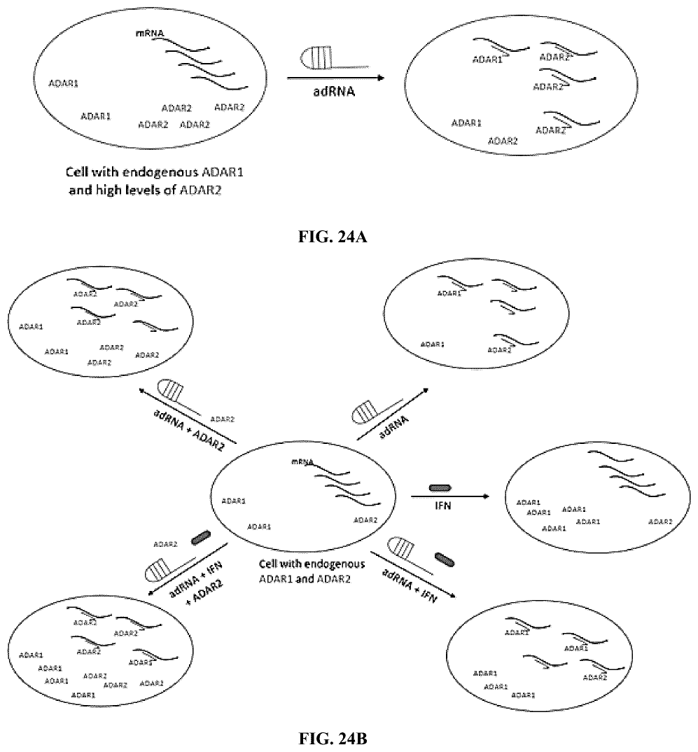

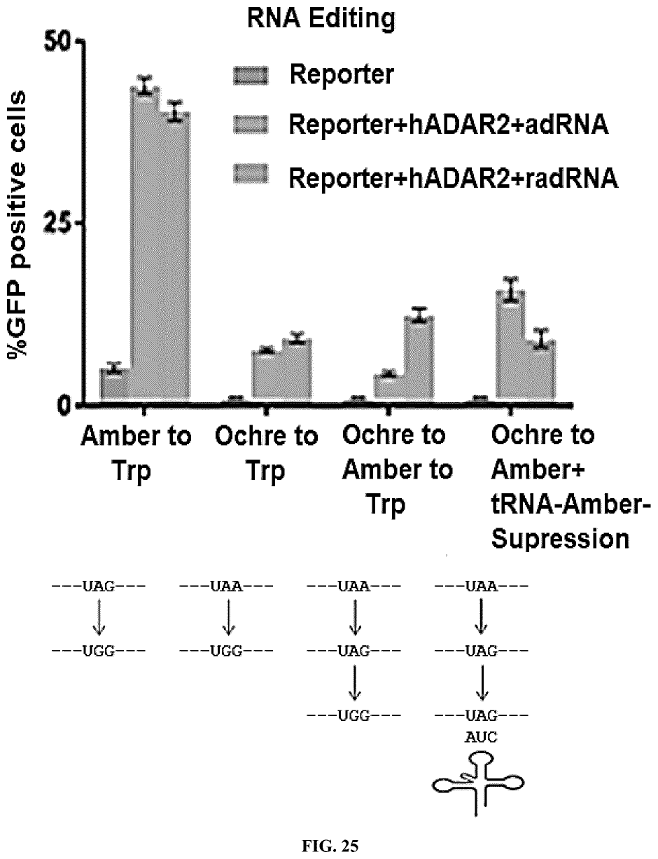

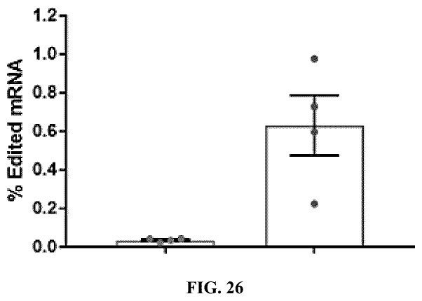

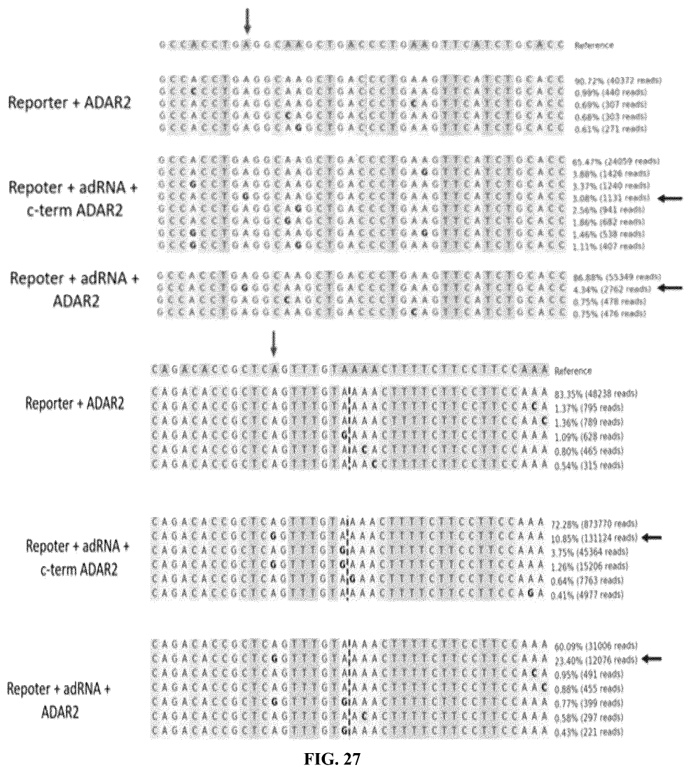

The adRNA architecture for use in the ADAR based RNA editing system is relatively simple, comprising a RNA targeting domain, complementary to the target and, optionally, one or two recruiting domains (also referred to as aptamers) that recruit RNA binding domains of various proteins. The optional recruiting domains are positioned at the 5′ and/or 3′ ends of the RNA targeting domain. A schematic of adRNA bound to its mRNA target is provided in FIG. 23C. In some embodiments, the adRNA features an A-C mismatch, which prompts editing function of the ADAR. A similar framework can be used to target pre-mRNA, prior to intron processing by adapting the scaffold to target the pre-mRNA present in the nucleus. This approach is taken in the non-limiting exemplary methods involving OTC deficiency—involving a splice site mutation, whereas an mRNA editing approach is taken in the non-limiting exemplary methods involving dystrophin deficiency—involving a nonsense mutation.

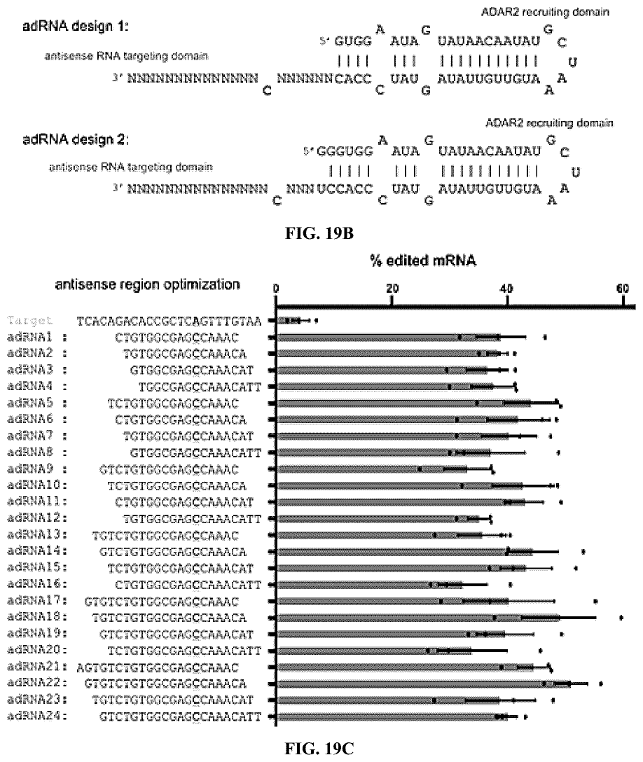

Applicants tested a series of scaffolds, shown in FIG. 19C, to recruit RNA binding domains of the ADARs. The sequences provided in the figure represent the recruiting domain and the italicized Ns represent the nucleotides complimentary to the target. The C is the mismatch that prompts the editing function. Sequences of varying length and mismatch position may be tested to determine the best adRNA for the desired target. For example, residues in the recruiting domain of the adRNAs generated by Applicants were modified as follows (5′-3′):

v1:
(SEQ ID NO: 104)
GGGTGGAATAGTATAACAATATGCTAAATGTTGTTATAGTATCCCACCT NNNCNNNNNNNNNNNNNNN
v2:
(SEQ ID NO: 105)
GTGGAATAGTATAACAATATGCTAAATGTTGTTATAGTATCCCAC NNNNNNCNNNNNNNNNNNNN
v3:
(SEQ ID NO: 106)
GTGGAAGAGGAGAACAATATGCTAAATGTTGTTCTCGTCTCCCAC NNNNNNCNNNNNNNNNNNNNN
v4:
(SEQ ID NO: 107)
GGGTGGAAGAGGAGAACAATATGCTAAATGTTGTTCTCGTCTCCCACCT NNNCNNNNNNNNNNNNNNN
v5:
(SEQ ID NO: 108)
GGTGAAGAGGAGAACAATATGCTAAATGTTGTTCTCGTCTCCACC NNNNNNCNNNNNNNNNNNNNN
v6:
(SEQ ID NO: 109)
GGTGAAGAGGAGAACAATATGCTAAATGTTGTTCTCGTCTCCACC NNNNNNNCNNNNNNNNNNNNN
v7:
(SEQ ID NO: 110)
GTGGAAGAGGAGAACAATAGGCTAAACGTTGTTCTCGTCTCCCAC NNNNNNCNNNNNNNNNNNNNN
v8:
(SEQ ID NO: 111)
GGGTGGAAGAGGAGAACAATAGGCTAAACGTTGTTCTCGTCTCCCACCT NNNCNNNNNNNNNNNNNNN
v9:
(SEQ ID NO: 112)
GGTGAAGAGGAGAACAATAGCTAAACGTTGTTCTCGTCTCCACC NNNNNNCNNNNNNNNNNNNNN
v10:
(SEQ ID NO: 113)
GGTGAAGAGGAGAACAATAGCTAAACGTTGTTCTCGTCTCCACC NNNNNNNCNNNNNNNNNNNN
v11:
(SEQ ID NO: 114)
GGTGTCGAGAATAGTATAACCAATATGCTAAATGTTGTTATAGTATCCTCGACACC NNNNNNNCNNNNNNNNNN
v12:
(SEQ ID NO: 115)
GGTGTCGAGAAGAGGATGACAATATGCTAAATGTTGTTCTCGTCTCCTCGACACC NNNNNNNCNNNNNNNNNN
v13:
(SEQ ID NO: 116)
GGTGTCGAGAAGAGGAGAACAATAGGCTAAACGTTGTTCTCGTCTCCTCGACACC NNNNNNNCNNNNNNNNNN

The structure of V2 after folding is provided as FIG. 23D. And the corresponding radRNAs were generated as follows:

(SEQ ID NO: 133)
NNNNNNNNNNNNNNNCNNN TCCACCCTATGATATTGTTGTAAATCGTATAACAATATGATAAGGTGGG
(SEQ ID NO: 134)
NNNNNNNNNNNNNCNNNNNN CACCCTATGATATTGTTGTAAATCGTATAACAATATGATAAGGTG
(SEQ ID NO: 135)
NNNNNNNNNNNNNNCNNNNNN CACCCTCTGCTCTTGTTGTAAATCGTATAACAAGAGGAGAAGGTG
(SEQ ID NO: 136)
NNNNNNNNNNNNNNNCNNN TCCACCCTCTGCTCTTGTTGTAAATCGTATAACAAGAGGAGAAGGTGGG
(SEQ ID NO: 137)
NNNNNNNNNNNNNNCNNNNNN CCACCTCTGCTCTTGTTGTAAATCGTATAACAAGAGGAGAAGTGG
(SEQ ID NO: 138)
NNNNNNNNNNNNNCNNNNNNN CCACCTCTGCTCTTGTTGTAAATCGTATAACAAGAGGAGAAGTGG
(SEQ ID NO: 139)
NNNNNNNNNNNNNNCNNNNNN CACCCTCTGCTCTTGTTGCAAATCGGATAACAAGAGGAGAAGGTG
(SEQ ID NO: 140)
NNNNNNNNNNNNNNNCNNN TCCACCCTCTGCTCTTGTTGCAAATCGGATAACAAGAGGAGAAGGTGGG
(SEQ ID NO: 141)
NNNNNNNNNNNNNNCNNNNNN CCACCTCTGCTCTTGTTGCAAATCGGATAACAAGAGGAGAAGTGG
(SEQ ID NO: 142)
NNNNNNNNNNNNNCNNNNNNN CCACCTCTGCTCTTGTTGCAAATCGGATAACAAGAGGAGAAGTGG
(SEQ ID NO: 143)
NNNNNNNNNNCNNNNNNN CCACAGCTCCTCTGCTCTTGTTGCAAATCGGATAACAAGAGGAGAAGAGCTGTGG
(SEQ ID NO: 144)
NNNNNNNNNNCNNNNNNN CCACAGCTCCTCTGCTCTTGTTGTAAATCGTATAACAAGAGGAGAAGAGCTGTGG
(SEQ ID NO: 145)
NNNNNNNNNNCNNNNNNN CCACAGCTCCTATGATATTGTTGTAAATCGTATAACAATATGATAAGAGCTGTGG

A schematic of the resulting adRNA and radRNA pairings to the target mRNA is shown in FIG. 16C.

An alternative scaffold framework was also applied by Applicants using two ADAR recruiting domains (black font) on either side of the targeting domain while varying the position of the C mismatch in the targeting domain (italicized Ns).

(SEQ ID NO: 146)
TGGAATAGTATAACAATATGCTAAATGTTGTTATAGTATCCCAC
NNNNNNNNNNNNNNNNNNNNGTGGAATAGTATAACAATATGC
TAAATGTTGTTATAGTATCCCAC

These non-limiting exemplary scaffolds provide a template for the engineering of adRNA and radRNA for particular targets and may be optimized based on comparative efficacy studies carried out according to the exemplary methods disclosed herein.

In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the adRNA comprises one or more RNA hairpin motifs. In some embodiments, the one or more RNA hairpin motifs are selected from the group of an MS2 stem loop and a BoxB loop.

Not to be bound by theory, Applicants believe the double stranded RNA binding motif from ADARs may bind to several double stranded RNA sequences and could thus have possible off target effects. To avoid such effects, Applicants contemplate the use of exogenous protein domains to recognize RNA hairpin motifs in the adRNA. Both ADAR1 and ADAR2 consist of RNA binding domains and a catalytic domain that catalyzes the conversion of adenosine to inosine. The catalytic domain can be uncoupled from the RNA binding domain. Our aim is to achieve high editing efficiency of the targeted adenosine while reducing off target effects and thus are exploring alternative RNA binding domains. Applicants have fused the catalytic domain of the ADAR1 or ADAR2 to different RNA binding domains such as the MCP, N22 or a dead CjCas9 (or other RNA targeting CRISPRs such as from SaCas9, CRISPR-Cas13 etc.). Upon the addition of appropriate guide RNAs (adRNAs), the fusion proteins are recruited to the target, further catalyzing an adenosine to inosine change. The dead CjCas9 (and other CRISPRs by extension) in this case basically serves as a RNA binding domain that can in turn be tethered to effectors.

The domains are fused to the ADAR catalytic domain to generate ADAR specifically targeting the particular adRNA comprising the RNA hairpin motifs. For example, Applicants have used a MS2 bacteriophage coat protein (MCP) fused to either the catalytic domain of ADAR1 or ADAR2 and their respective mutants E488Q and E1008Q, while using a MS2 stem loop on the RNA to recruit the fusion protein (FIG. 23A). Analogous to this system, Applicants have also utilized a N22 peptide fused to the catalytic domains of ADAR1 or ADAR2 (and their mutants) while making use of a boxB aptamer to recruit the fusion protein. Thus, one or two copies of ADAR may be recruited based on the addition of single or dual hairpins (MS2/BoxB loops) (FIG. 23A). PP7 hairpins are also contemplated for use in the same manner.

A non-limiting framework sequence for the recruitment of MCP-based fusion proteins, where the C mismatch may be varied within the targeting domain, is provided herein below (with the lower case letters representing those linkers that help stabilize the underlined hairpins):

(SEQ ID NO: 98)
NNNNNNNNNNNNNNNNNNNNggccAACATGAGGATCACCCATGTCTGCAGg
gcc
Two recruiting domains (underlined):
(SEQ ID NO: 99)
aACATGAGGATCACCCATGTcNNNNNNNNNNNNNNNNNNNNaACATGAGGA
TCACCCATGTc

An analogous non-limiting framework sequence is provided for the N22-based fusion proteins:

(SEQ ID NO: 100)
NNNNNNNNNNNNNNNNNNNNgggccctgaagaagggccc
Two recruiting domains (underlined):
(SEQ ID NO: 101)
ggGCCCTGAAGAAGGGCccNNNNNNNNNNNNNNNNNNNNggGCCCTGAAGA
AGGGCcc

Another approach is to recruitment domains in the adRNA associated with Cas9 and couple a dead Cas9 to the ADAR catalytic domain, thus, rendering the same effect of specific recruitment. A non-limiting framework sequence for the recruitment is provided for Cas9-based fusion proteins:

Psp dCas13a recruitment
(mismatch position can be varied)
(SEQ ID NO: 147)
CAACATTATCGGGGAGTTTTGACCTCCAAGGTGTTGNNNNNNNNNNNNNNN
NNNNNNNNNNNNNNNNNNNNNNNNNNNNNNNNNNN
Cj dCas9 recruitment
(mismatch position can be varied)
(SEQ ID NO: 148)
NNNNNNNNNNNNNNNNNNNNNNgttttagtccctgaaaagggactaaaata
aagagtttgcgggactctgcggggttacaatcccctaaaaccgcttttttt

APOBECs also have RNA editing function (FIG. 23B). Thus, they may be used in the alternative or in addition to the ADAR based editing system. For example, Applicants have created MCP/N22 peptide fusions with APOBECs to engineer targeted C->T RNA editing. In addition, Applicants have fused the double stranded RNA binding domains (dsRBD) of the ADAR2 with APOBECs as a result of which the APOBECs can be recruited by the adRNA.

The addition of Nuclear Localization Signals (NLS) to the fusion protein could help target nuclear RNA (i.e. pre-mRNA) while addition of Nuclear Export Signals (NES) to the fusion protein could help target cytoplasmic RNA in any of the embodiments disclosed herein. This method is useful when editing for splice site mutations, which result in incorrect processing of introns in the pre-mRNA and, thus, results in incorrect mRNA for translation. OTC deficiency is example where targeting pre-mRNA with adRNA scaffolds can be useful, since the majority of aberrant OTC expression comes from the splice site mutation resulting in a truncated OTC protein. Further addition of RNA localization tags to the adRNA will enable targeting RNA in specific cellular compartments.

In further embodiments where the adRNA comprises one or more RNA hairpin motifs, the one or more RNA hairpin motifs are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA.

More generally, can be appreciated that the RNA targeting domains of adRNAs are designed such that they are complementary to the target mRNA while containing C mismatch at the position of the target adenosine. The recruiting domains of the adRNA are constant. BY way of non-limiting example:

Example target: OTC mRNA (mutation underlined)
(SEQ ID NO: 149)
5'-AAAGTCTCACAGACACCGCTCAGTTTGTAAAACTTTTCTTC-3'
adRNA v2 (targeting domain length 20bp,
mismatch position after 6 bases):
(SEQ ID NO: 149)
5'-AAAGTCTCACAGACACCGCTCAGTTTGTAAAACTTTTCTTC-3'
(SEQ ID NO: 150)
5'-GTGGAATAGTATAACAATATGCTAAATGTTGTTATAGTATC
CCACTGTGTGTGGCGAGCCAAACA-3'
adRNA v2 (targeting domain length 21bp, mismatch
position after 6 bases):
(SEQ ID NO: 149)
5'-AAAGTCTCACAGACACCGCTCAGTTTGTAAAACTTTTCTTC-3'
(SEQ ID NO: 151)
5'-GTGGAATAGTATAACAATATGCTAAATGTTGTTA
TAGTATCCCACGTGTCTGTGGCGAGCCAAACA-3'
radRNA v2 (targeting domain length 20bp, mismatch
position after 6 bases):
(SEQ ID NO: 149)
3'-CTTCTTTTCAAAATGTTTGACTCGCCACAGACACTCTGAAA-5'
(SEQ ID NO: 152)
5'-AAGTTTTACAAACCGAGCGGCACCCTATGATATTGTTGTAAAT
CGTATAACAATATGATAAGGTG-3'
adRNA dual (targeting domain length 20bp, mismatch
position after 5, 14 bases):
(SEQ ID NO: 149)
3'-CTTCTTTTCAAAATGTTTGACTCGCCACAGACACTCTGAAA-5'
(SEQ ID NO: 153)
5'-TGGAATAGTATAACAATATGCTAAATGTTGTTATAGTATCCCAC
CAAACCGAGCGGTGTCTGTGGTGGAATAGTATAACAATATGCTAAAT
GTTGTTATAGTATCCCAC-3'
adRNA MS2 (targeting domain length 20bp, mismatch
position after 14 bases)
(SEQ ID NO: 149)
3'-CTTCTTTTCAAAATGTTTGACTCGCCACAGACACTCTGAAA-5'
(SEQ ID NO: 154)
5'-CAAACCGAGCGGTGTCTGTGggccAACATGAGGATCACCCATGT
CTGCAGggcc-3'
adRNA MS2 dual (targeting domain length 20bp,
mismatch position after 5, 14 bases)
(SEQ ID NO: 149)
3'-CTTCTTTTCAAAATGTTTGACTCGCCACAGACACTCTGAAA-5'
(SEQ ID NO: 155)
5'-aACATGAGGATCACCCATGTcCCAAACCGAGCGGTGTCTGTGaA
CATGAGGATCACCCATGTc-3'
adRNA BoxB (targeting domain length 20bp, mismatch
position after 14 bases)
(SEQ ID NO: 149)
3'-CTTCTTTTCAAAATGTTTGACTCGCCACAGACACTCTGAAA-5'
(SEQ ID NO: 156)
5'-CAAACCGAGCGGTGTCTGTGgggccctgaagaagggccc-3'
adRNA BoxB dual (targeting domain length 20bp,
mismatch position after 5, 14 bases)
(SEQ ID NO: 149)
3'-CTTCTTTTCAAAATGTTTGACTCGCCACAGACACTCTGAAA-5'
(SEQ ID NO: 157)
5'-ggGCCCTGAAGAAGGGCccCAAACCGCGGTGTCTGTGggGCCCT
GAAGAAGGGCcc-3'

A coordinate or alternate approach to preventing off-target effects is to make use of endogenous ADAR. ADAR2 is highly expressed in tissues such as the brain, lung and spleen while ADAR1 is ubiquitously expressed with general expression levels being higher than ADAR1. Thus, Applicants propose two avenues in order to engineer RNA editing by endogenous ADARs. First, ADAR1 expression can be stimulated by molecules such as interferons, e.g., interferon α. Second, scaffolds may be engineered specifically for recruiting ADAR1 and are carrying out experiments with the v1-v13 scaffolds as well as some chemically modified scaffolds disclosed herein above. Making use of the endogenous ADARs as opposed to overexpression could help limit the off-target effects.

### Recombinant Expression Systems and Vectors

Aspects of the disclosure relate to vectors and recombinant expression systems.

For example, some aspects relate to a vector encoding one or more tRNA having an anticodon sequence that recognizes a codon comprising a point mutation in an RNA sequence encoding a protein, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding tRNA synthetase. In some embodiments, the corresponding synthetase is E. coli Glutaminyl-tRNA synthetase. In some embodiments involving an orthogonal tRNA, the non-canonical amino acid is pyrrolysine. In some embodiments, the vector encodes two tRNA having an anticodon sequence that recognizes the codon comprising the point mutation. In some embodiments, the vector is an AAV vector, optionally an AAV8 vector. In some embodiments, the protein is dystrophin.

Further aspects relate to a recombinant expression system comprising one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits a point mutation in an RNA sequence encoding a protein. In some embodiments, the point mutation results in a nonsense mutation, optionally a premature stop codon, having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, the point mutation results in a splice site or missense mutation having the DNA sequence CAG and the RNA sequence CAG. In some embodiments, the ADAR based RNA editing system converts CAG to CIG. In further embodiments, the one or more vector further encodes a tRNA that targets an amber codon. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA.

In general methods of packaging genetic material such as RNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector (optionally AAV8). The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

The resulting packaged expression systems may then be introduced via an appropriate route of administration, discussed in detail with respect to the method aspects disclosed herein.

### Compositions

Further aspects relate to a composition comprising any one or more of the vectors disclosed herein. In some embodiments, the composition further comprises an effective amount of an interferon to enhance endogenous ADAR1 expression. In still further embodiments, the interferon is interferon α.

Briefly, pharmaceutical compositions of the present disclosure including but not limited to any one of the claimed compositions may comprise a target cell population as described herein, in combination with one or more pharmaceutically or physiologically acceptable carriers, diluents or excipients.

Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

Such compositions may also comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The vectors, recombinant expression systems, and/or compositions are administered to the host using methods known in the art. This administration of the compositions of the disclosure can be done to generate an animal model of the desired disease, disorder, or condition for experimental and screening assays.

Briefly, pharmaceutical compositions of the present disclosure including but not limited to any one of the claimed compositions may comprise one or more vectors or recombinant expression systems as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Pharmaceutical compositions of the present disclosure may be administered in a manner appropriate to the disease, disorder, or condition to be treated or prevented. The quantity and frequency of administration will be determined by such factors as the condition of the patient, and the type and severity of the patient's disease, although appropriate dosages may be determined by clinical trials.

### Methods of Restoring Protein Expression

Aspects of the disclosure relate to methods of restoring protein expression.

For example, some aspects of the disclosure relate to a method for restoring expression of a protein comprising a point mutation in an RNA sequence encoding the protein in a subject in need thereof comprising administering a vector encoding one or more tRNA having an anticodon sequence that recognizes a codon comprising the point mutation to the subject, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding tRNA synthetase. In some embodiments, the corresponding synthetase is E. coli Glutaminyl-tRNA synthetase. In some embodiments involving an orthogonal tRNA, the non-canonical amino acid is pyrrolysine. In further embodiments, the pyrrolysine is introduced in the diet of the subject. In some embodiments, the vector encodes two tRNA having an anticodon sequence that recognizes the codon comprising the point mutation. In some embodiments, the protein is dystrophin.

Other aspects relate to a recombinant expression system comprising one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits a point mutation in an RNA sequence encoding a protein. In some embodiments, the point mutation results in a nonsense mutation, optionally a premature stop codon, having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, optionally those involving nonsense or missense mutations, the RNA targeted in mRNA. In further embodiments, the one or more vector further encodes a tRNA that targets an amber codon. In some embodiments, the protein is dystrophin. In some embodiments, the point mutation results in a splice site or missense mutation having the DNA sequence CAG and the RNA sequence CAG. In some embodiments, the ADAR based RNA editing system converts CAG to CIG. In some embodiments, optionally those involving splice site mutations, the RNA targeted is pre-mRNA. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA.

In either case, the assessment of whether protein expression is “restored” is achieved through any means of protein quantification when compared to a baseline. The baseline may optionally be calculated based on a prior level in the subject or as the normal level in the population, adjusted for the subject's age, ethnicity, and other relevant demographic information. Techniques of quantifying protein expression are well known in the art and may, optionally, utilize a control or a threshold value for comparison to the baseline value. Methods known in the art for such studies include but are not limited to qRTPCR, ELISA, Western blot, protein immunostaining, spectroscopy and/or spectrometry based methods, and other assays typically conducted to determine the amount of protein expression in a sample from the subject. Alternatively, the “restoration” effect may be determined based on a clinical outcome. For example, aberrant dystrophin levels are linked to muscular dystrophy symptoms. Thus, the restoration of expression may be outwardly determined based on clinical signals such as a reduction or reversal of these symptoms. For dystrophin, improvement in muscle strength can be one such indicator. Thus, physicians may carry out strength measurements to determine outcome. Another example is ornithine transcarbamylase (OTC); aberrant OTC levels are a result of a rare X-linked genetic disorder resulting in excessive accumulation of ammonia in the blood (due to nitrogen accumulation). Thus, a relevant clinical outcome would be a decrease in ammonia in a biological sample, such as blood or urine. Similarly, clinical signals associated with and expression of proteins downstream of the protein of interest may be relevant indicators of “restoration” where the protein of interest is involved in a particular pathway.

### Methods of Treatment

Point mutations are implicated in a number of diseases, disorders, and conditions. Non-limiting examples are provided in Table 1 below.

TABLE 1

| | |
|---|---|
| Protein/Disease, Disorder, or Condition | Associated Point Mutation |

G to A point mutations or premature stop codons

| | |
|---|---|
| Dihydropyrimidine dehydrogenase deficiency | NM_000110.3(DPYD): c.1905 + 1G > A |
| Noonan syndrome | NM_005633.3(SOS1): c.2536G > A (p.Glu846Lys) |
| Lynch syndrome | NM_000251.2(MSH2): c.212 − 1G > A |
| Breast-ovarian cancer, familial 1 | NM_007294.3(BRCA1): c.963G > A (p.Trp321Ter) |
| Cystic fibrosis | NM_000492.3(CFTR): c.57G > A (p.Trp19Ter) |
| Anemia, due to G6PD deficiency | NM_000402.4(G6PD): c.292G > A (p.Val98Met) |
| AVPR2 Nephrogenic diabetes insipidus, X-linked | NM_000054.4(AVPR2): c.878G > A (p.Trp293Ter) |
| FANCC Fanconi anemia, complementation group C | NM_000054.4(AVPR2): c.878G > A (p.Trp293Ter) |
| FANCC Fanconi anemia, complementation group C | NM_000136.2(FANCC): c.1517G > A (p.Trp506Ter) |
| IL2RG X-linked severe combined immunodeficiency | NM_000206.2(IL2RG): c.710G > A (p.Trp237Ter) |
| F8 Hereditary factor VIII deficiency disease | NM_000132.3(F8): c.3144G > A (p.Trp1048Ter) |
| LDLR Familial hypercholesterolemia | NM_000527.4(LDLR): c.1449G > A (p.Trp483Ter) |
| CBS Homocystinuria due to CBS deficiency | NM_000071.2(CBS): c.162G > A (p.Trp54Ter) |
| HBB betaThalassemia | NM_000518.4(HBB): c.114G > A (p.Trp38Ter) |
| ALDOB Hereditary fructosuria | NM_000035.3(ALDOB): c.888G > A (p.Trp296Ter) |
| DMD Duchenne muscular dystrophy | NM_004006.2(DMD): c.3747G > A (p.Trp1249Ter) |
| SMAD4 Juvenile polyposis syndrome | NM_005359.5(SMAD4): c.906G > A (p.Trp302Ter) |
| BRCA2 Familial cancer of breast\|Breast-ovarian | NM_000059.3(BRCA2): c.582G > A (p.Trp194Ter) |
| cancer, familial 2 | |
| GRIN2A Epilepsy, focal, with speech disorder and | NM_000833.4(GRIN2A): c.3813G > A (p.Trp1271Ter) |
| with or without mental retardation | |
| SCN9A Indifference to pain, congenital, autosomal | NM_002977.3(SCN9A): c.2691G > A (p.Trp897Ter) |
| recessive | |
| TARDBP Amyotrophic lateral sclerosis type 10 | NM_007375.3(TARDBP): c.943G > A (p.Ala315Thr) |
| CFTR Cystic fibrosis\|Hereditary pancreatitis\|not | NM_000492.3(CFTR): c.3846G > A (p.Trp1282Ter) |
| provided\|ataluren response - Efficacy | |
| UBE3A Angelman syndrome | NM_130838.1(UBE3A): c.2304G > A (p.Trp768Ter) |
| SMPD1 Niemann-Pick disease, type A | NM_000543.4(SMPD1): c.168G > A (p.Trp56Ter) |
| USH2A Usher syndrome, type 2A | NM_206933.2(USH2A): c.9390G > A (p.Trp3130Ter) |
| MEN1 Hereditary cancer-predisposing syndrome | NM_130799.2(MEN1): c.1269G > A (p.Trp423Ter) |
| C8orf37 Retinitis pigmentosa 64 | NM_177965.3(C8orf37): c.555G > A (p.Trp185Ter) |
| MLH1 Lynch syndrome | NM_000249.3(MLH1): c.1998G > A (p.Trp666Ter) |
| TSC2 Tuberous sclerosis 2\|Tuberous sclerosis | NM_000548.4(TSC2): c.2108G > A (p.Trp703Ter) |
| syndrome 46 | |
| NF1 Neurofibromatosis, type 1 | NM_000267.3(NF1): c.7044G > A (p.Trp2348Ter) |
| MSH6 Lynch syndrome | NM_000179.2(MSH6): c.3020G > A (p.Trp1007Ter) |
| SMN1 Spinal muscular atrophy, type II\|Kugelberg- | NM_000344.3(SMN1): c.305G > A (p.Trp102Ter) |
| Welander disease | |
| SH3TC2 Charcot-Marie-Tooth disease, type 4C | NM_024577.3(SH3TC2): c.920G > A (p.Trp307Ter) |
| DNAH5 Primary ciliary dyskinesia | NM_001369.2(DNAH5): c.8465G > A (p.Trp2822Ter) |
| MECP2 Rett syndrome | NM_004992.3(MECP2): c.311G > A (p.Trp104Ter) |
| ADGRV1 Usher syndrome, type 2C | NM_032119.3(ADGRV1): c.7406G > A (p.Trp2469Ter) |
| AHI1 Joubert syndrome 3 | NM_017651.4(AHI1): c.2174G > A (p.Trp725Ter) |
| PRKN Parkinson disease 2 | NM_004562.2(PRKN): c.1358G > A (p.Trp453Ter) |
| COL3A1 Ehlers-Danlos syndrome, type 4 | NM_000090.3(COL3A1): c.3833G > A (p.Trp1278Ter) |
| BRCA1 Familial cancer of breast\|Breast-ovarian | NM_007294.3(BRCA1): c.5511G > A (p.Trp1837Ter) |
| cancer, familial 1 | |
| MYBPC3 Primary familial hypertrophic | NM_000256.3(MYBPC3): c.3293G > A (p.Trp1098Ter) |
| cardiomyopathy | |
| APC Familial adenomatous polyposis 1 | NM_000038.5(APC): c.1262G > A (p.Trp421Ter) |
| BMPR2 Primary pulmonary hypertension | NM_001204.6(BMPR2): c.893G > A (p.W298*) |

T to C point mutations

| | |
|---|---|
| Wilson disease | NM_000053.3(ATP7B): c.3443T > C (p.Ile1148Thr) |
| Leukodystrophy, hypomyelinating, 2 | NM_020435.3(GJC2): c.857T > C (p.Met286Thr) |
| Alport syndrome, X-linked recessive | NM_000495.4(COL4A5): c.438 + 2T > C |
| Leigh disease | NC_012920.1: m.9478T > C |
| Gaucher disease, type 1 | NM_001005741.2(GBA): c.751T > C (p.Tyr251His) |
| Renal dysplasia, retinal pigmentary dystrophy, | NM_014714.3(IFT140): c.4078T > C (p.Cys1360Arg) |
| cerebellar ataxia and skeletal dysplasia | |
| Marfan syndrome | NM_000138.4(FBN1): c.3793T > C (p.Cys1265Arg) |
| Deficiency of UDPglucose-hexose-1-phosphate | NM_000155.3(GALT): c.482T > C (p.Leu161Pro) |
| uridylyltransferase | |
| Familial hypercholesterolemia | NM_000527.4(LDLR): c.694 + 2T > C |
| Episodic pain syndrome, familial, 3 | NM_001287223.1(SCN11A): c.1142T > C (p.Ile381Thr) |
| Navajo neurohepatopathy | NM_002437.4(MPV17): c.186 + 2T > C |
| Congenital muscular dystrophy, LMNA-related | NM_170707.3(LMNA): c.1139T > C (p.Leu380Ser) |
| Hereditary factor VIII deficiency disease | NM_000132.3(F8): c.5372T > C (p.Met1791Thr) |
| Insulin-dependent diabetes mellitus secretory | NM_014009.3(FOXP3): c.970T > C (p.Phe324Leu) |
| diarrhea syndrome | |
| Hereditary factor IX deficiency disease | NM_000133.3(F9): c.1328T > C (p.Ile443Thr) |
| Familial cancer of breast, Breast-ovarian cancer, | NM_000059.3(BRCA2): c.316 + 2T > C |
| familial 2, Hereditary cancerpredisposing syndrome | |
| Cardiac arrhythmia | NM_000238.3(KCNH2): c.1945 + 6T > C |
| Tangier disease | NM_005502.3(ABCA1): c.4429T > C (p.Cys1477Arg) |
| Dilated cardiomyopathy IAA | NM_001103.3(ACTN2): c.683T > C (p.Met228Thr) |
| Mental retardation 3, X-linked | NM_005334.2(HCFC1): c. −970T > C |
| Limb-girdle muscular dystrophy, type 2B | NM_003494.3(DYSF): c.1284 + 2T > C |
| Macular dystrophy, vitelliform, 5 | NM_016247.3(IMPG2): c.370T > C (p.Phe124Leu) |
| Retinitis pigmentosa | NM_000322.4(PRPH2): c.736T > C (p.Trp246Arg) |

Further non-limiting examples include Ornithine Transcarbamylase Deficiency, Nougaret night blindness, Usher syndrome, Atrial Fibrillation, Duchenne Muscular Dystrophy, Wilson disease, hereditary tyrosinemia, and some cancers carrying a A->G mutation in genes such as B-catenin.

Thus, aspects of this disclosure relate to the treatment of certain diseases, disorders, and conditions involving point mutations.

For example, some method aspects relate to a treating a disease, disorder, or condition characterized by the presence of a point mutation in an RNA sequence encoding a protein associated with the disease, disorder, or condition in a subject in need thereof comprising administering a vector encoding one or more tRNA having an anticodon sequence that recognizes a codon comprising the point mutation to the subject, optionally wherein the point mutation results in a premature stop codon. In some embodiments, the point mutation results in a nonsense mutation having the DNA sequence TAA and the RNA sequence UAA. In some embodiments, the tRNA is an endogenous tRNA with a modified anticodon stem recognizing the codon comprising the point mutation. In further embodiments, the tRNA is charged with a serine. In some embodiments, the tRNA is an orthogonal tRNA charged with a non-canonical amino acid. In further embodiments, the vector further comprises a corresponding tRNA synthetase. In some embodiments, the corresponding synthetase is E. coli Glutaminyl-tRNA synthetase. In some embodiments involving an orthogonal tRNA, the non-canonical amino acid is pyrrolysine. In further embodiments, the pyrrolysine is introduced in the diet of the subject. In some embodiments, the vector encodes two tRNA having an anticodon sequence that recognizes the codon comprising the point mutation. In some embodiments, the disease, disorder, or condition is selected from the group consisting of the diseases, disorders, and conditions listed in Table 1, optionally characterized by the presence of a nonsense mutation and/or a premature stop codon. In some embodiments, the protein is dystrophin. In further embodiments, the disease, disorder, or condition is muscular dystrophy. In still further embodiments, the disease disorder or condition is Duchenne muscular dystrophy.

Additional method aspects relate to a method of treating a disease, disorder, or condition by the presence of a point mutation in an RNA sequence encoding a protein associated with the disease, disorder, or condition in a subject in need thereof comprising administering one or more vectors encoding an ADAR based RNA editing system comprising one or more forward guide RNAs for the ADAR (“adRNAs”) and one or more corresponding reverse guide RNAs for the ADAR (“radRNAs”) to the subject, wherein the ADAR based RNA editing system specifically edits the point mutation. In some embodiments, the point mutation results in a nonsense mutation, optionally a premature stop codon, having the DNA sequence TAA and the RNA sequence UAA. In some embodiments, the ADAR based RNA editing system converts UAA to UIA and, optionally, further UIA to UII. In some embodiments, the ADAR based RNA editing system converts UAA to UAI. In some embodiments, optionally those involving nonsense or missense mutations, the RNA targeted in mRNA. In further embodiments, the one or more vector further encodes a tRNA that targets an amber codon. In some embodiments, the protein is dystrophin. In some embodiments, the point mutation results in a splice site or missense mutation having the DNA sequence CAG and the RNA sequence CAG. In some embodiments, the ADAR based RNA editing system converts CAG to CIG. In some embodiments, optionally those involving splice site mutations, the RNA targeted is pre-mRNA. In some embodiments, the ADAR based editing system further comprises ADAR1, ADAR2, the E488Q and E100Q mutants each thereof, a fusion protein comprising the catalytic domain of an ADAR and a domain which associates with an RNA hairpin motif, a fusion protein comprising the catalytic domain of an ADAR and a dead Cas9, or a fusion protein comprising the double stranded binding domain of an ADAR and an APOBEC. In further embodiments, the domain which associates with an RNA hairpin motif is selected from the group of an MS2 bacteriophage coat protein (MCP) and an N22 peptide. In some embodiments, the method further comprises administering an effective amount of an interferon to enhance endogenous ADAR1 expression. In still further embodiments, the interferon is interferon α. In some embodiments, the adRNA comprises one or more RNA hairpin motifs. In some embodiments, the one or more RNA hairpin motifs are selected from the group of an MS2 stem loop and a BoxB loop and/or are stabilized by replacing A-U with G-C. In some embodiments, the adRNA is stabilized through the incorporation of one or more of 2′-O-methyl, 2′-O-methyl 3′phosphorothioate, or 2′-O-methyl 3′thioPACE at either or both termini of the adRNA. In some embodiments, the disease, disorder, or condition is selected from the group consisting of the diseases, disorders, and conditions listed in Table 1. In further embodiments, the protein is dystrophin and the disease, disorder, or condition is muscular dystrophy. In still further embodiments, the disease disorder or condition is Duchenne muscular dystrophy.

An ordinary skilled artisan will appreciate that the doses and route of administration employed in these methods may vary based on the subject and the disease, disorder, or condition to be treated. Based on knowledge in the art such suitable doses and routes may be selected based on the subject's age, ethnicity, and other relevant demographic factors.

### Kits

In one particular aspect, the present disclosure provides kits for performing any of the methods disclosed herein as well as instructions for carrying out the methods of the present disclosure and/or administering the vectors, recombinant expression systems, and compositions disclosed herein.

The kit can also comprise agents necessary for the preservation of those components comprised therein, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components can be packaged in a manner customary for use by those of skill in the art. For example, these suggested kit components may be provided in solution or as a liquid dispersion or the like.

### EXAMPLES

The following examples are non-limiting and illustrative of procedures which can be used in various instances in carrying the disclosure into effect. Additionally, all reference disclosed herein are incorporated by reference in their entirety.

### Example 1—Design of tRNA Constructs

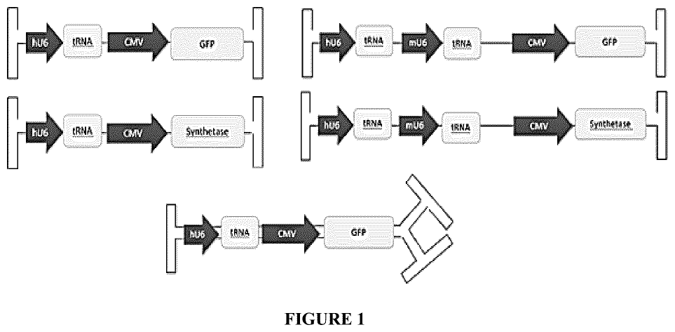

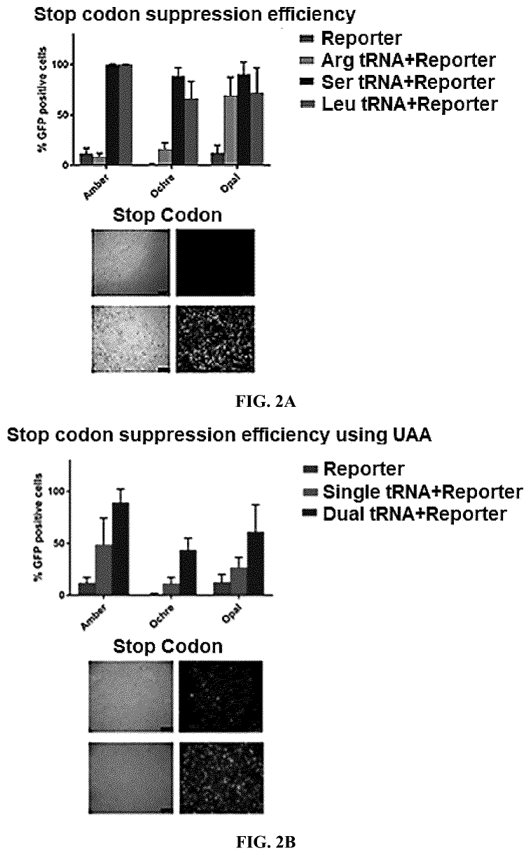

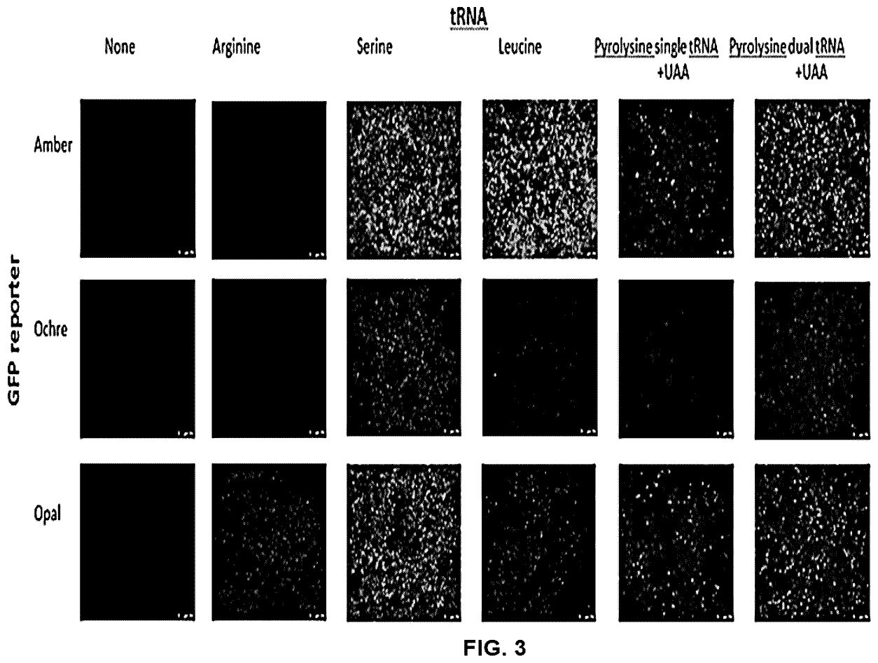

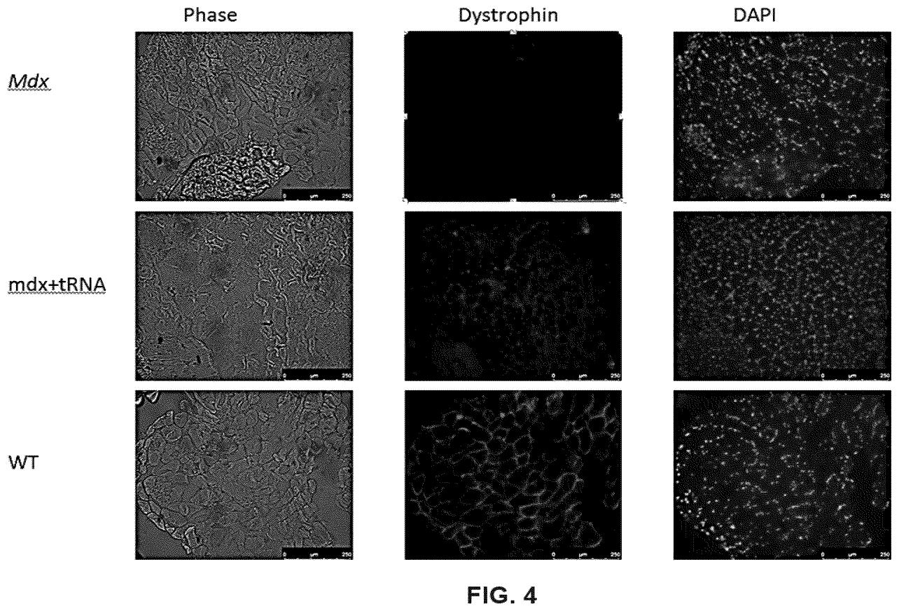

The tRNA constructs were designed along the lines of the schematics in FIG. 1 to recognize the nonsense mutation TAA. Both modified endogenous and orthogonal tRNA were generated. The constructs were validated in vitro using a GFP harboring nonsense mutation. It was determined that two copies of the tRNA should be include in each AAV vector for both modified endogenous and orthogonal tRNAs. MbPyl sythetase was selected for use with the orthogonal tRNA. The AAV vectors were generated comprising the tRNA and GFP (as well as the synthetase, where orthogonal tRNA was used). The sequences used in these constructs are provided in the Sequence Listing above.

### Example 2—Restoration of Full Length Dystrophin in mdx Mice

The anticodon stem of the human serine tRNA is modified such that it recognizes the nonsense codon (TAA). No endogenous tRNA can recognize a stop codon and translation terminates when the ribosome reaches a stop codon. Mdx mice bear a nonsense mutation (TAA) in the gene coding for dystrophin as a result of which they lack full length dystrophin expression. AAVs are used to deliver two copies of the modified tRNAs into the mouse muscle which in turn allows for the stop codon read-through enabling the expression of full length dystrophin.

The calf muscles of mdx mice were injected with 1E12 particles of AAV8 carrying 2 copies of the modified serine tRNA and a GFP gene. These mice were then sacrificed after a month and the calf muscles were harvested. The muscles were sectioned and stained with an antibody against dystrophin. A clear restoration of dystrophin expression was noticed. In addition, the muscle morphology improved too.

Applicants have, thus, demonstrated activity of our vectors in vitro using a GFP gene harboring a stop codon. In addition Applicants have demonstrated restoration of dystrophin expression in mdx mouse muscles. Within a span of one month after injecting the mdx mice with AAVs carrying two copies of the serine tRNA, Applicants observed restoration of dystrophin expression in the calf muscle via Immunohistochemistry. Applicants also noted an improved muscle morphology.

This experiment is repeated with the orthogonal tRNA, introducing the pyrrolysine through the mouse feed, and is again replicated with both tRNAs in a larger population of mice.

### Example 3—Diet Regulable Production of Therapeutic Proteins

Applicants aim at achieving on-demand, in vivo production of therapeutics such as (i) insulin; (ii) neutralizing antibodies for viruses (e.g. HIV, HCV, HPV, influenza) and bacteria (e.g. staph aureus; drug resistant strains) by the skeletal muscle.

The desired transgenes are delivered to the muscle via AAVs (or lentiviruses) along with an orthogonal tRNA/tRNA synthetase pair. These transgenes contain a premature termination codon (stop codon) that will prevent the full length protein from being expressed. For an on demand synthesis of the therapeutics, an appropriate unnatural amino acid is consumed in the diet, which in turn is incorporated by the orthogonal tRNA/tRNA synthetase at the premature termination site, enabling synthesis of the desired therapeutics.

### Example 4—ADAR2 Based RNA Editing

Applicants suspected that ADAR2 (adenosine deaminase that acts on RNA) to correct mutations at the mRNA level. Applicants used Adeno-Associated Viruses to deliver the ADAR2 and a adRNA (forward ADAR2 guide) or radRNA (reverse ADAR2 guide) that direct the enzyme to the mutation in an attempt to restore the expression of dystrophin in the mdx mouse models of DMD, by editing the nonsense mutation. Applicants also applied this technology to the mouse model of the metabolic disorder Ornithine Transcarbamylase (OTC) deficiency.

As compared to nucleases, ADARs make site specific Adenosine to Inosine (A->I) changes in the mRNA with Inosine being read as a Guanosine (G) during translation and are thereby safe from creating permanent off target effects. Also, since they make edits at the mRNA level, the altered proteins are expressed only transiently. The use of nucleases in adult OTC-deficient mice led to large deletions that proved to be lethal to the animals. The use of ADARs might circumvent this problem. This could be a readily translatable solution for several disorders characterized by point mutations. In addition, the origin of the ADAR2 is human, thereby minimizing the immune response generated by the body against it. Applicants also combine the idea of tRNA suppression with ADAR2 based RNA editing. In addition, Applicants designed hairpin loops (3′ overlap) and toe-holds (5′ overlap) that help improve the specificity of the adRNA/radRNA. Applicants also go on to optimize the lengths of the adRNA for efficient A->I editing as well as the ADAR2 recruiting domain of the adRNA/radRNA.

Existing studies have made use of nucleases such as Cas9 to delete the mutated region of the Dystrophin/OTC genes and replaced it with a functional copy, for the treatment of DMD or OTC deficiency caused by a point mutation. For DMD, existing therapies include the use of corticosteroids that delay the symptoms of the disorder. Other strategies include the premature stop codon read-through by making use of drugs such as Ataluren or Gentamycin. Another strategy is that of exon skipping which results in a truncated protein, however able to reduce the severity of the DMD phenotype. Another approach is the delivery of a u-dystrophin gene. Clinical trials for OTC deficiency have been attempted making use of adenoviral vectors to deliver OTC cDNA in patients. Other avenues for treatment include use of sodium phenylbutyrate which helps increase the waste nitrogen excretion.

The use of ADAR2 as an engineered RNA editing enzyme has been demonstrated only in vitro.

Applicants utilized adRNAs and radRNAs comprised of two domains, one complementary to the target sequence and the other an ADAR2 recruiting domain. Applicants utilized AAVs to deliver these adRNAs/radRNAs along with the ADAR2 enzyme. Mdx mice bear a nonsense mutation (TAA) in the gene coding for dystrophin. Applicants packaged two copies of the adRNAs/radRNAs or a combination of adRNA/radRNA+tRNA along with the ADAR2 enzyme into the AAV and deliver it into the Tibialis Anterior (TA) muscle. Applicants utilized three alternative strategies to restore dystrophin expression:
(1) adRNAs/radRNAs that can edit both the adenosines in the ‘TAA’ to inosines;
(2) a sequential approach whereby the first adRNA/radRNA converts TAA->TGA and the next adRNA/radRNA converts it to TGG, restoring expression; and
(3) a combination of adRNAs/radRNAs and a tRNA whereby the adRNA/radRNA converts the TAA into TAG and the tRNA suppression of the amber codon (TAG) restores dystrophin expression.

Applicants also delivered two copies of the adRNA targeting the OTC G->A mutation in spf-ash mice along with the ADAR2 to the liver via retro-orbital injections.

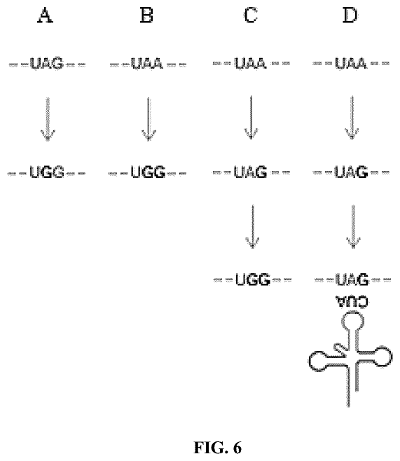

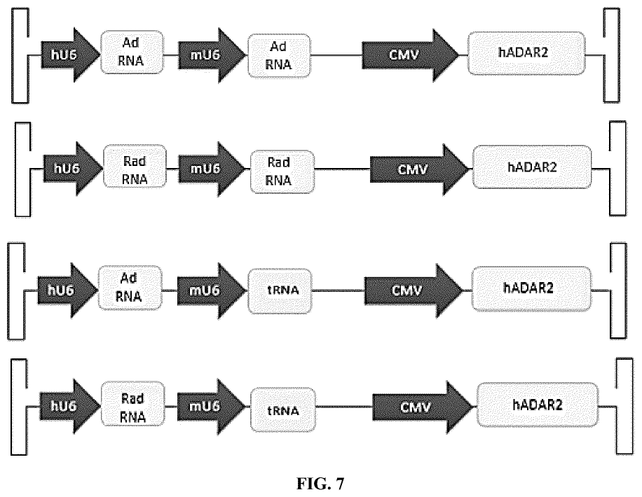

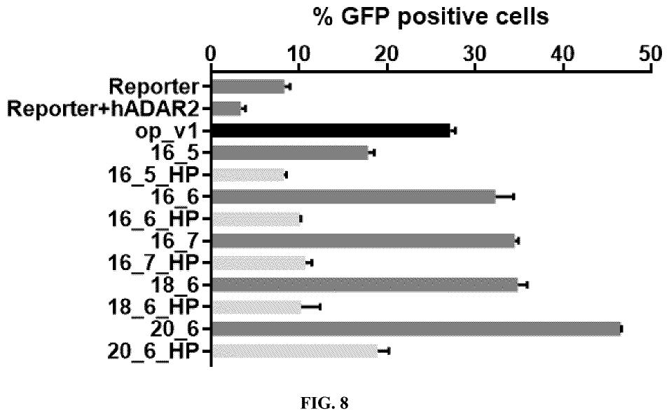

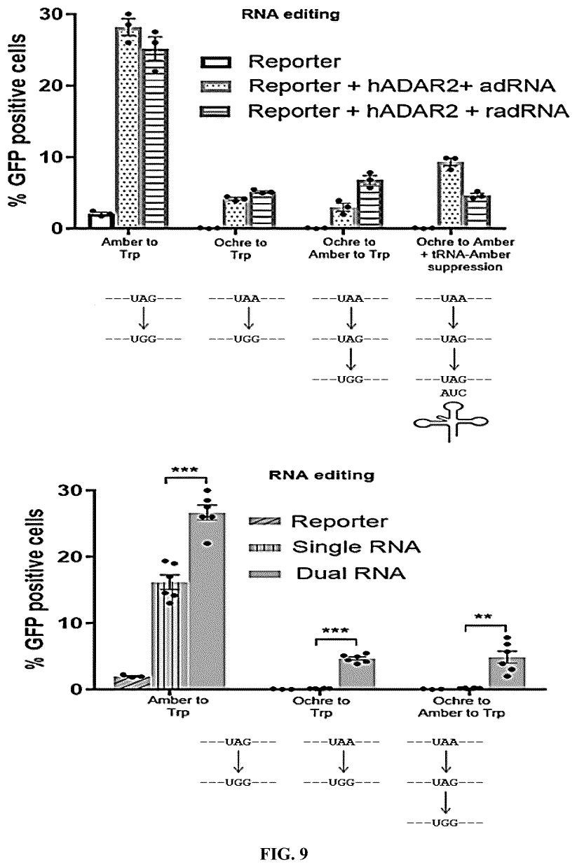

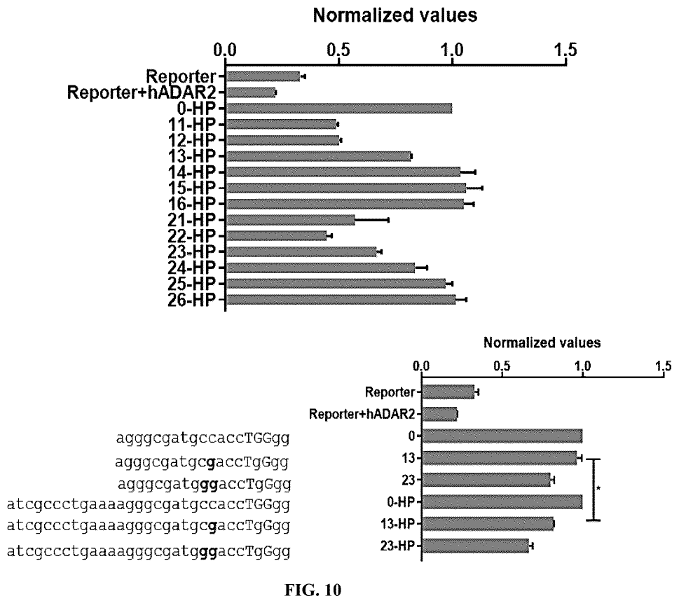

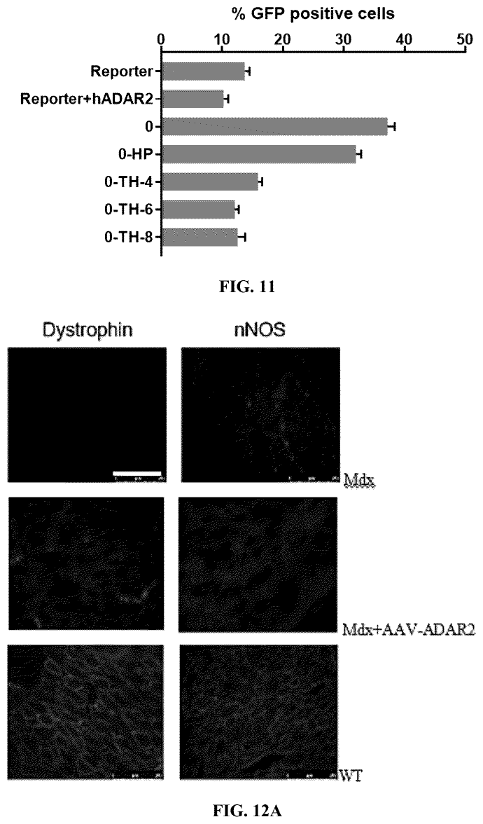

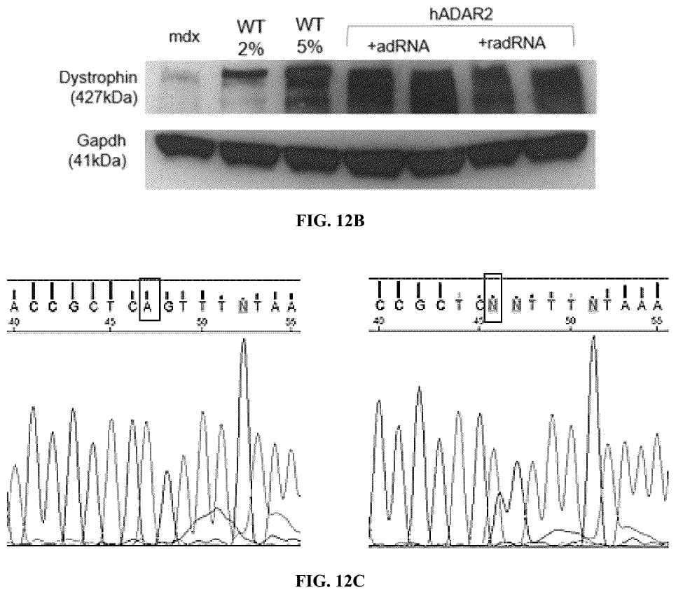

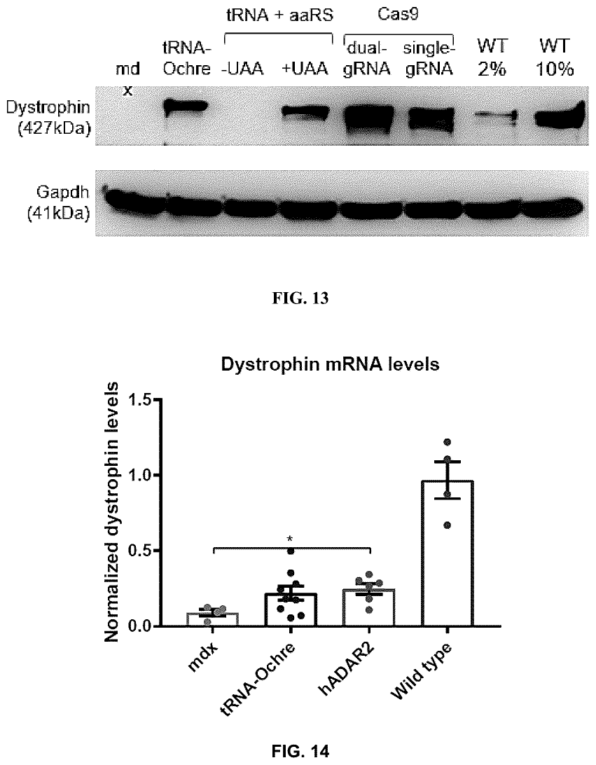

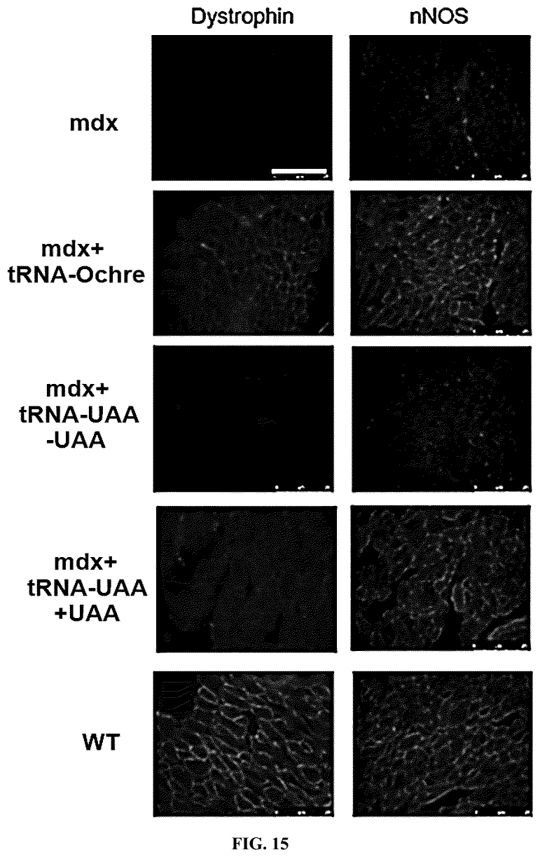

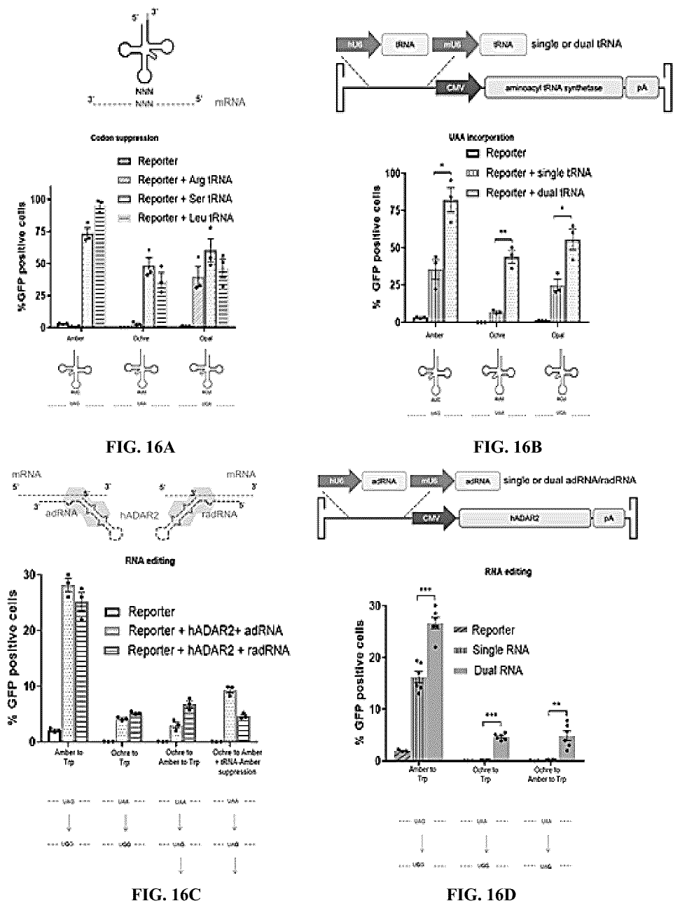

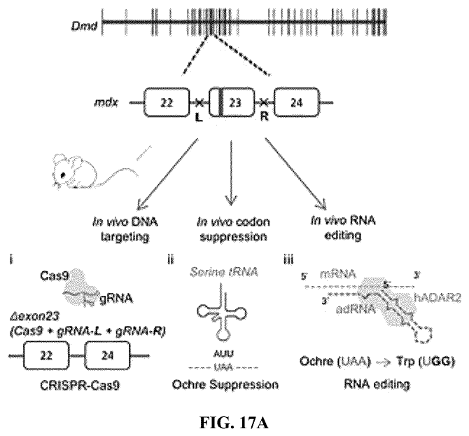

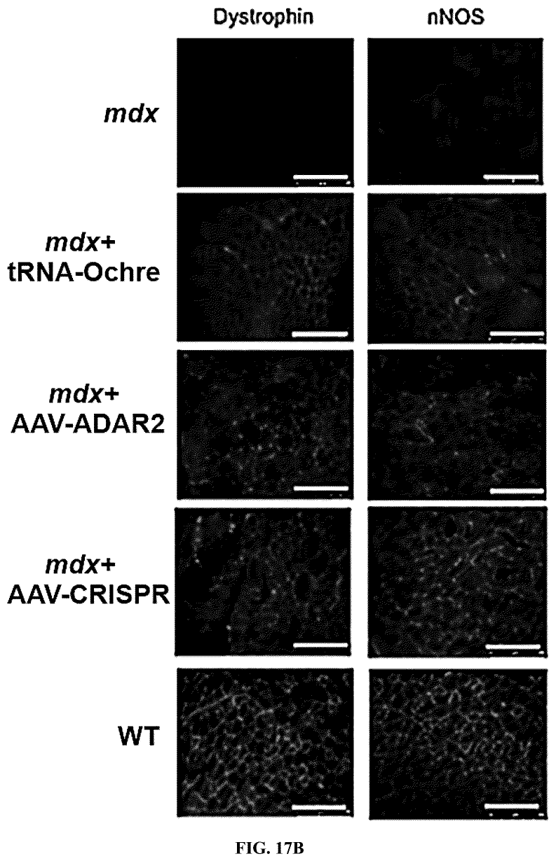

The system works by editing an Adenosine to Inosine which is read as a Guanosine during translation. This can be used to correct point mutations as well as restore expression by editing premature stop codons. In FIG. 6: A. An Amber stop codon can be converted to a tryptophan codon by a single edit. B. Ochre stop codon—both edits made in a single step. C. Ochre stop codon—Sequential editing. D. Ochre stop codon—ADAR2 editing in combination with suppressor tRNA.

The following 10 steps represent the workflow to test these constructs:
1. Design and clone ADAR2 constructs—adRNA and radRNA.
2. In vitro validation of constructs using a GFP harboring a nonsense mutation.
3. Modification of constructs—decision to clone two copies of the adRNA/radRNA. Creation of vectors harboring one copy of a adRNA/radRNA and a copy of a serine suppressor tRNA.
4. Generation of AAV8 vectors carrying ADAR2 and adRNAs/radRNAs or suppressor tRNAs.
6. TA/Gastrocnemius injections of mdx mice—1E12 particles of AAV8 carrying the ADAR2 and with adRNAs/radRNAs or suppressor tRNA were injected.
7. The mice were sacrificed 6 weeks after injections and the TA/gastrocnemimus were harvested. Immunohistochemistry performed to detect dystrophin. Some evidence of restoration of dystrophin.
8. qPCR, Western blots and NGS were carried out.
9. Vectors were optimized to improve efficiency. adRNA lengths varied, location of the edit varied.
10. Steps 4-8 repeated with optimized vectors.

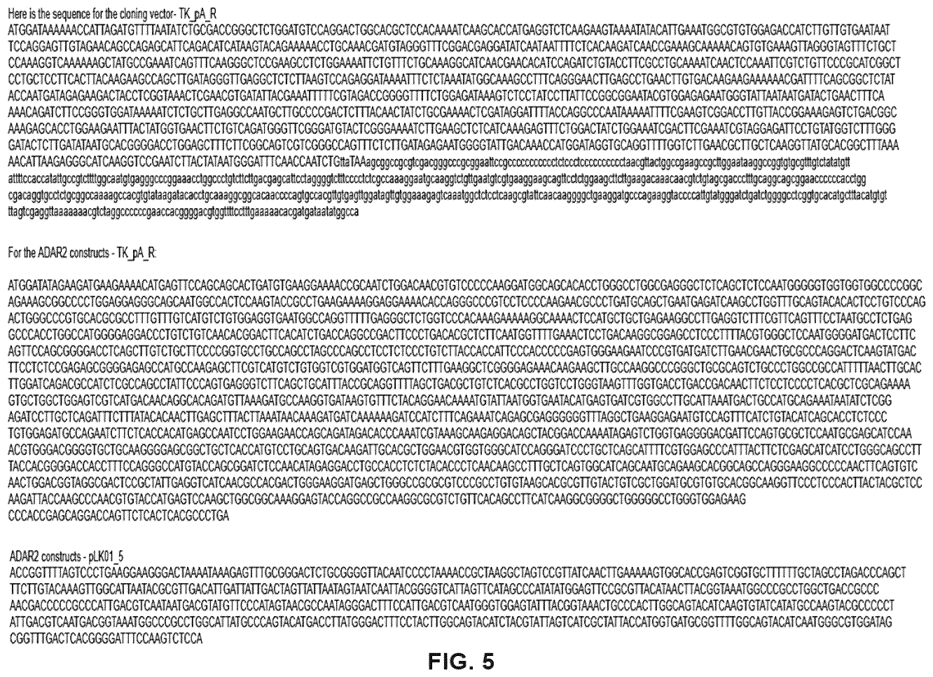

Applicants designed adRNA and radRNAs against a premature stop codon in GFP and demonstrated robust restoration of expression (FIG. 5). For the ochre stop codon (TAA), two A->G edits are needed to restore expression. Applicants constructed a single ad/radRNA targeting both As or a two ad/radRNAs that target a single A in a sequential manner. Applicants also constructed an adRNA/radRNA+suppressor tRNA vector combining RNA editing with tRNA suppression.

In vitro RNA editing showed robust restoration of GFP expression after which AAVs bearing the ADAR2 and adRNA/radRNAs were generated to target the nonsense mutation in dystrophin in mdx mice.

The Tibialis Anterior (TA) or gastrocnemius muscles of mdx mice were injected with 1E12 particles of AAV8 carrying ADAR2 and two copies of the adRNA/radRNA or one copy of the adRNA/radRNA and a suppressor tRNA. These mice were sacrificed after 6 weeks and the appropriate muscles were harvested. The muscles were sectioned and stained with an antibody against dystrophin. Partial restoration of dystrophin expression was noticed.

In general, Applicants noticed a fractional restoration of dystrophin expression via Immunostaining. However, western blots and NGS did not show any evidence of editing/restoration of dystrophin expression.

Potential applications of the system include targeting point mutations for the treatment of disorders such as but not restricted to DMD, OTC deficiency, Wilson's disease and hereditary tysosinemia type 1. It could also be used to create alternate start codons, enabling the co-existence of a protein and its N-terminal truncated form.

### Example 5—ADAR Editing in Mouse Models

Genome engineering methodologies coupled with rapidly advancing synthetic biology toolsets are enabling powerful capabilities to perturb genomes for deciphering function, programming novel function, and repairing aberrant function. In particular, programmable DNA nucleases, such as meganucleases, zinc finger nucleases (ZFNs), transcription activator-like effector nucleases (TALENs), and CRISPR-Cas, have been widely used to engineer genomes across a range of organisms. Their use in gene therapy however poses at least three major challenges: one, the efficiency of homologous recombination versus non-homologous end joining is typically low, particularly in post-mitotic cells that comprise the vast majority of the adult body; two, an active nuclease always poses the threat of introducing permanent off-target mutations, thus presenting formidable challenges in both engineering exquisite nuclease specificity without compromising activity, as well as ensuring tight regulation of the nuclease dose and duration in target cells; and three, prevalent programmable nucleases are of prokaryotic origin or bear domains that are of non-human origin raising a significant risk of immunogenicity in in vivo therapeutic applications. The recent advent of base editing approaches is opening an exciting alternative strategy for gene targeting, but demonstrated approaches rely on CRISPR-Cas systems that are of prokaryotic origin. Thus for genomic mutations that lead to alteration in protein function, such as in disease causing gene mutations, approaches to instead directly target RNA would be highly desirable. Leveraging the aspect that single-stranded RNA as compared to double-stranded DNA, is generally more accessible to oligonucleotide mediated targeting without a need for additional enabling proteins, and building on the advances in tRNA mediated codon suppression and genetic code expansion, as well as adenosine deaminase mediated RNA editing, Applicants have engineered and optimized an integrated platform for RNA targeting, and demonstrate its efficacy in in vitro and in vivo applications.

### Vector Design and Construction

To construct the GFP reporters—GFP-Amber, GFP-Ochre and GFP-Opal, three gene blocks were synthesized with ‘TAG’, ‘TAA’ and ‘TGA’ respectively replacing the Y39 residue of the wild type GFP and were cloned downstream of a CAG promoter. One, two, or four copies of the endogenous suppressor tRNAs were cloned into an AAV vector containing a human U6 and mouse U6 promoter. Pyrrolysyl tRNAs and adRNAs/radRNAs were similarly cloned into an AAV vector containing a human U6 and mouse U6 promoter along with a CMV promoter driving the expression of MbPylRS/MmPylRS/AcKRS or hADAR2 respectively.

### Mammalian Cell Culture and Transfection

All HEK 293T cells were grown in Dulbecco's Modified Eagle Medium supplemented with 10% FBS and 1% Antibiotic-Antimycotic (Thermo Fisher) in an incubator at 37° C. and 5% CO₂ atmosphere. All in vitro transfection experiments were carried out in HEK 293T cells using the commercial transfection reagent Lipofectamine 2000 (Thermo Fisher). All in vitro suppression and editing experiments were carried out in 24 well plates using 500 ng of reporter plasmid and 1000 ng of the suppressor tRNA/aaRS plasmid or the adRNA/ADAR2 plasmid. Cells were transfected at 30% confluence. Cells were harvested 48 and 72 hours post transfection for quantification of suppression and editing respectively. The UAAs NE-Boc-L-Lysine (Chemimpex) and Ne-Acetyl-L-Lysine (Sigma) were added to the media at the desired concentration before transfection.

### Production of AAV Vectors

Virus was prepared using the protocol from the Gene Transfer, Targeting and Therapeutics (GT3) core at the Salk Institute of Biological Studies (La Jolla, CA). AAV8 particles were produced using HEK 293T cells via the triple transfection method and purified via an iodixanol gradient. Confluency at transfection was about 80%. Two hours prior to transfection, DMEM supplemented with 10% FBS was added to the HEK 293T cells. Each virus was produced in 5×15 cm plates, where each plate was transfected with 7.5 ug of pXR-8, 7.5 of ug recombinant transfer vector, 7.5 ug of pHelper vector using PEI (1 ug/uL linear PEI in 1× DPBS pH 4.5, using HCl) at a PEI: DNA mass ratio of 4:1. The mixture was incubated for 10 minutes at RT and then applied dropwise onto the cell media. The virus was harvested after 72 hours and purified using an iodixanol density gradient ultracentrifugation method. The virus was then dialyzed with 1×PBS (pH 7.2) supplemented with 50 mM NaCl and 0.0001% of Pluronic F68 (Thermo Fisher) using 50 kDA filters (Millipore), to a final volume of ˜1 mL and quantified by qPCR using primers specific to the ITR region, against a standard (ATCC VR-1616).

AAV-ITR-F:
(SEQ ID NO: 158)
5′-CGGCCTCAGTGAGCGA-3′
and
AAV-ITR-R:
(SEQ ID NO: 159)
5′-GGAACCCCTAGTGATGGAGTT-3′.

### RNA Isolation and Next Generation Sequencing Library Preparation

RNA from gastrocnemius or TA muscles of mdx mice or livers of spf^{ash} mice was extracted using the RNeasy Plus Universal Mini Kit (Qiagen), according to the manufacturer's protocol. Next generation sequencing libraries were prepared as follows. cDNA was synthesized using the Protoscript II First Strand cDNA synthesis Kit (New England Biolabs). Briefly, 500 ng of input cDNA was amplified by PCR with primers that amplify 150 bp surrounding the sites of interest using KAPA Hifi HotStart PCR Mix (Kapa Biosystems). PCR products were gel purified (Qiagen Gel Extraction kit), and further purified (Qiagen PCR Purification Kit) to eliminate byproducts. Library construction was done with NEBNext Multiplex Oligos for Illumina kit (NEB). 10 ng of input DNA was amplified with indexing primers. Samples were then pooled and loaded on an Illumina Miseq (150 single-end run) at 5 nM concentrations. Data analysis was performed using CRISPResso.

### Animal Experiments

AAV Injections: All animal procedures were performed in accordance with protocols approved by the Institutional Animal Care and Use Committee (IACUC) of the University of California, San Diego. All mice were acquired from Jackson labs. AAVs were injected into the gastrocnemius or TA muscle of mdx mice (6-10 weeks old) using 2.5E+12 vg/muscle. AAVs were injected into spf^{ash} (10-12 weeks old) mice via retro-orbital injections using 3E+12 vg/mouse.

UAA administration: Mice were fed water containing 20 mg/ml NE-Boc-L-Lysine (Chemimpex) for one month. Mice were also administered the 30 mg Ne-Boc-L-Lysine via IP injections, thrice a week.

### Immunofluorescence

Harvested gastrocnemius or TA muscles were placed in molds containing OCT compound (VWR) and flash frozen in liquid nitrogen. 20 μm sections were cut onto pre-treated histological slides. Slides were fixed using 4% Paraformaldehyde. Dystrophin was detected with a rabbit polyclonal antibody against the N-terminal domain of dystrophin (1:100, Abcam 15277) followed by a donkey anti-rabbit Alexa 546 secondary antibody (1:250, Thermo Fisher).

### Statistical Analysis

All statistical analyses were performed using the software Graphpad Prism and p-values were computed by unpaired two-tailed t tests.

### Results

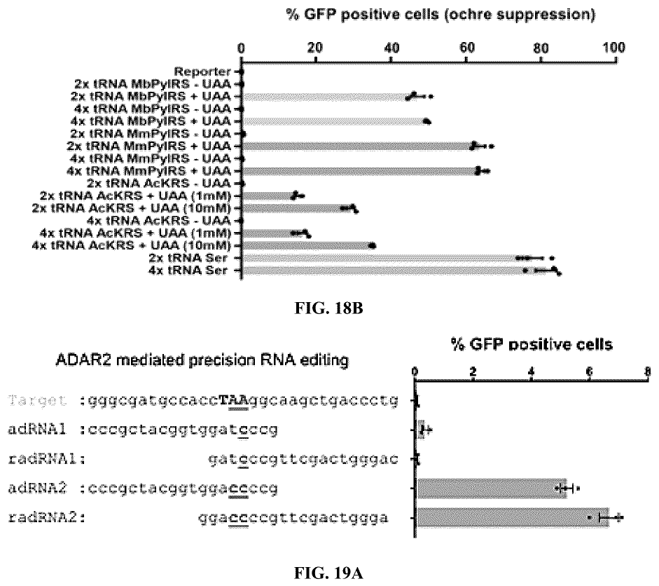

Applicants focused first on establishing the system for targeting nonsense mutations. This was motivated by the fact that nonsense mutations are responsible for 11% of all described gene lesions causing inheritable human disease, and close to 20% of disease-associated single base substitutions that affect the coding regions of genes. Specifically, we explored two independent but complementary approaches to directly target nonsense mutations. First, Applicants focused on engineering robust nonsense codon suppression via suppressor tRNAs. Although the use of suppressor tRNAs for premature stop codon read-through of endogenous non-sense mutations has been attempted in vivo in mice, these prior studies relied only on plasmid delivery and the use of robust and optimized delivery formats was not explored. Additionally, the potential use of un-natural amino acid (UAA) based inducible in vivo suppression of a disease-causing endogenous nonsense mutation has not been explored either. Towards this, Applicants first modified the anticodon stems of serine, arginine and leucine tRNAs to create suppressor tRNAs targeting all three stop codons, amber, opal and ochre, and evaluated these constructs in cells using GFP reporters harboring corresponding nonsense mutations. Among these, the serine suppressor tRNA demonstrated the most consistent and robust results (FIG. 16A, FIG. 18A). To also engineer UAA mediated inducible codon suppression, we next utilized the pyrrolysyl-tRNA/aminoacyl tRNA synthetase (aaRS) pair from Methanosarcina barkeri (MbPylRS)^{32,33} and cloned it into AAV vectors. This enabled programmable incorporation of UAAs at a stop codon. Notably, Applicants found that adding a second copy of the tRNA into the expression vector significantly boosted suppression efficiencies (FIG. 18B). Applicants further systematically evaluated additional aminoacyl tRNA synthetases from Methanosarcina mazei (MmPylRS)³⁴ and an Ne-acetyl-lysyl-tRNA synthetase (AcKRS), and also explored varying the number of tRNAs copies per vector to up to four (FIG. 18B).

As suppressor tRNA based approaches can lead to the read-through of other non-target stop codons, concurrently Applicants also engineered a system for sequence-specific targeted RNA editing via adenosine deaminase enzymes. Specifically, adenosine to inosine (A to I) editing is a common post-transcriptional modification in RNA, catalyzed by adenosine deaminases acting on RNA (ADARs). Inosine is a deaminated form of adenosine and is biochemically recognized as guanine. Recently, multiple studies have demonstrated the engineering of ADAR2 mediated targeting in vitro, and a study also demonstrated correction of the nonsense mutation in CFTR in xenopus oocytes. Building on this, Applicants engineered here a system for sequence-specific targeted RNA editing in vitro and in vivo, utilizing the human ADAR2 enzyme and an associated ADAR2 guide RNA (adRNAs) engineered from its naturally occurring substrate GluR2 pre-mRNA. This ADAR2 guiding RNA comprises an ADAR-recruiting domain and a programmable antisense region that is complementary to a specified target RNA sequence. Applicants first evaluated the RNA editing efficiency of this system in vitro by co-transfecting the constructs with GFP reporters harboring a non-sense amber or ochre mutation at Y39. Specifically, Applicants utilized two editing approaches to engineer the editing of both adenosines in the ochre stop codon: a one-step mechanism where both the adenosines are edited simultaneously or a two-step mechanism wherein editing takes place sequentially. In addition, we also explored the possibility of conversion of an ochre codon to an amber codon followed by amber suppression to restore GFP expression. All three approaches enabled restoration of GFP expression (FIG. 16C, FIG. 19A). Applicants next constructed AAV vectors to deliver the adRNA or a reverse oriented adRNA (radRNA) along with the ADAR2 enzyme. Similar to tRNA mediated codon suppression, addition of a second copy of the adRNA/radRNA significantly improved the targeting efficiency (FIG. 19D). Applicants further systematically evaluated modified ADAR recruiting domains, and a range of RNA targeting antisense designs of varying lengths and the number of nucleotides intervening the target A and the R/G motif of the adRNA²⁶, yielding a panel of efficient adRNA designs (FIG. 19B-C).

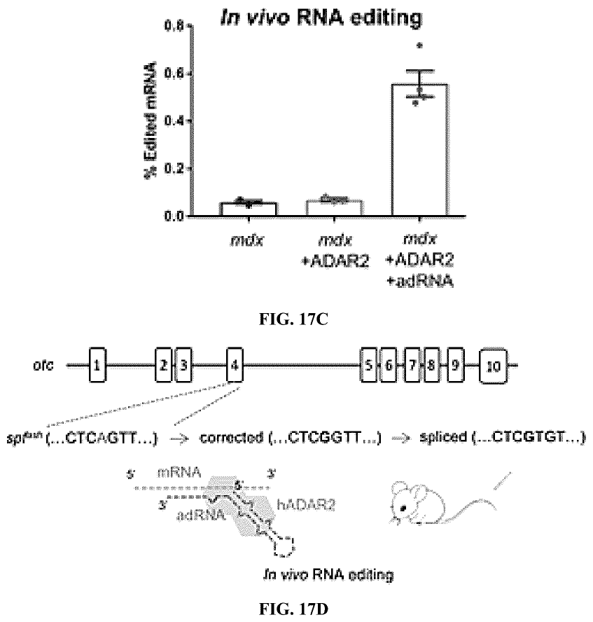

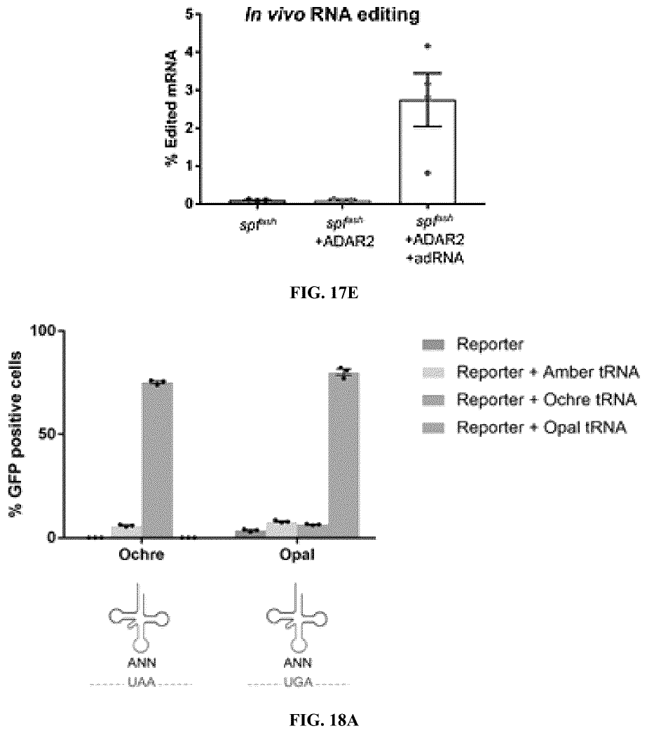

Based on the above in vitro optimizations, Applicants next tested the system for in vivo RNA targeting. Applicants focused first on the mdx mouse model for Duchenne muscular dystrophy (DMD) 35 which bears an ochre stop site in exon 23 of the dystrophin gene. Recent studies utilizing the CRISPR-Cas9 system have shown promising results in the prevention³⁸ and partial functional recovery of DMD by making changes in exon 23 at the DNA level in the mdx mouse. We thus concurrently evaluated three approaches (FIG. 17A): one, suppressor tRNAs derived from modified endogenous tRNAs or pyrrolysyl tRNAs for nonsense codon suppression; two, ADAR2 based correction of the nonsense mutation; and, three, CRISPR-Cas9 based genome targeting to benchmark the RNA targeting approaches.

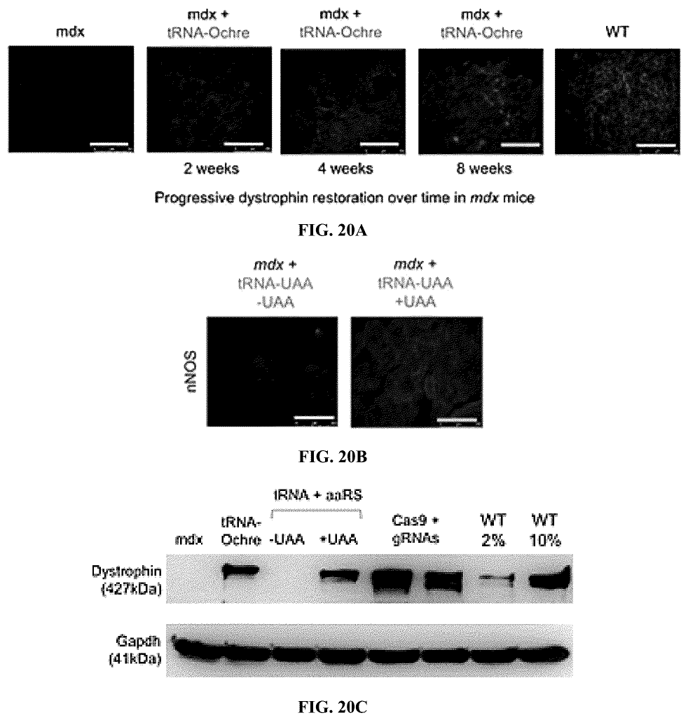

Corresponding, Applicants first designed an AAV carrying two copies of the serine suppressor tRNA targeting the ochre stop codon, and the tibalis anterior (TA) or gastrocnemius of mdx mice were injected with the same. Mice muscles were harvested after 2, 4, and 8 weeks. Progressively improved restoration of dystrophin expression was seen over time, with the mice harvested after 8 weeks showing the greatest degree of restoration (FIG. 17B, FIG. 20A). In addition, neuronal nitric oxide synthase (nNOS) activity was restored at the sarcolemma which is absent in mdx mice due to the absence of the nNOS binding site in the mutant dystrophin protein (FIG. 17B). To further make the system inducible, a vector carrying two copies of the pyrrolysyl-tRNA targeting the ochre stop codon and MbPylRS was also constructed and injected into the TA or gastrocnemius of mdx mice, and the mice were divided into two groups: one that was administered the pyrrolysine UAA and a control group that was not. Expectedly only mice that were provided the UAA showed nNOS localization at the sarcolemma (FIG. 20B), and restoration of dystrophin expression (FIG. 20C).

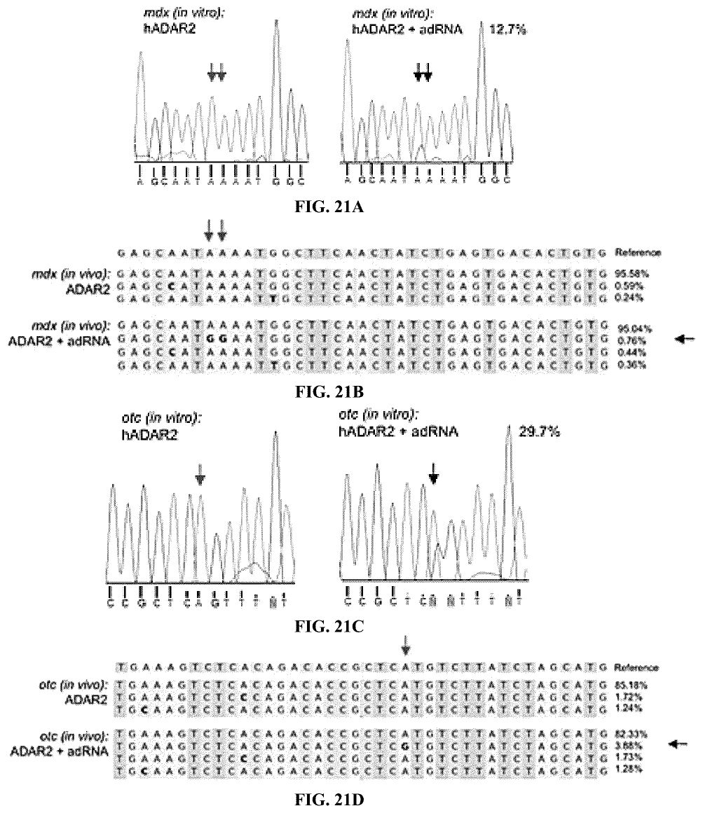

Next, Applicants evaluated the ADAR2 based site-specific RNA editing approach in this mouse model. To test the efficiency of this system in editing both adenosines in the ochre stop codon in mdx DMD mRNA, Applicants first optimized the constructs in vitro with a reporter plasmid bearing a fragment of the mdx DMD mRNA in HEK293T cells. Sanger sequencing and NGS analysis confirmed successful targeting (FIG. 21A). Applicants next packaged the optimized constructs into AAV8, and injected the tibialis anterior (TA) or gastrocnemius of mdx mice. Eight weeks post injection, TA and gastrocnemius muscles were collected from mdx mice, wild type mice, and mice treated with adRNA targeting and non-targeting controls. IHC revealed clear restoration of dystrophin expression (FIG. 17B). In addition, nNOS activity was also restored at the sarcolemma (FIG. 17B). RNA editing rates (TAA->TGG/TAG/TGA) of 0.5-0.7% were observed in treated mice (FIG. 17C, FIG. 21B). Applicants also note that the mdx mice showed no mRNA with a TAA->TGG change while the treated mice showed up to 0.42% TAA->TGG edited mRNA. Applicants note that corresponding DNA editing rates via CRISPR-Cas9 in published in vivo targeting studies were about 2%³⁹. To further benchmark the tRiAD approach, we thus also targeted the mdx mice via CRISPR based genome editing of the nonsense mutation. Applicants injected vectors bearing dual-gRNAs to excise exon 23 codon, and expectedly, this led to restoration of dystrophin expression in a subset of the muscle cells (FIG. 17B).

Finally, we also evaluated the ADAR2 mediated RNA editing approach in an independent mouse model of human disease. Specifically, we focused on the male sparse fur ash (spf^{ash}) mouse model of ornithine transcarbamylase (OTC) deficiency. The spf^{ash} mice have a G->A point mutation in the last nucleotide of the fourth exon of the OTC gene, which leads to OTC mRNA deficiency and production of mutant protein⁴³. Recent studies have demonstrated the use of CRISPR-Cas9 and homologous recombination based strategies for robust correction of this mutation in neonatal mice. However, gene correction via homology-directed repair (HDR) in adult mice was inefficient and led to genomic deletions which proved to be lethal as they compromised liver function in targeted mice. To test the effectiveness of the system in editing the point mutation in spf^{ash} OTC mRNA (FIG. 17D), Applicants first evaluated our constructs in vitro with a plasmid bearing a fragment of the spf^{ash} OTC mRNA in HEK293T cells. Sanger sequencing and next generation sequencing (NGS) analysis confirmed robust RNA editing efficiencies (FIG. 21C). Applicants next packaged the constructs into AAV8, which has high liver tropism⁴⁴, and injected 10-12 week old spf^{ash} mice. Four weeks post injection, Applicants collected liver samples from spf^{ash}, wild-type litter mates, and spf^{ash} mice treated with the ADAR2 targeting and non-targeting vectors and evaluated editing efficiency via NGS. Notably, significant RNA editing rates in the range of 0.8-4.2% were observed in treated mice in the spliced OTC mRNA (FIG. 17E, FIG. 21D), further confirming the utility of this approach for in vivo editing of endogenous RNA in adult mice.

Taken together, Applicants' results establish the use of suppressor tRNAs and ADAR2 as potential strategies for in vivo RNA targeting of point mutations. Specifically, by optimizing delivery, Applicants first demonstrated robust and inducible stop codon read-through via the use of suppressor tRNAs. The delivery of modified endogenous suppressor tRNAs for premature stop read-through has several potential advantages: it lacks the toxicity associated with read-through drugs such as gentamycin and can be used to bring about efficient stop codon read-through in post-mitotic cells. In addition, being of endogenous origin, it is not likely to elicit a strong immune response. Additionally, the inducibility enabled by the UAA based systems, albeit non-native, could provide tight regulation over the expression of genes. Localized injections of the UAA into the target muscle could further help improve the efficiency of the system in future studies. Notably, Applicants did not observe any overt toxicity via this approach in the mdx targeting studies. Applicants however note too that an important caveat to this strategy, analogous to the read-through drugs, is that suppressor tRNA based approaches will lead to the read-through of other non-target stop codons. In this regard, Applicants thus also demonstrated ADAR2 based site-specific correction of point mutations in RNA in two independent mouse models. Applicants note that potential off-targets in RNA are limited as compared to DNA, as the transcriptome is only a small subset of the genome. Secondly, even if off-targets exist, the presence of an A within the target window is required for the enzyme to create an off-target A->G change. Lastly, the off-target effects will be transient. Thus, overall off-target effects due to a RNA editing enzyme such as ADAR2 are likely to be limited, although enzyme processivity, promiscuity, and off-target hybridization of the antisense domain of the adRNA need to be studied thoroughly. ADAR2 being of human origin is also less likely to elicit an immune response, while enabling more site-specific editing of RNA compared to the suppressor tRNA approach.

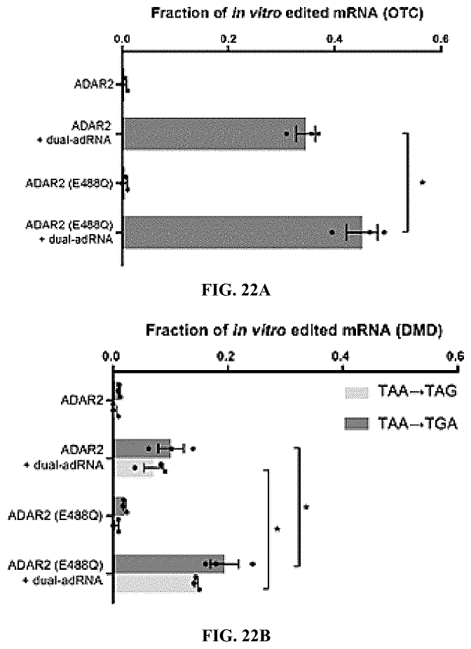

Applicants also note that compared to the tRiAD based RNA targeting approaches above, CRISPR based genome targeting approaches currently show faster kinetics and greater degree of mutant protein restoration. Applicants however anticipate that systematic engineering and directed evolution of the ADAR2 could help improve the editing efficiency and also eliminate the intrinsic biases of the ADAR2 for certain sequences, coupled with insights from studies unearthing novel regulators of ADAR2 providing cues to improve its stability. In this regard, Applicants tested the ADAR2-E488Q mutant and noted that it enabled higher editing efficiency than the wild type ADAR2 for both the DMD and OTC mRNA fragments expressed in vitro (FIG. 22). The demonstration of site-specific A->G mRNA editing in vivo also opens up the door to future site-specific C->T editing via targeted recruitment of cytosine deaminases, thereby potentially expanding the repertoire of RNA editing tools. However, an important consideration while targeting RNA for gene therapy via the use of non-integrating vectors such as AAVs, is the necessity for periodic re-administration of the effector constructs due to the typically limited half-life of edited mRNAs. Secondly, RNA folding, intrinsic half-life, localization, and RNA binding proteins might also impact accessibility of target sites in the RNA. For instance, in this example, the short half-life of mutant dystrophin RNA, and the need to target the transient pre-mRNA in OTC potentially negatively impact overall editing efficiencies. Chemical and structural modifications in tRNAs and adRNAs while taking cues from the specificity studies on sgRNAs⁴⁹, or coupling of shielding proteins, or recently demonstrated programmable RNA binding proteins and RNA-targeting CRISPR-Cas systems, might help improve RNA stability and specificity, and improve the efficiency of the above approaches. With progressive improvements, Applicants thus anticipate this integrated tRiAD toolset will have broad implications in both applied life sciences as well as fundamental research.

### Example 6—ADAR and APOBEC Editing Efficacy

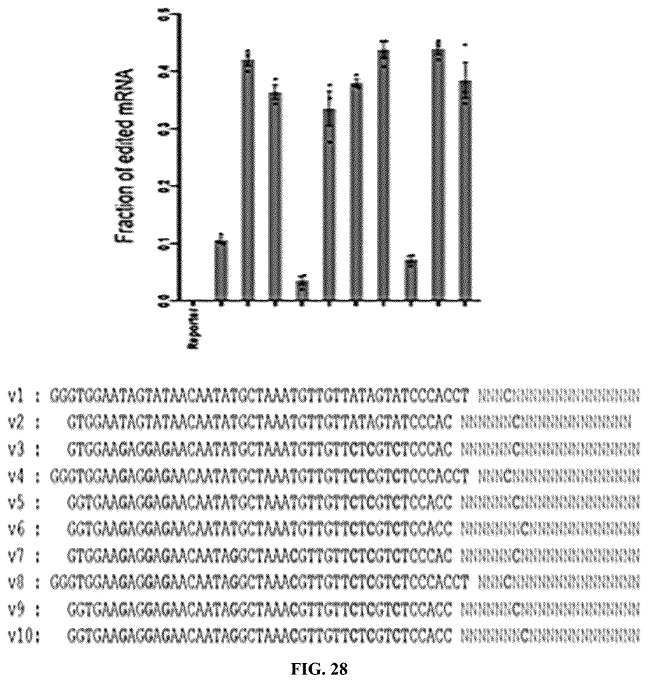

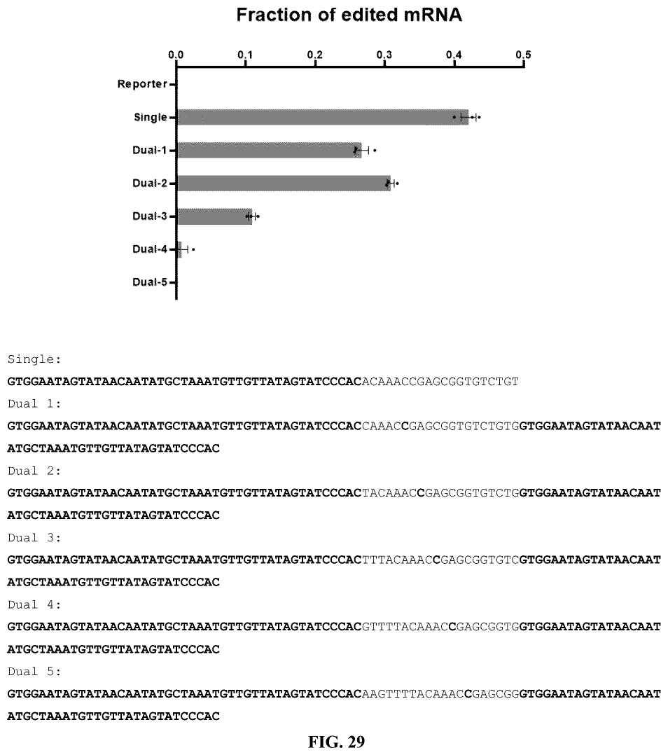

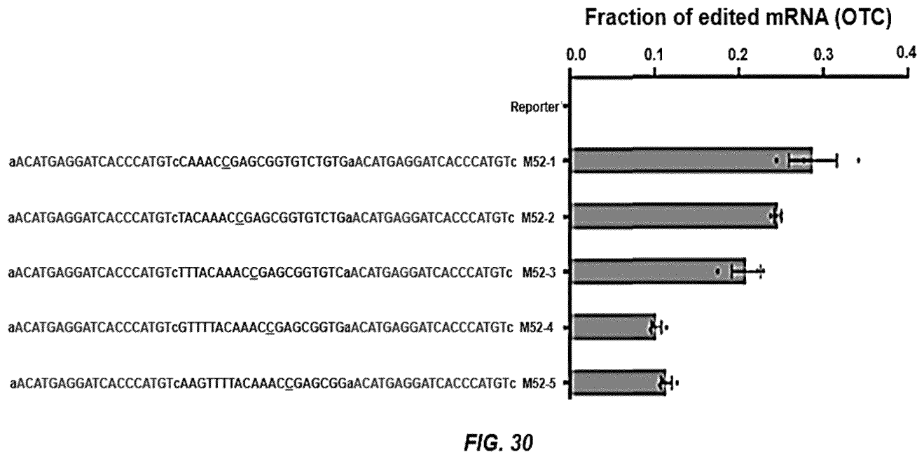

A number of ADAR scaffolds—both dual and single—were tested for efficacy in recruiting ADAR in a cell line where ADAR2 was overexpressed (FIG. 28 and FIG. 29). Further assessments were made for MCP-ADAR fusion scaffolds (FIG. 30). Endogenous mRNA target editing efficiency was assessed using scaffold v2. SEQ ID NOS 160-163 are disclosed below.

| mRNA | Target | #1 | #2 | #3 | Average |
|---|---|---|---|---|---|
| RAB7A | GGGAAATCCAGCTAGCGGCA | 32.0% | 34.1% | 30.2% | 32.1% |
| RAB7A | GGGAAAACTGTCTAGTTCCC | 28.2% | 27.5% | 23.0% | 26.2% |
| CCNB1 | TAATTGACTGGCTAGTACAG | 23.8% | 17.2% | 21.1% | 20.7% |
| CCNB1 | GAGCTTTTTGCTTAGCACTG | 15.1% | 18.4% | 17.4% | 17.0% |

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

Welch, E. M. et al. PTC124 targets genetic disorders caused by nonsense mutations. Nature 447, 87-91 (2007).
Mah, J. Current and emerging treatment strategies for Duchenne muscular dystrophy. Neuropsychiatr. Dis. Treat. Volume 12, 1795-1807 (2016).
Tabebordbar, M. et al. In vivo gene editing in dystrophic mouse muscle and muscle stem cells. Science (80.). 351, 407-411 (2016).
Nelson, C. E. et al. In vivo genome editing improves muscle function in a mouse model of Duchenne muscular dystrophy. Science (80.). 351, (2016).
Cirak, S. et al. Exon skipping and dystrophin restoration in patients with Duchenne muscular dystrophy after systemic phosphorodiamidate morpholino oligomer treatment: an open label, phase 2, dose escalation study. Lancet 378, 595-605 (2011).
Malik, V. et al. Gentamicin induced readthrough of stop codons in Duchenne muscular dystrophy. Ann. Neurol. 67, NANA (2010).
Wagner, K. R. et al. Gentamicin treatment of Duchenne and Becker muscular dystrophy due to nonsense mutations. Ann. Neurol. 49, 706-11 (2001).
Yang, Y. et al. A dual AAV system enables the Cas9-mediated correction of a metabolic liver disease in newborn mice. Nat. Biotechnol. 34, 334-338 (2016).
Wettengel, J. et al. Harnessing human ADAR2 for RNA repair—Recoding a PINK1 mutation rescues mitophagy. Nucleic Acids Res. gkw911 (2016).
Fukuda, M. et al. Construction of a guide-RNA for site-directed RNA mutagenesis utilising intracellular A-to-I RNA editing 1-49.
Hendel, A. et al., Chemically modified guide RNAs enhance CRISPR-Cas genome editing in human primary cells. Nature Biotechnology, 33(9), pp.985-989 (2015).
Jinek, M. et al. A Programmable Dual-RNA-Guided DNA Endonuclease in Adaptive Bacterial Immunity. Science (80-.). 337, 816-821 (2012).
Christian, M. et al. Targeting DNA Double-Strand Breaks with TAL Effector Nucleases. Genetics 186, 757-761 (2010).
Urnov, F. D. et al. Highly efficient endogenous human gene correction using designed zinc-finger nucleases. Nature 435, 646-651 (2005).
Urnov, F. D., Rebar, E. J., Holmes, M. C., Zhang, H. S. & Gregory, P. D. Genome editing with engineered zinc finger nucleases. Nat. Rev. Genet. 11, 636-646 (2010).
Mali, P. et al. RNA-guided human genome engineering via Cas9. Science 339, 823-6 (2013).
Cong, L., Ran, F., Cox, D., Lin, S. & Barretto, R. Multiplex Genome Engineering Using CRISPR/Cas Systems. Science (80). 819, (2013).
Mario, R. et al. Altering the genome by Homologous Recombination. Sci. Virol. Sci. Theor. Appl. Genet. Arch. Tierz. Kexue Tongbao K. Ozato al. Cell Differ. Aquac. Trans. Am. Fish. Soc 244, 1288-1292 (1989).
Takata, M. et al. Homologous recombination and non-homologous end-joining pathways of DNA double-strand break repair have overlapping roles in the maintenance of chromosomal integrity in vertebrate cells. EMBO J. 17, 5497-508 (1998).
Cho, S. W. et al. Analysis of off-target effects of CRISPR/Cas-derived RNA-guided endonucleases and nickases. Genome Res. 24, 132-41 (2014).
Schaefer, K. A. et al. Unexpected mutations after CRISPR—Cas9 editing in vivo
Digenome-seq web tool for profiling CRISPR specificity. Nature 14, 547-548 (2017).
Wang, D. et al. Adenovirus-Mediated Somatic Genome Editing of Pten by CRISPR/Cas9 in Mouse Liver in Spite of Cas9-Specific Immune Responses. Hum. Gene Ther. 26, 432-42 (2015).
Chew, W. L. et al. A multifunctional AAV-CRISPR-Cas9 and its host response. Nat. Methods 13, 868-874 (2016).
Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
Gaudelli, N. M. et al. Programmable base editing of A.T to G.C in genomic DNA without DNA cleavage. (2017). doi:10.1038/nature24644
Kim, K. et al. Highly efficient RNA-guided base editing in mouse embryos. Nat. Biotechnol. 9, 12-15 (2017).
Capone, J. P., Sharp, P. A. & RajBhandary, U. L. Amber, ochre and opal suppressor tRNA genes derived from a human serine tRNA gene. EMBO J. 4, 213-21 (1985).
Geslain, R. & Pan, T. Functional analysis of human tRNA isodecoders. doi:10.1016/j.jmb.2009.12.018
Panchal, R. G., Wang, S., Mcdermott, J. & Link, C. J. Partial Functional Correction of Xeroderma Pigmentosum Group A Cells by Suppressor tRNA. Hum. Gene Ther. 10, 2209-2219 (1999).
Buvoli, M., Buvoli, A. & Leinwand, L. A. Suppression of nonsense mutations in cell culture and mice by multimerized suppressor tRNA genes. Mol. Cell. Biol. 20, 3116-24 (2000).
Wang, L., Brock, A., Herberich, B. & Schultz, P. G. Expanding the Genetic Code of Escherichia coli. Science (80-.). 292, (2001).
Ernst, R. J. et al. Genetic code expansion in the mouse brain. 1-5 (2016). doi:10.1038/nchembio.2160
Han, S. et al. Expanding the genetic code of Mus musculus. Nat. Commun. 8, 14568 (2017).
Melcher, T. et al. A mammalian RNA editing enzyme. Nature 379, 460-464 (1996).
Rueter, S. M., Burns, C. M., Coode, S. A., Mookherjee, P. & Emeson, R. B. Glutamate receptor RNA editing in vitro by enzymatic conversion of adenosine to inosine. Science 267, 1491-4 (1995).
Montiel-Gonzalez, M. F., Vallecillo-Viejo, I., Yudowski, G. A. & Rosenthal, J. J. C. Correction of mutations within the cystic fibrosis transmembrane conductance regulator by site-directed RNA editing. Proc. Natl. Acad. Sci. U.S.A. 110, 18285-90 (2013).
Wettengel, J., Reautschnig, P., Geisler, S., Kahle, P. J. & Stafforst, T. Harnessing human ADAR2 for RNA repair—Recoding a PINK1 mutation rescues mitophagy. Nucleic Acids Res. gkw911 (2016). doi:10.1093/nar/gkw911
Fukuda, M. et al. Construction of a guide-RNA for site-directed RNA mutagenesis utilising intracellular A-to-I RNA editing. Sci. Rep. 7, 41478 (2017).
Mort, M., Ivanov, D., Cooper, D. N. & Chuzhanova, N. A. A meta-analysis of nonsense mutations causing human genetic disease. Hum. Mutat. 29, 1037-1047 (2008).
Bidou, L., Allamand, V., Rousset, J.-P. & Namy, O. Sense from nonsense: therapies for premature stop codon diseases. Trends Mol. Med. 18, 679-688 (2012).
Li, K. et al. OCHRE SUPPRESSOR TRANSFER RNA RESTORED DYSTROPHIN EXPRESSION IN MDX MICE. Life Sci. 61, PL205-PL209 (1997).
Kiselev, A. V. et al. Suppression of nonsense mutations in the Dystrophin gene by a suppressor tRNA gene|Ispol'zovanie gena supressornoi tRNK dlia ispravleniia nonsens-mutatsii v gene distrofina. Mol. Biol. 36, 43-47 (2002).
Gautier, A. et al. Genetically Encoded Photocontrol of Protein Localization in Mammalian Cells. J. Am. Chem. Soc. 132, 4086-4088 (2010).
Chatterjee, A., Xiao, H., Bollong, M., Ai, H. & Schultz, P. G. Ef fi cient viral delivery system for unnatural amino acid mutagenesis in mammalian cells. 110, 11803-11808 (2013).
Greiss, S. & Chin, J. W. Expanding the Genetic Code of an Animal. 2, 14196-14199 (2011).
Robinson-Hamm, J. N. & Gersbach, C. A. Gene therapies that restore dystrophin expression for the treatment of Duchenne muscular dystrophy. Hum. Genet. 135, 1029-1040 (2016).
Bulfield, G., Siller, W. G., Wight, P. A. & Moore, K. J. X chromosome-linked muscular dystrophy (mdx) in the mouse. Proc. Natl. Acad. Sci. U.S.A. 81, 1189-92 (1984).
Sicinski, P. et al. The molecular basis of muscular dystrophy in the mdx mouse: a point mutation. Science 244, 1578-80 (1989).
Long, C. et al. Prevention of muscular dystrophy in mice by CRISPR/Cas9-mediated editing of germline DNA. Science (80-.). 345, 1184-1188 (2014).
Nelson, C. E. et al. In vivo genome editing improves muscle function in a mouse model of Duchenne muscular dystrophy. Science (80-.). 351, (2016).
Tabebordbar, M. et al. In vivo gene editing in dystrophic mouse muscle and muscle stem cells. Science (80-.). 351, 407-411 (2016).
Long, C. et al. Postnatal genome editing partially restores dystrophin expression in a mouse model of muscular dystrophy. Science (80-.). 351, 400-403 (2016).
Bengtsson, N. E. et al. Muscle-specific CRISPR/Cas9 dystrophin gene editing ameliorates pathophysiology in a mouse model for Duchenne muscular dystrophy. Nat. Commun. 8, 14454 (2017).
Hodges, P. E. & Rosenberg, L. E. The spfash mouse: a missense mutation in the ornithine transcarbamylase gene also causes aberrant mRNA splicing. Proc. Natl. Acad. Sci. U.S.A. 86, 4142-4146 (1989).
Yang, Y. et al. A dual AAV system enables the Cas9-mediated correction of a metabolic liver disease in newborn mice. Nat. Biotechnol. 34, 334-338 (2016).
Kuttan, A. & Bass, B. L. Mechanistic insights into editing-site specificity of ADARs. Proc. Natl. Acad. Sci. 109, E3295-E3304 (2012).
Tan, M. H. et al. Dynamic landscape and regulation of RNA editing in mammals. Nature 550, 249-254 (2017).
Varani, G., Cheong, C. & Tinoco, I. Structure of an Unusually Stable RNA Hairpint. Biochemistry 30, 3280-3289 (1991).
Tuerk, C. et al. CUUCGG hairpins: Extraordinarily stable RNA secondary structures associated with various biochemical processes (hairpin stability/sequence analysis/reverse transcriptase). Biochemistry 85, 1364-1368 (1988).
Fu, Y., Sander, J. D., Reyon, D., Cascio, V. M. & Joung, J. K. Improving CRISPR-Cas nuclease specificity using truncated guide RNAs. Nat. Biotechnol. 32, 279-84 (2014).
Adamala, K. P., Martin-Alarcon, D. A. & Boyden, E. S. Programmable RNA-binding protein composed of repeats of a single modular unit. Proc. Natl. Acad. Sci. 113, E2579-E2588 (2016).
East-Seletsky, A. et al. Two distinct RNase activities of CRISPR-C2c2 enable guide-RNA processing and RNA detection. Nature (2016). doi:10.1038/nature19802
Abudayyeh, O. O. et al. C2c2 is a single-component programmable RNA-guided RNA-targeting CRISPR effector. Science 353, aaf5573 1-9 (2016).
O'Connell, M. R. et al. Programmable RNA recognition and cleavage by CRISPR/Cas9. Nature 516, 263-266 (2014).
Abudayyeh, O. O. et al. RNA targeting with CRISPR—Cas13. Nature (2017). doi:10.1038/nature24049
Cox, D. B. T. et al. RNA editing with CRISPR-Cas 13. Science eaaq0180 (2017). doi:10.1126/science.aaq0180
Gootenberg, J. S. et al. Nucleic acid detection with CRISPR-Cas13a/C2c2. Science (80-.). 356, 438-442 (2017).
East-Seletsky, A., O'Connell, M. R., Burstein, D., Knott, G. J. & Doudna, J. A. RNA Targeting by Functionally Orthogonal Type VI-A CRISPR-Cas Enzymes. Mol. Cell 66, 373-383.e3 (2017).
Grieger, J. C., Choi, V. W. & Samulski, R. J. Production and characterization of adeno-associated viral vectors. Nat. Protoc. 1, 1412-1428 (2006). Analyzing CRISPR genome-editing experiments with CRISPResso. Nat. Biotechnol. 34, (2016).

What is claimed is:

1. A pharmaceutical composition comprising:
(a) a guide RNA that:
(i) comprises an antisense region that hybridizes to a target RNA, wherein the antisense region comprises cytosine that, when the guide RNA is hybridized to the target RNA, forms an adenosine-cytosine mismatch with a target adenosine of the target RNA; and
(ii) does not comprise a recruiting domain that is recognized by an adenosine deaminase acting on RNA (ADAR) enzyme; and
(b) pharmaceutically acceptable excipient, diluent, or carrier, wherein an ADAR enzyme present in a cell, when contacted with the guide RNA and the target RNA, results in deamination of the target adenosine in the target RNA.

2. The pharmaceutical composition of claim 1, wherein the target RNA encodes a truncated protein.

3. The pharmaceutical composition of claim 2, wherein administration of the pharmaceutical composition to a subject having a disease or condition caused by the truncated protein results in: (i) treatment of the disease or condition, (ii) restoration of a full length protein from the truncated protein, or (iii) a combination of (i) and (ii).

4. The pharmaceutical composition of claim 1, wherein the target adenosine is present in a premature stop codon, and wherein the deamination of the target adenosine in the premature stop codon converts the premature stop codon into a codon that codes for an amino acid.

5. The pharmaceutical composition of claim 4, wherein the premature stop codon is an opal stop codon or an amber stop codon.

6. The pharmaceutical composition of claim 4, wherein the premature stop codon is an ochre stop codon, and wherein two consecutive target adenosines are deaminated, resulting in conversion of the ochre stop codon into a codon that codes for an amino acid.

7. The pharmaceutical composition of claim 1, wherein the ADAR enzyme that is present in the cell is endogenous to the cell.

8. The pharmaceutical composition of claim 1, wherein the guide RNA comprises a chemical modification.

9. The pharmaceutical composition of claim 8, wherein the chemical modification is a 2′-O-methyl modification, a 2′-O-methyl 3′phosphorothioate modification, or a 2′-O-methyl 3′thioPACE modification.

10. A method of treating a disease or condition in a subject in need thereof, the method comprising administering to the subject in need thereof a guide RNA that:
(a) comprises an antisense region that hybridizes to a target RNA implicated in the disease or condition, wherein the antisense region comprises cytosine that, when the guide RNA is hybridized to the target RNA, forms an adenosine-cytosine mismatch with a target adenosine of the target RNA; and
(b) does not comprise a recruiting domain that is recognized by an adenosine deaminase acting on RNA (ADAR) enzyme;
wherein the administering results in hybridization of the antisense region to the target RNA and recruitment of an ADAR enzyme present in a cell of the subject in need thereof to perform a deamination of the target adenosine of the target RNA, and wherein the deamination of the target adenosine of the target RNA results in treating the disease or condition in the subject in need thereof.

11. The method of claim 10, wherein the administering is oral, intravenous, topical, enteral, or parenteral.

12. The method of claim 10, wherein the subject is human.

13. The method of claim 10, wherein the target RNA encodes a truncated protein.

14. The method of claim 13, wherein administering results in restoration of a full length protein from the truncated protein.

15. The method of claim 10, wherein the target adenosine is present in a premature stop codon, and wherein the deamination of the target adenosine in the premature stop codon converts the premature stop codon into a codon that codes for an amino acid.

16. The method of claim 10, wherein the ADAR enzyme is endogenous to the subject in need thereof.

17. The method of claim 10, wherein the guide RNA comprises a chemical modification.

18. The method of claim 17, wherein the chemical modification is a 2′-O-methyl modification, a 2′-O-methyl 3′phosphorothioate modification, or a 2′-O-methyl 3′thioPACE modification.
